(19) United States
(12) Patent Application Publication
(10) Pub. No.: US 2024/0075050 A1
(43) Pub. Date: Mar. 7, 2024

(54) Methods and Compositions for Treating Leucine Rich Repeat Kinase 2 (LRRK2)-Associated Disorder or Condition

(71) Applicant: University of Connecticut, Farmington, CT (US)

(72) Inventors: Yulan Xiong, West Hartford, CT (US); Jianzhong Yu, West Hartford, CT (US); Qinfang Liu, Manchester, CT (US)

(73) Assignee: University of Connecticut, Farmington, CT (US)

(21) Appl. No.: 18/226,071

(22) Filed: Jul. 25, 2023

Related U.S. Application Data
(60) Provisional application No. 63/392,676, filed on Jul. 27, 2022.

(51) Int. Cl. A61K 31/7056; A61K 45/06; A61P 25/28; A61P 29/00

(52) U.S. Cl. CPC A61K 31/7056; A61K 45/06; A61P 25/28; A61P 29/00

(57) ABSTRACT

The present invention relates to methods and compositions for treating an LRRK2-associate disorder or condition, e.g., Parkinson's Disease, methods for reducing or preventing neuronal cell death, and methods for reducing neurodegeneration and/or neuroinflammation, in a subject in need thereof, by an agent that decreases the expression of LRRK2, e.g., an agent that induces LRRK2 mRNA decay.

### RELATED APPLICATION

This application claims the benefit of priority to U.S. Provisional Application No. 63/392,676, filed on Jul. 27, 2022, the entire contents of which are incorporated hereby by reference.

### STATEMENT OF GOVERNMENT SUPPORT

This invention was made with government support under grant number NS112506 from the National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Nov. 16, 2023, is named 98121_00367_SL.xml and is 30,172 bytes in size.

### BACKGROUND

Parkinson's Disease (PD) is one of the most common neurodegenerative disorders in the world. It affects over one million Americans and more than 60,000 patients are newly diagnosed annually. PD is generally classified by somatic symptoms including tremors, rigidity, bradykinesis, and postural problems. In the early stages of the disease, there may be only slight disturbances of posture, locomotion, facial expressions, or speech. Symptoms may initially manifest as asymmetric, however, as the disease progresses, the symptoms become bilateral and progressively debilitating. PD patients also commonly experience dementia, ataxia, dysphasia, and mood disorders, and the quality and life expectancy of patients with PD is substantially reduced. The neuropathological hallmarks are characterized by a progressive loss of dopaminergic neurons in the substantia nigra pars compacta and the presence of proteinaceous inclusions immunoreactive for α-synuclein termed as Lewy bodies and dystrophic Lewy neurites in surviving neurons. Traditionally, PD has been considered a sporadic neurodegenerative disorder. However, increasing evidence of family aggregation indicates that there may be a link between PD and gene.

To date, many genes have been studied and isolated to be responsible for PD based on family based linkage analysis. Among all the causative genes, mutations in LRRK2 and α-synuclein have been proved to associate with autosomal dominant PD, while mutations in parkin, PINK1, DJ-1, and ATP13A2 are linked to autosomal recessive PD.

Leucine rich repeat kinase 2 (LRRK2) is a large multifunctional protein. Mutations in LRRK2 gene are the most frequent genetic causes of both sporadic and familial Parkinson's disease (PD). Disease causing mutations are found throughout different domains of LRRK2. Importantly, LRRK2-mediated disease is clinically and pathologically indistinguishable from sporadic PD. Given its strong genetic link to PD, LRRK2 represents a clear and compelling target for therapeutic development for PD. However, the mechanisms that regulate LRRK2 function and the pathologic actions responsible for the LRRK2-linked disease remain unclear. Studies have focused largely on the enzymatic activities of LRRK2. Decreased GTPase activity and increased kinase activity of LRRK2 are believed to be the major culprit of cell toxicity. However, pathogenic mutations that do not change LRRK2 enzymatic functions have been reported, suggesting altered enzymatic activities may not be the sole mechanism responsible for the LRRK2-linked PD.

Although tremendous effort has been made to find an effective treatment or cure for PD, most PD patients have experienced little relief from current treatment regimes, which include medications, surgeries, rehabilitation therapy and implants. Many of the benefits provided from standard treatments are relatively insignificant and are often accompanied by appreciable toxicity. The most common current therapy for PD is oral administration of L-DOPA, also known as levodopa and 1-3,4-dihydroxyphenylalanine. However, there are certain limitations associated with its use. It requires large daily dosage; the therapeutic benefits are achieved only after a delayed onset of 1-2 months; and it has a number of side effects both central and peripheral and may cause adverse side effects in patients with cardiovascular or pulmonary disease, asthma, or renal, hepatic, or endocrine disease.

### SUMMARY OF THE INVENTION

The present invention is based, at least in part, on the discovery and generation of novel regulators of LRRK2 expression. In particular, the present inventors surprisingly identified 5-aminoimidazole-4-carboxamide ribonucleotide formyltransferase/IMP cyclohydrolase (ATIC), the last enzyme in the de novo purine biosynthesis pathway, as a novel regulator that regulates LRRK2 expression levels and toxicity. In addition, the present inventors also discovered that both AICAR/ZMP (5-aminoimidazole-4-carboxamide ribonucleotide), the enzymatic substrate of ATIC, and AICAr (5-aminoimidazole-4-carboxamide riboside), the precursor of AICAR/ZMP, can regulate LRRK2 expression levels through an mRNA decay pathway. Furthermore, the inventors discovered that neurodegeneration and inflammation, for example, associated with Parkinson's Disease, can be treated by modulating AICAr and AICAR/ZMP mediated-LRRK2 expression. Thus, the present invention provides a novel regulatory mechanism of regulating LRRK2 through LRRK2 mRNA decay and establishes the modulation of LRRK2 mRNA decay as a potential novel therapeutic strategy for LRRK2 associated disorders and conditions, such as Parkinson's Disease, which is distinct from targeting enzymatic functions of LRRK2.

Accordingly, the present invention provides, in one aspect, a method of treating a Leucine rich repeat kinase 2 (LRRK2)-associated disorder or condition in a subject in need thereof, comprising administering to the subject an effective amount of an agent that decreases the expression of LRRK2, wherein the agent induces LRRK2 mRNA decay, thereby treating the LRRK2-associated disorder or condition in the subject.

In another aspect, the present invention provides a method of reducing or preventing neuronal cell death in a subject in need thereof, comprising administering to the subject an effective amount of an agent that decreases the expression of LRRK2, wherein the agent induces LRRK2 mRNA decay, thereby reducing or preventing neuronal cell death in the subject.

In some embodiments, the neuronal death is LRRK2-mediated neuronal cell death. In one aspect, the present invention provides a method of reducing neurodegeneration and/or neuroinflammation in a subject in need thereof, comprising administering to the subject an effective amount of an agent that decreases the expression of LRRK2, wherein the agent induces LRRK2 mRNA decay, thereby reducing neurodegeneration and/or neuroinflammation in the subject.

In some embodiments, the subject has an LRRK2-associated disorder or condition.

In some embodiments, the LRRK2-associated disorder or condition is a neurodegenerative disease. In some embodiments, the neurodegenerative disease is Parkinson's disease, or Alzheimer's disease.

In some embodiments, the LRRK2-associated disorder or condition is an inflammatory disease. In some embodiments, the inflammatory disease is selected from the group consisting of Crohn's disease, inflammatory bowel disease, ulcerative colitis, leprosy, amyotrophic lateral sclerosis, rheumatoid arthritis, and ankylosing spondylitis.

In some embodiments, the LRRK2-associated disorder or condition is a cancer. In some embodiments, the cancer is selected from the group consisting of kidney cancer, breast cancer, prostate cancer, ovarian cancer, cervical cancer, skin cancer, pancreatic cancer, colorectal cancer, renal cancer, liver cancer, brain cancer, lung cancer, lymphoma, leukemia, multiple myeloma, and any combination thereof.

In some embodiments, the subject is a human subject.

In some embodiments, the agent is an inhibitor of 5-aminoimidazole-4-carboxamide ribonucleotide formyltransferase (ATIC). In some embodiments, the ATIC inhibitor is selected from the group consisting of a small molecule, an antagonist antibody of ATIC, or antigen-binding fragment thereof, an antisense agent targeting ATIC, a double stranded RNA agent targeting ATIC, an RNA-guided nuclease targeting ATIC, an ATIC fusion protein; and an ATIC inhibitory peptide.

In some embodiments, the agent is 5-aminoimidazole-4-carboxamide ribonucleotide (AICAR/ZMP).

In some embodiments, the agent is 5-aminoimidazole-4-carboxamide riboside (AICAr).

In some embodiments, the agent increases the expression and/or activity of AU-rich element RNA binding protein 1 (AUF1). In some embodiments, the agent is selected from the group consisting of a small molecule activator of AUF1, an agonist antibody of AUF1, or antigen-binding fragment thereof, an AUF1 protein, a nucleic acid encoding the AUF1 protein, or a protein and a nucleic acid that activates the transcription and/or translation of AUF1.

In some embodiments, the agent increases the expression and/or activity of mRNA decapping enzyme 1 (DCP1) and/or mRNA decapping enzyme 2 (DCP2). In some embodiments, the agent is selected from the group consisting of a small molecule activator of DCP1 and/or DCP2, an agonist antibody of DCP1 and/or DCP2, or antigen-binding fragment thereof, a DCP1 and/or DCP2 protein, a nucleic acid encoding the DCP1 and/or DCP2 protein, or a protein and a nucleic acid that activates the transcription and/or translation of DCP1 and/or DCP2.

In some embodiments, the agent does not modulate the enzymatic activity of LRRK2.

In some embodiments, the methods further comprise administering to the subject an additional therapeutic agent. In some embodiments, the additional therapeutic agent comprises levodopa, carbidopa, a dopamine agonist, a monoamine oxidase B (MAO B) inhibitor, a catechol O-methyltransferase (COMT) inhibitor, an anticholinergic, or an adenosine receptor antagonist.

In one aspect, the present invention provides a method of reducing LRRK2 expression in a cell, comprising contacting the cell with an agent that induces LRRK2 mRNA decay, thereby reducing LRRK2 expression in the cell.

In some embodiments, the cell is a neuron, a microglia, or a fibroblast.

In some embodiments, the contacting occurs in vitro. In some embodiments, the cell is within a subject.

In some embodiments, the subject is a human subject.

In some embodiments, the subject has an LRRK2-associated disorder or condition.

In some embodiments, the LRRK2-associated disorder or condition is a neurodegenerative disease. In some embodiments, the neurodegenerative disease is Parkinson's disease, or Alzheimer's disease.

In some embodiments, the LRRK2-associated disorder or condition is an inflammatory disease. In some embodiments, the inflammatory disease is selected from the group consisting of Crohn's disease, inflammatory bowel disease, ulcerative colitis, leprosy, amyotrophic lateral sclerosis, rheumatoid arthritis, and ankylosing spondylitis.

In some embodiments, the LRRK2-associated disorder or condition is a cancer. In some embodiments, the cancer is selected from the group consisting of kidney cancer, breast cancer, prostate cancer, ovarian cancer, cervical cancer, skin cancer, pancreatic cancer, colorectal cancer, renal cancer, liver cancer, brain cancer, lung cancer, lymphoma, leukemia, multiple myeloma, and any combination thereof.

In some embodiments, the subject is a human subject.

In some embodiments, the agent is an inhibitor of 5-aminoimidazole-4-carboxamide ribonucleotide formyltransferase (ATIC). In some embodiments, the ATIC inhibitor is selected from the group consisting of a small molecule, an antagonist antibody of ATIC, or antigen-binding fragment thereof, an antisense agent targeting ATIC, a double stranded RNA agent targeting ATIC, an RNA-guided nuclease targeting ATIC, an ATIC fusion protein; and an ATIC inhibitory peptide.

In some embodiments, the agent is 5-aminoimidazole-4-carboxamide ribonucleotide (AICAR/ZMP).

In some embodiments, the agent is 5-aminoimidazole-4-carboxamide riboside (AICAr).

In some embodiments, the agent increases the expression and/or activity of AU-rich element RNA binding protein 1 (AUF1). In some embodiments, the agent is selected from the group consisting of a small molecule activator of AUF1, an agonist antibody of AUF1, or antigen-binding fragment thereof, an AUF1 protein, a nucleic acid encoding the AUF1 protein, or a protein and a nucleic acid that activates the transcription and/or translation of AUF1.

In some embodiments, the agent increases the expression and/or activity of mRNA decapping enzyme 1 (DCP1) and/or mRNA decapping enzyme 2 (DCP2). In some embodiments, the agent is selected from the group consisting of a small molecule activator of DCP1 and/or DCP2, an agonist antibody of DCP1 and/or DCP2, or antigen-binding fragment thereof, a DCP1 and/or DCP2 protein, a nucleic acid encoding the DCP1 and/or DCP2 protein, or a protein and a nucleic acid that activates the transcription and/or translation of DCP1 and/or DCP2.

In some embodiments, the agent does not modulate the enzymatic activity of LRRK2.

In one aspect, the present invention provides a method for identifying a compound useful for treating an LRRK2-associated disorder or condition in a subject, comprising providing a test compound; determining the effect of the test compound on the expression and/or activity of LRRK2, AITC, AUF1, DCP1 and/or DPC2; and selecting a compound which decreases the expression and/or activity of LRRK2 and AITC, or a compound which increases the expression and/or activity of AUF1, DCP1 and/or DCP2, thereby identifying a compound useful for treating a cancer in the subject.

In one aspect, the present invention provides a method of identifying a compound useful for reducing or preventing neuronal cell death in a subject in need thereof, comprising providing a test compound; determining the effect of the test compound on the expression and/or activity of LRRK2, AITC, AUF1, DCP1 and/or DPC2; and selecting a compound which decreases the expression and/or activity of LRRK2 and AITC, or a compound which increases the expression and/or activity of AUF1, DCP1 and/or DCP2, thereby identifying a compound useful for reducing or preventing neuronal cell death in the subject.

In one aspect, the present invention provides a method of identifying a compound useful for reducing neurodegeneration and/or neuroinflammation in a subject in need thereof, comprising providing a test compound; determining the effect of the test compound on the expression and/or activity of LRRK2, AITC, AUF1, DCP1 and/or DPC2; and selecting a compound which decreases the expression and/or activity of LRRK2 and AITC, or a compound which increases the expression and/or activity of AUF1, DCP1 and/or DCP2, thereby identifying a compound useful for reducing neurodegeneration and/or neuroinflammation in the subject.

The present invention is illustrated by the following drawings and detailed description, which do not limit the scope of the invention described in the claims.

### BRIEF DESCRIPTION OF THE DRAWINGS

FIGS. 1A-1I depicts that ATIC regulates LRRK2-induced toxicity and expression levels. FIG. 1A depicts a cell viability assay by cell number in response to expression of LRRK2-AN fragment of wildtype (WT) LRRK2 (pYES2-LRRK2-ΔN, 570-2527aa (1708 bp-7581 bp)) or empty vector in WT and ATIC homolog deletion mutants (ade16Δ, ade17Δ, ade16Δ-ade17Δ) BY4741 yeast strains. Cells were spotted onto media containing glucose (LRRK2 Off, repressed, left panel) or galactose (LRRK2 On, induced, right panel) and incubated at 30° C. for 2 to 3 days. Shown are five-fold serial dilutions of yeast cells from left to right as indicated by graded open box. FIG. 1B depicts representative fluorescent images showing that mouse primary cortical neurons were transduced with shRNA-control or shATIC #6 at DIV 3 and then transfected with MYC-LRRK2-WT and DsRed at a plasmid ratio of 10:1 at DIV 5. Neuronal viability was analyzed at 72-hour post-transfection with non-viable neurons exhibiting no obvious neurite process (arrow). Scale bars, 100 μm. FIG. 1C depicts the quantification of neuronal viability. Bars indicate the viability (n>100) for each transfection condition expressed as a percent of control neurons (DsRed with pcDNA3.1 empty vector). Data represent the mean±SEM from three independent experiments with n>100 cells quantified in each experiment. **p<0.01, ***p<0.001 by one-way ANOVA followed by a Tukey's post hoc test. FIGS. 1D and 1E depict the endogenous LRRK2 levels in ATIC overexpression cells. SH-SY5Y cells were transfected with V5-ATIC or empty vector. After 48 hours, LRRK2 expression levels were analyzed by Western blot. Images were quantified by ImageJ software. Data are mean±SEM, n=3 independent experiments. ***p<0.001 by Student's t tests. FIGS. 1F and 1G depict the endogenous LRRK2 levels in ATIC KO cells. ATIC was knocked out in SH-SY5Y cells using CRISPR/Cas9 editing system. LRRK2 expression levels were analyzed in WT, or ATIC KO SH-SY5Y cells by Western blot. Images were quantified by ImageJ software. Data are the means±SEM, n=3 independent experiments. *p<0.05, **p<0.01 by one-way ANOVA followed by a Tukey's post hoc test. FIGS. 1H and 1I depict the endogenous LRRK2 levels in ATIC KO cells upon LV-flag-ATIC expressing. SH-SY5Y WT or ATIC KO cells were infected with lentiviruses carrying Flag-ATIC or empty vector. After 48 hours, LRRK2 expression levels were analyzed by Western blot. Images were quantified by ImageJ software. Data are the means±SEM, n=3 independent experiments. ***p<0.001 by one-way ANOVA followed by a Tukey's post hoc test.

FIGS. 2A-2T depicts that AICAr, the precursor of ATIC substrate, regulates LRRK2 expression levels and neuronal toxicity. FIGS. 2A and 2B depict the overexpressed LRRK2 levels in HEK 293T cells upon AICAr treatment. HEK293 cells were transfected with MYC-LRRK2 and treated with or without 1 mM AICAr for 48 hours. LRRK2 levels were analyzed by Western blot. FIGS. 2C and 2D depict the LRRK2 expression levels in SH-SY5Y cells upon AICAr treatment. Human neuroblastoma SH-SY5Y cells were treated with or without 1 mM AICAr for 48 hours. LRRK2 expression levels were analyzed by Western blot. FIGS. 2E and 2F depict the overexpressed (3-glucocerebrosidase (GCase) levels upon AICAr treatment. HEK 293T cells were transfected with V5-GCase and treated with or without 1 mM AICAr for 48 hours. GCase levels were analyzed by Western blot. FIGS. 2G-2N depict the LRRK2 expression levels in primary neurons (FIG. 2G and FIG. 2H), microglia (FIG. 2I and FIG. 2J), astrocytes (FIG. 2K and FIG. 2L) and human fibroblasts (FIG. 2M and FIG. 2N) upon AICAr treatment. Mouse primary cortical neurons, microglia, astrocytes or human fibroblasts from a health control were treated with or without 1 mM AICAr for 48 hours. LRRK2 expression levels were analyzed by Western blot. FIGS. 2O and 2P depict the overexpressed LRRK2 WT and pathogenic mutant levels upon AICAr treatment. HEK 293T cells were transfected with MYC-LRRK2 WT, R1441C (RC), R1441G (RG), Y1699C (YC), G2019S (GS) or I2020T (IT) and treated with or without 1 mM AICAr for 48 hours. LRRK2 expression levels were analyzed by Western blot. FIGS. 2Q and 2R depict the LRRK2 expression levels in mouse striatum upon AICAr treatment. AICAr (72 μg/day/animal) or saline (used as a control) was injected into mouse striatum via osmotic pumps. One day after injection, striatal tissues near the injection sites were collected and LRRK2 expression levels were analyzed by Western blot. Images were quantified by ImageJ software. Data are mean±SEM from total six mice (three male mice and three female mice). ****p<0.0001 by Student's t tests. FIG. 2S depicts representative fluorescent images showing that mouse primary cortical neurons were co-transfected with MYC-LRRK2 WT or G2019S (GS), and eGFP at a plasmid ratio of 10:1 at DIV 5, and treated with or without 1 mM AICAr. Neuronal viability was analyzed at 48-hour post-transfection with non-viable neurons exhibiting no obvious neurite process (arrow). Scale bars, 100 μm. FIG. 2T depicts the quantification of neuronal viability. Bars indicate the viability (n>100) for each transfection condition expressed as a percent of control neurons (eGFP with pcDNA3.1 empty vector). Data represent the mean±SEM from three independent experiments with n>100 cells quantified in each experiment. *p<0.05, ***p<0.001, ****p<0.0001 by one-way ANOVA followed by a Tukey's post hoc test.

FIGS. 3A-3J depict that AICAr regulates LRRK2 at mRNA level and through a specific region. FIGS. 3A and 3B depict the LRRK2 protein levels upon treatment of AICAr together with a proteasome inhibitor MG132 or an autophagy inhibitor chloroquine (CQ). SH-SY5Y cells were treated with or without 1 mM AICAr, 0.35 μM MG132, and 5 μM CQ as showed in (FIG. 3A) for 48 hours. LRRK2 expression levels were analyzed by Western blot. Images were quantified by ImageJ software. Data are mean±SEM, n=3 independent experiments. ***p<0.001, ****p<0.0001 by one-way ANOVA followed by a Tukey's post hoc test. FIG. 3C depicts the LRRK2 and GBA mRNA levels upon AICAr treatment. SH-SY5Y cells were treated with or without 2 mM AICAr for 24 hours. Total RNA was extracted. LRRK2 and GBA mRNA levels were analyzed by real-time PCR. Data are mean±SEM, n=4 independent experiments. **p<0.01 by Student's t tests. ns: not statistically significant. FIG. 3D depicts the LRRK2 mRNA level upon AICAr treatment in the presence of actinomycin D. SH-SY5Y cells were treated with or without 2 mM AICAr for 24 hours and in the presence of 10 μg/ml actinomycin D. Total RNA was extracted. LRRK2 mRNA levels were analyzed by real-time PCR. Data are mean±SEM, n=4 independent experiments. **p<0.01, ****p<0.0001 by two-way ANOVA followed by Sidak's multiple comparison test. FIGS. 3E and 3F depict the LRRK2 expression levels from native LRRK2 cDNA and synthesized LRRK2 cDNA with synonymous codons upon AICAr treatment. HEK 293T cells were transfected with Flag-LRRK2 with native cDNA or with synthesized cDNA, and treated with or without 1 mM AICAr for 48 hours. LRRK2 expression levels were analyzed by Western blot. Images were quantified by ImageJ software. Data are mean±SEM, n=3 independent experiments. ***p<0.001 by Student's t tests. FIGS. 3G and 3H depict the LRRK2 deletion mutant expression levels upon AICAr treatment. HEK 293T cells were transfected with a series of LRRK2 deletion mutants from C-terminals (G) or N-terminals (H) as illustrated in the diagram and treated with or without 1 mM AICAr for 48 hours. LRRK2 deletion mutant expression levels were analyzed by Western blot. FIGS. 3I and 3J depict that the specific region (2221-3927 bp) of the synthesized Flag-LRRK2 cDNA was replaced with the native sequence and the response of this chimeric LRRK2 cDNA expression to AICAr treatment was analyzed by Western blot. Images were quantified by ImageJ software. Data are mean±SEM, n=3 independent experiments. ****p<0.0001 by Student's t tests.

FIGS. 4A-4H depict AUF1 as a specific LRRK2 RNA binding protein mediates AICAr-regulated LRRK2 levels. FIG. 4A depicts that an RNA-protein pull-down assay of AICAr response element of LRRK2 followed by MS analysis identified AUF1 (HnRNPD) as a specific LRRK2 RNA binding protein. The RNA-protein pull-down assay was performed using a magnetic RNA-protein pull-down kit from Thermo Scientific by incubating an RNA probe from AICAr response element of LRRK2 (2221-3927 bp) or a control region of LRRK2 with lysates from SH-SY5Y cells. The resulting samples were subjected to SDS-PAGE and Coomassie blue staining. The specific band labeled in red box was cut and sent for MS analysis. FIGS. 4B and 4C depict the LRRK2 protein levels in AUF1 knockout (KO) cells upon AICAr treatment. SH-SY5Y WT and AUF1 KO cells were treated with or without 1 mM AICAr for 48 hours. LRRK2 expression levels were analyzed by Western blot. Images were quantified by ImageJ software. FIG. 4D depicts the LRRK2 mRNA levels in AUF1 KO cells upon AICAr treatment. SH-SY5Y WT and KO cells were treated with or without AICAr for 24 hours. Total RNA was extracted. LRRK2 mRNA levels were analyzed by real-time PCR. FIGS. 4E and 4F depict the LRRK2 protein levels in AUF1 KO cells with AUF1 expressing back upon AICAr treatment. SH-SY5Y WT and KO cells were transfected AUF1-GFP or GFP and selected with 4 μg/ml blasticidin. The resulting stable cell lines were treated with or without 1 mM AICAr for 48 hours. LRRK2 expression levels were analyzed by Western blot. FIGS. 4G and 4H depict the overexpressed LRRK2 levels in AUF1 KO cells with AUF1 expressing back upon AICAr treatment. SH-SY5Y WT and AUF1 KO cells were transfected AUF1-GFP or GFP and selected with 4 μg/ml blasticidin. The resulting cell lines were transfected with a construct with native LRRK2 cDNA or synthesized LRRK2 cDNA with synonymous codons and treated with or without 1 mM AICAr for 48 hours. LRRK2 expression levels were analyzed by Western blot. Images were quantified by ImageJ software. Data information: Data are mean±SEM, n=3 independent experiments. *p<0.05, ***p<0.001, ****p<0.0001 by one-way ANOVA followed by a Tukey's post hoc test. ns: not statistically significant.

FIGS. 5A-5D depict that AUF1 interacts with LRRK2 mRNA. FIGS. 5A and 5B depict that AUF1 binds to LRRK2 mRNA upon AICAr treatment. SH-SY5Y WT and AUF1 KO cells were treated with or without 1 mM AICAr for 24 hours. RNA and protein complexes were immunoprecipitated using AUF1 antibody. The LRRK2 mRNA levels were analyzed by semiquantitative reverse transcription (RT)-PCR. 18S ribosomal RNA (rRNA) was as a loading control. Images were quantified by ImageJ software. Data are mean±SEM, n=3 independent experiments. *p<0.05 by Student's t tests. FIG. 5C depicts a schematic representation of four AUF1 isoforms. The AUF1/HNRPD gene has 10 exons. AUF1 isoforms are generated by alternative mRNA splicing. The start codon in exon 1 and the stop codon in exon 8 are highlighted. RRM1, RRM2, and Q-rich domain common to all isoforms are highlighted. RRM: RNA recognition motifs; Q-rich: glutamine-rich. FIG. 5D depicts the direct interaction of LRRK2 mRNA and four AUF1 isoforms. Biotin-labelled 62-nt LRRK2 RNA probes from native LRRK2 cDNA or synthesized LRRK2 cDNA were incubated with purified AUF1 isoforms in an RNA electrophoretic mobility shift assay (EMSA). Free probes and AUF1 bound probes were detected using a chemiluminescent RNA EMSA kit.

FIGS. 6A-6E depict that the decapping enzyme complex DCP1/2 is involved in LRRK2 mRNA decay. FIGS. 6A and 6B depict the DCP1A and DCP2 expression levels upon AICAr treatment. SH-SY5Y cells were treated with or without 1 mM AICAr for 48 hours. DCP1A and DCP2 expression levels were analyzed by Western blot. Images were quantified by ImageJ software. Data are mean±SEM, n=3 independent experiments. **p<0.01 by Student's t tests. FIGS. 6C and 6D depict the LRRK2 expression levels upon DCP1A or DCP2 overexpression. SH-SY5Y cells were transduced with lentiviruses carrying Flag-DCP1A or Flag-DCP2 for 48 hours. LRRK2 expression levels were analyzed by Western blot. Images were quantified by ImageJ software. Data are mean±SEM, n=3 independent experiments. *p<0.05 by Student's t tests. FIG. 6E depict LRRK2 mRNA decapping by DCP1A and DCP2. Recombinant DCP1A and DCP2 proteins were purified from HEK 293T cells and incubated with [α-32P]cap-labeled LRRK2 RNA. LRRK2 mRNA decapping was analyzed by one-dimensional thin layer chromatography. The decapping activity is demonstrated by the increased m7GDP levels.

FIGS. 7A-7G depict that AICAr effect on LRRK2 requires AICAr to be converted to AICAR/ZMP but is independent of AMPK. FIGS. 7A and 7B depict the LRRK2 protein levels upon the treatment of AICAr, AICAr plus an adenosine transporter (AT) inhibitor ABT702 or an adenosine kinase (ADK) inhibitors NBT-1. SH-SY5Y cells were treated with or without 1 mM AICAr, AICAr plus 1 μM ABT-702 or 50 μM NBT-1 for 48 hours. FIGS. 7C and 7D depict the LRRK2 protein levels upon the treatment of AICAr or plus AMPK direct activator A769662. SH-SY5Y cells were treated with or without 1 mM AICAr, 10, 20, or 50 μM A769662 for 48 hours. FIGS. 7E and 7F depict the LRRK2 protein levels upon the treatment of 1 mM AICAr in WT and AMPK KO SH-SY5Y cells for 48 hours. LRRK2 expression levels were analyzed by Western blot. Images were quantified by ImageJ software. FIG. 7G depicts a summary model that AICAr must be internalized into cells and converted to AICAR/ZMP to exert its effects on LRRK2, which is independent of AMPK. Data information: Data are mean±SEM, n=3 independent experiments. **p<0.01, ****p<0.0001 by one-way ANOVA followed by a Tukey's post hoc test. ns: not statistically significant.

FIGS. 8A-8F depicts that AICAr rescues LRRK2-induced DA neurodegeneration in vivo in Drosophila. FIG. 8A depicts a diagram of major DA neuronal clusters (PPM1/2, PPM3, PPL1) in the posterior areas of the adult fly brain. Scale bars, 100 sm. FIG. 8B depicts representative confocal images (GFP) of dopamine neurons in each cluster from 9-week-old WT and LRRK2-G2019S transgenic flies with or without 1 mM AICAr treatment after posteclosion. Scale bars, 25 sm. FIGS. 8C-8E depict the quantification of dopamine neurons per DA cluster in 9-week-old WT and LRRK2-G2019S transgenic flies with or without 1 mM AICAr treatment after posteclosion. FIG. 8F depicts the total numbers of dopamine neurons in four major DA clusters of 9-week-old WT and LRRK2-G2019S transgenic flies with or without AICAr treatment after posteclosion. Data are mean±SEM, n=12 flies per group. *p<0.05, **p<0.01, ***p<0.001, ****p<0.0001 by one-way ANOVA followed by a Tukey's post hoc test.

FIGS. 9A-9F depict that AICAr rescues LPS-induced DA neurodegeneration and inflammation in mice, which acts through LRRK2. FIG. 9A depicts representative images of DAneuron staining. WT and LRRK-KO mice were injected with 1 ul LPS (5 ug/animal) or phosphate buffered saline (PBS) into the substantia nigra pars compacta (SNpc) and administrated with AICAr (2.4 μg/day/animal) or saline to same stereotaxic coordinates using osmotic pumps. After 21 days of injection, DA neurons were stained by anti-tyrosine hydroxylase (TH) antibody. Scale bars, 500 sm. FIG. 9B depicts quantification of TH positive neurons in substantia nigra pars compacta (SNpc) using an unbiased stereological method with stereo investigator software from 7-8 animals per group. FIG. 9C depicts quantification of Niss1-positive neurons in SNpc using an unbiased stereological method with stereo investigator software from 7-8 animals per group. FIG. 9D depicts the CD68 levels in SNpc tissues near the needle sites analyzed by immunostaining. Scale bars, 100 μm. FIG. 9E depicts the CD68 levels in WT and LRRK-KO mice injected with 1 μl LPS (5 μg/animal) or PBS into striatum followed by administration of AICAr (2.4 μg/day/animal) or saline to same stereotaxic coordinates using osmotic pumps. After 21 days of injection, LRRK2 and CD68 expression in striatal tissues near the needle sites was analyzed by Western blot. FIG. 9F depicts quantification of images from (FIG. 9E) using ImageJ software, n=3 animals per group. Data information: Data are mean±SEM. ***p<0.001, ****p<0.0001 by one-way ANOVA followed by a Tukey's post hoc test. ns: not statistically significant.

FIG. 10 depicts a model of an ATIC-AICAR-AUF1 axis in regulation of LRRK2 mRNA decay. Knockout of ATIC or AICAr administration increases the intracellular AICAR/ZMP level, which in turn facilitates AUF1 binding to LRRK2 mRNA, and the decapping enzyme complex DCP1/2 is promoted to decap LRRK2 mRNA, leading to LRRK2 mRNA decay, and finally reduces the LRRK2 protein level. AICAR-mediated LRRK2 protein reduction significantly rescues LRRK2-mediated neuronal toxicity in vitro in primary neuronal culture and in vivo in LRRK2 Drosophila genetic model and LPS mouse PD models of neurodegeneration and neuroinflammation.

FIGS. 11A-11C depict knockdown or knockout of ATIC. FIG. 11A depicts that shATIC #6 significantly suppresses ATIC expression. ATIC expression upon shATIC treatment. NIH 3T3 cells were transfected with shATIC #1/2/3/4/5/6 or shControl and selected with 2 ug/ml puromycin. The ATIC protein levels were analyzed by Western blot. FIG. 11B depicts quantification of images from (FIG. 11A) using ImageJ software. Data are mean±SEM, n=3 independent experiments. **p<0.01 by Student's t tests. FIG. 11C depicts the Western blot analysis of endogenous ATIC levels by immunoblotting with anti-ATIC antibody. ATIC protein level was completely depleted in ATIC KO SH-SY5Y cells.

FIGS. 12A-12C depict the time- and dose-courses of AICAr suppression on LRRK2 levels. FIG. 12A depicts the endogenous LRRK2 levels upon AICAr treatment at different concentrations. SH-SY5Y cells were treated with 0, 0.01, 0.1, 0.25, 0.5, 1, 2 mM AICAr for 48 hours. Endogenous LRRK2 levels were analyzed by Western blot. FIG. 12B depicts the overexpressed LRRK2 levels upon AICAr treatment at different concentrations. HEK 293T cells were transfected with MYC-LRRK2 and treated with 0, 0.01, 0.1, 0.25, 0.5, 1 mM AICAr for 48 hours. Overexpressed LRRK2 levels were analyzed by Western blot. Quantification of images by ImageJ software. Data are mean±SEM, n=3 independent experiments. **p<0.01, ****p<0.0001 by Student's t tests. FIG. 12C depicts the endogenous LRRK2 levels upon AICAr treatment in a time course. SH-SY5Y cells treated with or without 1 mM AICAr. Cells were collected at different time points. Endogenous LRRK2 levels were analyzed by Western blot.

FIGS. 13A-13E depict that the protein levels of other PD-associated proteins upon AICAr treatment. FIG. 13A depicts the protein level of α-Synuclein upon AICAr treatment. FIG. 13A depicts the protein level of α-Synuclein upon AICAr treatment. FIG. 13B depicts the protein level of VPS35 upon AICAr treatment. FIG. 13C depicts the protein level of Parkin upon AICAr treatment. FIG. 13D depicts the protein level of PINK upon AICAr treatment. FIG. 13E depicts the protein level of DJ-1 upon AICAr treatment. HEK 293T cells were transfected with V5 or flag tagged α-Synuclein, VPS35, Parkin, PINK and DJ-1 and treated with or without 1 mM AICAr for 48 hours. Protein levels were analyzed by Western blot.

FIGS. 14A-14B depict the generation of AUF1 KO and AMPK KO SH-SY5Y cells using the CRISPR/Cas9 editing system. FIG. 14A depicts the Western blot analysis of endogenous AUF1 levels by immunoblotting with anti-AUF1 antibody in SH-SY5Y cells. AUF1 protein level was completely depleted in AUF1 KO SH-SY5Y cells. FIG. 14B depicts the Western blot analysis of endogenous AMPK levels by immunoblotting with anti-AMPK antibody in SH-SY5Y cells. AMPK protein level was completely depleted in AMPK KO SH-SY5Y cells.

FIGS. 15A-15B depict purification of AUF1 isoforms and DCP1A and DCP2. FIG. 15A depicts that recombinant His-tagged AUF1 four isoforms were purified from bacteria. Recombinant his-tagged AUF1 four isoforms were expressed in E. coli BL21 and purified using Ni-NTA agarose. Recombinant His-AUF1-p37/40/42/45 proteins were subjected SDS-PAGE and Coomassie blue staining. FIG. 15B depicts that recombinant Flag-tagged DCP1A and DCP2 were purified from HEK 293T cells. Flag-tagged DCP1A and DCP2 were transfected into HEK 293T cells for 48 hours. Recombinant Flag-tagged DCP1A and DCP2 were purified by immunoprecipitation using anti-Flag antibody. Purified recombinant Flag-tagged DCP1A and DCP2 proteins were analyzed by Western blot.

### DETAILED DESCRIPTION OF THE INVENTION

The present invention is based, at least in part, on the discovery and generation of novel regulators of LRRK2 expression. In particular, the present inventors surprisingly identified 5-aminoimidazole-4-carboxamide ribonucleotide formyltransferase/IMP cyclohydrolase (ATIC), the last enzyme in the de novo purine biosynthesis pathway, as a novel regulator that regulates LRRK2 expression levels and toxicity. In addition, the present inventors also discovered that both AICAr, the precursor of ATIC substrate AICAR/ZMP, and AICAR/ZMP (5-aminoimidazole-4-carboxamide ribonucleotide) itself, can regulate LRRK2 expression levels through an mRNA decay pathway. Furthermore, the inventors discovered that neurodegeneration and inflammation, for example, associated with Parkinson's Disease, can be treated by modulating AICAr and/or AICAR/ZMP mediated-LRRK2 expression. Thus, the present invention provides a novel regulatory mechanism of regulating LRRK2 through LRRK2 mRNA decay and establishes the modulation of LRRK2 mRNA decay as a potential novel therapeutic strategy for LRRK2 associated disorders and conditions, such as Parkinson's Disease, which is distinct from targeting enzymatic functions of LRRK2.

### I. Definitions

In order that the present invention may be more readily understood, certain terms are first defined. In addition, it should be noted that whenever a value or range of values of a parameter are recited, it is intended that values and ranges intermediate to the recited values are also intended to be part of this invention.

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including, but not limited to”.

The term “or” is used herein to mean, and is used interchangeably with, the term “and/or,” unless context clearly indicates otherwise.

The term “about” is used herein to mean within the typical ranges of tolerances in the art, e.g., acceptable variation in time between doses, acceptable variation in dosage unit amount. For example, “about” can be understood as within about 2 standard deviations from the mean. In certain embodiments, about means+10%. In certain embodiments, about means+5%. When about is present before a series of numbers or a range, it is understood that “about” can modify each of the numbers in the series or range.

The term “at least” prior to a number or series of numbers is understood to include the number adjacent to the term “at least”, and all subsequent numbers or integers that could logically be included, as clear from context. For example, the number of nucleotides in a nucleic acid molecule must be an integer. For example, “at least 18 nucleotides of a 21-nucleotide nucleic acid molecule” means that 18, 19, 20, or 21 nucleotides have the indicated property. When at least is present before a series of numbers or a range, it is understood that “at least” can modify each of the numbers in the series or range.

As used herein, “no more than” or “less than” is understood as the value adjacent to the phrase and logical lower values or integers, as logical from context, to zero. For example, an oligonucleotide with “no more than 5 unmodified nucleotides” has 5, 4, 3, 2, 1, or 0 unmodified nucleotides. When “no more than” is present before a series of numbers or a range, it is understood that “no more than” can modify each of the numbers in the series or range.

As used herein, a “leucine rich repeat kinase 2” or “LRRK2” refers to the well-known gene and protein. LRRK2 is also known as dardarin, Parkinson Disease (Autosomal dominant)8, PARK8, AURA17, RIPK7, and ROCO2. LRRK2 is a highly conserved large 286-kDa protein that contains multiple, independent domains belonging to the ROCO protein family. Specifically, the LRRK2 protein contains two enzymatic domains, a GTPase and a kinase domain, and multiple protein-protein interaction domains including a leucine-rich repeat (LRR), a WD40 repeat, and a LRRK2 specific repeat domain. LRRK2 interaction domains are thought to serve as protein binding modules where LRRK2 acts as a signaling scaffold. LRRK2 GTPase and kinase enzyme activity are important in regulating LRRK2 dependent cellular signaling pathways and may reciprocally regulate each other to direct LRRK2's ultimate function. Several mutations of this gene have been demonstrated to be pathogenic. Established dominant inherited Parkinson's Disease (PD)-associated mutations include G2019S, R1441C/G/H, Y1699C, 12020T and N1437H (Bardien S, et al., Parkinsonism Relat Disord. 2011, 17: 501-508). The most common mutation of LRRK2 gene is G2019S, accounting for 5-6% of familial PD, and in 1-2% of sporadic cases of PD (Bardien S, et al., Parkinsonism Relat Disord. 2011, 17: 501-508). People carrying one copy of mutated LRRK2 have a 30% chance of developing PD, which increases sharply with age. Non-familial PD patients also often carry a mutation that arose de novo.

The sequence of a human LRRK2 mRNA transcript can be found at National Center for Biotechnology Information (NCBI) RefSeq accession number NM_198578.4. Additional examples of LRRK2 mRNA sequences are readily available using publicly available databases, e.g., GenBank, UniProt, and OMIM.

An “LRRK2-associated disorder or condition,” as used herein, is intended to include any disease or condition associated with the LRRK2 gene or protein. Such a disease may be caused, for example, by LRRK2 gene mutations, by excess production of the LRRK2 protein, by abnormal cleavage of the LRRK2 protein, by abnormal interactions between LRRK2 and other proteins or other endogenous or exogenous substances.

In some embodiments, the LRRK2-associated disorder or condition is a neurodegenerative disease, such as Parkinson's disease (PD), Alzheimer's disease (AD), dementia (including Lewy body dementia and cascular dementia), amyotrophic lateral sclerosis (ALS), age related memory dysfunction, mild cognitive impairment (e.g., including the transition from mild cognitive impairment to Alzheimer's disease), argyrophilic grain disease, lysosomal disorders (for example, Niemann-PickType C disease, Gaucher disease) corticobasal degeneration, progressive supranuclear palsy, inherited frontotemporal dementia and parkinsonism linked to chromosome 17 (FTDP-17), withdrawal symptoms/relapse associated with drug addiction, L-Dopa induced dyskinesia, Huntington's disease (HD), and HIV-associated dementia (HAD).

In some embodiments, the “LRRK2-associated disorder or condition” is Parkinson's Disease. Clinical symptoms of Parkinson's Disease include akinesia, resting tremor, muscle rigidity, and postural imbalance. In the early stages of the disease, there may be only slight disturbances of posture, locomotion, facial expressions, or speech. Later on, movement difficulty may progress to complete lack of mobility. Symptoms may initially manifest as asymmetric, however as the disease progresses, the symptoms become bilateral and progressively debilitating. PD patients also commonly experience dementia, ataxia, dysphasia, cognitive loss and mood disorders, and the quality and life expectancy of patients with PD is substantially reduced. The neuropathological hallmarks are characterized by a progressive loss of dopaminergic neurons in the substantia nigra pars compacta and the presence of proteinaceous inclusions immunoreactive for α-synuclein termed as Lewy bodies and dystrophic Lewy neurites in surviving neurons.

In some embodiments, the LRRK2-associated disorder or condition is cancer. In certain specific embodiments, the cancer is thyroid, renal (including papillary renal), breast, lung, blood, and prostate cancers (e.g. solid tumor), leukemias (including acute myelogenous leukemia (AML)), or lymphomas. In some embodiments, the cancer is kidney cancer, breast cancer, prostate cancer, blood cancer, papillary cancer, lung cancer, acute myelogenous leukemia, or multiple myeloma.

In some embodiments, the LRRK2-associated disorder or condition is inflammatory disorders. In some embodiments, the LRRK2-associated disorder or condition is an inflammatory disease of the intestines, such as Crohn's disease or ulcerative colitis (both generally known together as inflammatory bowel disease). In some embodiments, the inflammatory disease is leprosy, Crohn's disease, inflammatory bowel disease, ulcerative colitis, amyotrophic lateral sclerosis, rheumatoid arthritis, or ankylosing spondylitis.

As used herein, the term “an agent that decreases the expression of LRRK2” refers to an agent that either directly or indirectly interferes with the expression of LRRK2. In some embodiments, the agent induces LRRK2 mRNA decay. In some embodiments, the agent induces AU-rich element (ARE)-mediated decay.

As used herein, the term “mRNA decay” refers to the process of mRNA degradation or mRNA turnover. mRNA decay is an essential step in gene expression to set mRNA abundance in the cytoplasm. The binding of proteins and/or noncoding RNAs to specific recognition sequences or secondary structures within mRNAs dictates mRNA decay rates by recruiting specific enzyme complexes that perform the destruction processes.

The term “AU-rich element (ARE)-mediated decay (AMD)”, as used herein, refers to a mechanism leading to the rapid degradation of mRNA due to the presence of AU-rich elements (AREs) in their 3′ untranslated regions (3′UTRs). AMD involves exonucleolytic removal of the poly(A) tail, followed by degradation of the mRNA body, either by decapping and 5′ to 3′ degradation or by continued exonucleolytic degradation in the 3′ to 5′.

The term “AU-rich element” or “ARE” refers to an adenylate uridylate rich element in the 3′UTR of an mRNA. AREs can modulate mRNA stability and/or translation depending on the particular ARE-binding proteins (AUBPs) associated. AREs vary widely in sequence but were originally classified into three broad types. Class I AREs, found in mRNAs such as FOS and MYC, contain 1-3 interspersed copies of the AUUUA pentamer surrounded by U-rich regions. Class II AREs, found in cytokine mRNAs such as GM-CSF and tumor necrosis factor α (TNFα), contain multiple overlapping copies (typically 5-8) of the AUUUA motif. Class III AREs, such as the one in JUN mRNA, lack AUUUA pentamers but contain predominantly U-rich sequence. These unique features of AREs suggest that different AUBPs, or more likely, combinations of AUBPs, may differentially regulate the classes of AREs. AUF1 is an AUBP that promotes mRNA degradation.

As used herein, the term “AU-rich element RNA binding protein 1 (AUF)”, also known as, heterogeneous nuclear ribonucleoprotein D; hnRNP D; RNA-binding protein 1, 37 kD; or P37, refers to a member of the subfamily of ubiquitously expressed heterogeneous nuclear ribonucleoproteins (hnRNPs). The hnRNPs are nucleic acid binding proteins and they complex with heterogeneous nuclear RNA (hnRNA). These proteins are associated with pre-mRNAs in the nucleus and appear to influence pre-mRNA processing and other aspects of mRNA metabolism and transport. AUF1 is known to bind with high affinity to RNA molecules that contain AU-rich elements (AREs) and regulates subsequent stability, translation, or subcellular localization of the target mRNA. The sequence of a human AUF1 mRNA transcript can be found at National Center for Biotechnology Information (NCBI) RefSeq accession number NM_031370.3. Additional examples of AUF1 mRNA sequences are readily available using publicly available databases, e.g., GenBank, UniProt, and OMIM.

As used herein the terms “decapping enzyme”, “DCP1” or “DCP2” refer to enzymes that function in removing the 5′ cap from mRNAs, which is a step in mRNA decay. The sequence of a human DCP1 mRNA transcript can be found at National Center for Biotechnology Information (NCBI) RefSeq accession number NM_018403.7 or NM_152640.5. The sequence of a human DCP2 mRNA transcript can be found at National Center for Biotechnology Information (NCBI) RefSeq accession number NM_152624.6. Additional examples of DCP1 or DCP2 mRNA sequences are readily available using publicly available databases, e.g., GenBank, UniProt, and OMIM.

As used herein, the terms “aminoimidazole-4-carboxamide ribonucleotide formyltransferase/IMP cyclohydrolase” or “ATIC”, also known as AICAR transformylase/inosine monophosphate cyclohydrolase, AICARFT, phosphoribosylaminoimidazolecarboxamide formyltransferase/IMP cyclohydrolase, IMPCHASE, or PURH, refer to the enzyme that catalyzes the last two steps of purine de novo synthesis. ATIC metabolizes 5-aminoimidazole-4-carboxamide ribonucleoside (AICAR), which is an AMP analogue, leading to activation of AMP-activated kinase (AMPK). The sequence of a human ATIC mRNA transcript can be found at National Center for Biotechnology Information (NCBI) RefSeq accession number NM_004044.7. Additional examples of ATIC mRNA sequences are readily available using publicly available databases, e.g., GenBank, UniProt, and OMIM.

As used herein, the terms “5-aminoimidazole-4-carboxamide ribonucleotide” or “AICAR”, also known as “acadesine”, is a naturally occurring analogue of adenosine that is taken up by muscle and liver. AICAR is the enzymatic substrate of ATIC. AICAR is phosphorylated to form 5-aminoimidazole-4-carboxamide-1-β-D-ribofuranosyl-5′-monophosphate (ZMP), which activates both AMP-activated protein kinase (AMPK) and AMPK kinase (AMPKK). Treatment with AICAR has been shown to prevent and/or reverse metabolic syndrome in animal models. In ob/ob mice, fa/fa rats, as well as rats fed a high-fat diet, AICAR treatment has been shown to improve glucose tolerance and whole-body glucose disposal as well as reduce hepatic glucose output and plasma triglycerides and free fatty acids levels (Song X. et al., Diabetologia 45, 56-65).

As used herein, the terms “5-aminoimidazole-4-carboxamide riboside” or “AICAr” refer to a cell-permeable AICAR/ZMP precursor AICA riboside (AICAr), a non-monophosphate form of AICAR/ZMP, which is transported into cells through adenosine transporter (AT) and then converted into 5′-monophosphate AICAR/ZMP by adenosine kinase (ADK).

The term “contacting,” as used herein, includes contacting a target gene, e.g., LRRK2, by any means. In some embodiments, a cell is contacted with an agent that decreases the expression of LRRK2, e.g., an agent that induces LRRK2 mRNA decay. Contacting an LRRK2 polynucleotide in a cell with an agent that decreases the expression of LRRK2, e.g., an agent that induces LRRK2 mRNA decay, includes contacting the cell in vitro with the agent, or contacting the cell in vivo with the agent.

Contacting a cell in vitro may be done, for example, by incubating the cell with an agent that decreases the expression of LRRK2, e.g., an agent that induces LRRK2 mRNA decay. Contacting a cell in vivo may be done, for example, by injecting the agent into or near the tissue where the cell is located, or by injecting the agent into another area, e.g., the bloodstream or the subcutaneous space or the brain, such that the agent will subsequently reach the tissue where the cell to be contacted is located. For example, the agent may contain and/or be coupled to a ligand that directs the agent to a site of interest. Combinations of in vitro and in vivo methods of contacting are also possible. For example, a cell may also be contacted in vitro with an agent that decreases the expression of LRRK2, e.g., an agent that induces LRRK2 mRNA decay, and subsequently transplanted into a subject.

In one embodiment, contacting a cell with an agent that decreases the expression of LRRK2, e.g., an agent that induces LRRK2 mRNA decay, includes “introducing” or “delivering the agent into the cell” by facilitating or effecting uptake or absorption into the cell. Absorption or uptake of an agent that decreases the expression of LRRK2, e.g., an agent that induces LRRK2 mRNA decay, can occur through unaided diffusive or active cellular processes, or by auxiliary agents or devices. Introducing an agent that decreases the expression of LRRK2, e.g., an agent that induces LRRK2 mRNA decay, into a cell may be in vitro and/or in vivo. For example, for in vivo introduction, agents can be injected into a tissue site or administered systemically. In vitro introduction into a cell includes methods known in the art such as electroporation and lipofection. Further approaches are described herein below and/or are known in the art.

As used herein, a “subject” is an animal, such as a mammal, including a primate (such as a human, a non-human primate, e.g., a monkey, and a chimpanzee), a non-primate (such as a cow, a pig, a horse, a goat, a rabbit, a sheep, a hamster, a guinea pig, a cat, a dog, a rat, or a mouse), or a bird. A subject may seek or be in need of treatment, require treatment, be receiving treatment, be receiving treatment in the future, or be a human or animal who is under care by a trained professional for a particular disease or condition. In one embodiment, the subject is a mammal. In another embodiment, the subject is a human, such as a human being treated or assessed for an LRRK2-associated disorder or condition, e.g., Parkinson's disease, that would benefit from reduction in LRRK2 expression; a human at risk for developing an LRRK2-associated disorder or condition, e.g., Parkinson's disease, that would benefit from reduction in LRRK2 expression; a human having an LRRK2-associated disorder or condition, e.g., Parkinson's disease, that would benefit from reduction in LRRK2 expression; or human being treated for an LRRK2-associated disorder or condition, e.g., Parkinson's disease, that would benefit from reduction in LRRK2 expression. In some embodiments, the subject is a female human. In other embodiments, the subject is a male human. In one embodiment, the subject is an adult subject. In another embodiment, the subject is an elderly subject.

As used herein, the terms “treat,” “treated,” “treatment” or “treating” refer to measures taken to prevent or slow down (lessen) an undesired physiological condition, disorder, or disease, or obtain beneficial or desired clinical results. Beneficial or desired clinical results include, but are not limited to, alleviation of symptoms of a disease or disorder in a subject, for example, an LRRK2-associated disorder or condition, e.g., Parkinson's Disease; diminishment of the extent of an LRRK2-associated disorder or condition, e.g., Parkinson's Disease; stabilized (i.e., not worsening) state of an LRRK2-associated disorder or condition, e.g., Parkinson's Disease; delay in onset or slowing the progression of an LRRK2-associated disorder or condition, e.g., Parkinson's Disease; amelioration of an LRRK2-associated disorder or condition, e.g., Parkinson's Disease, or remission (whether partial or total), whether detectable or undetectable; an amelioration of at least one measurable physical parameter, not necessarily discernible by the patient; or enhancement or improvement of an LRRK2-associated disorder or condition, e.g., Parkinson's Disease. Treatment includes eliciting a clinically significant response without excessive levels of side effects. Treatment also includes prolonging survival as compared to expected survival if not receiving treatment.

The terms “decreased”, “lower”, or “reduced”, or related forms of such terms, in the context of the expression of LRRK2 in a subject or a disease marker or symptom refers to a statistically significant decrease in such expression. The decrease can be, for example, at least 10%, 15%, 20%, 25%, 30%, %, 40%, 45%, 50%, 55%, 60%, 65%, 70%, 75%, 80%, 85%, 90%, 95%, or more. In certain embodiments, a decrease is at least 20%. In certain embodiments, the decrease is at least 50% in a disease marker, e.g., gene expression level.

As used herein, the term “administration” refers to the administration of a composition (e.g., a compound or a preparation that includes a compound as described herein) to a subject or system. Administration to an animal subject (e.g., to a human) may be by any appropriate route, such as the one described herein.

As used herein, the term “effective amount” refers to the amount of a therapy, which is sufficient to reduce or ameliorate the severity and/or duration of a disorder or one or more symptoms thereof, inhibit or prevent the advancement of a disorder, cause regression of a disorder, inhibit or prevent the recurrence, development, onset or progression of one or more symptoms associated with a disorder, detect a disorder, or enhance or improve the prophylactic or therapeutic effect(s) of another therapy (e.g., prophylactic or therapeutic agent). An effective amount can require more than one dose.

As used herein, a “combination therapy” or “administered in combination” means that two (or more) different agents or treatments are administered to a subject as part of a defined treatment regimen for a particular disease or condition. The treatment regimen defines the doses and periodicity of administration of each agent such that the effects of the separate agents on the subject overlap. In some embodiments, the delivery of the two or more agents is simultaneous or concurrent and the agents may be co-formulated. In some embodiments, the two or more agents are not co-formulated and are administered in a sequential manner as part of a prescribed regimen. In some embodiments, administration of two or more agents or treatments in combination is such that the reduction in a symptom, or other parameter related to the disorder is greater than what would be observed with one agent or treatment delivered alone or in the absence of the other. The effect of the two treatments can be partially additive, wholly additive, or greater than additive (e.g., synergistic). Sequential or substantially simultaneous administration of each therapeutic agent can be effected by any appropriate route including, but not limited to, oral routes, intravenous routes, intramuscular routes, and direct absorption through mucous membrane tissues. The therapeutic agents can be administered by the same route or by different routes. For example, a first therapeutic agent of the combination may be administered by intravenous injection while a second therapeutic agent of the combination may be administered orally.

### II. Methods of the Invention

The present invention is based, at least in part, on the discovery and generation of novel regulators of LRRK2 expression. In particular, the present inventors provides a novel regulatory mechanism of regulating LRRK2 through LRRK2 mRNA decay and establishes the modulation of LRRK2 mRNA decay as a potential novel therapeutic strategy for LRRK2-associated disorder or condition, e.g., Parkinson's disease, which is distinct from targeting enzymatic functions of LRRK2.

Accordingly, in one aspect, the present invention provides methods for reducing LRRK2 expression in a cell. The methods comprise contacting the cell with an agent that decreases the expression of LRRK2, e.g., an agent that induces LRRK2 mRNA decay, thereby reducing LRRK2 expression in the cell.

Gene expression can be regulated on both protein or mRNA levels. The amount of protein generated from any given mRNA depends not only on the rate of mRNA translation but also on the rates of mRNA synthesis and decay. mRNA decay can be divided broadly into two classes: mechanisms of quality control that eliminate the production of potentially toxic proteins, and mechanisms that lengthen or shorten mRNA half-life for the purpose of changing the abundance of functional proteins. The cytoplasmic decay machinery consists of five types of ribonucleolytic activities, the combinatorial and ordered use of which varies depending on the mRNA substrate and cellular conditions. These five activities mediate decapping, 5′-to-3′ exonucleolytic decay, deadenylation, 3′-to-5′ exonucleolytic decay, or endonucleolytic cleavage (Schoenberg, D R, et al., Nat Rev Genet. 2012 Mar. 6; 13(4): 246-259). Each activity may gain access to an mRNA depending on the proteins and, possibly, antisense noncoding (nc)RNA(s) that are bound to the mRNA.

In some embodiments, the agent decreases the expression of LRRK2 by inducing or activating LRRK2 mRNA decay. In some embodiments, the agent decreases the expression of LRRK2 by inducing or activating AU-rich elements (AREs)-mediated mRNA decay. AU-rich elements (AREs) are the largest and most studied group of cis-acting mRNA instability determinants. AREs have been grouped into three broad categories based on the number and context of the AUUUA repeats (Chen C Y, et al. Trends Biochem Sci. 1995; 20:465-470). The decay of ARE-containing mRNAs begins with either synchronous or distributive poly(A) shortening, with the subsequent steps targeting the body of the mRNA from both ends via the actions of the 5′-3′ and 3′-5′ exonucleases (Murray E L, et al., Mol Cell Biol. 2007; 27:2791-2799).

The agents suitable for use in the methods of the present invention include any compound or molecule that can regulate the expression of LRRK2, for example, the mRNA expression or stability of LRRK2. A modulator can modulate the expression LRRK2 either directly or indirectly, e.g., through another molecule, e.g., a binding partner of LRRK2.

In some embodiments, the agent that decreases the expression of LRRK2 is an inhibitor of 5-aminoimidazole-4-carboxamide ribonucleotide formyltransferase/IMP cyclohydrolase (ATIC). In some embodiments, the ATIC inhibitor is selected from the group consisting of a small molecule, an antagonist antibody of ATIC, or antigen-binding fragment thereof, an antisense agent targeting ATIC, a double stranded RNA agent targeting ATIC, an RNA-guided nuclease targeting ATIC, an ATIC fusion protein; and an ATIC inhibitory peptide.

In some embodiments, the agent that decreases the expression of LRRK2 is 5-aminoimidazole-4-carboxamide ribonucleotide (AICAR/ZMP), an enzymatic substrate of ATIC.

In some embodiments, the agent that decreases the expression of LRRK2 is 5-aminoimidazole-4-carboxamide riboside (AICAr), a precursor of ATIC enzymatic substrate AICAR/ZMP.

In some embodiments, the agent that decreases the expression of LRRK2 is an agent that activates or induces LRRK2 mRNA decay. In some embodiments, the agent increases the expression and/or activity of AU-rich element RNA binding protein 1 (AUF1). In some embodiments, the agents include any compound or molecule that can regulate the expression and/or activity of AUF1, for example, the mRNA expression and/or protein expression of AUF1; the mRNA and/or protein stability of AUF1; and/or the biological activity of AUF1. A modulator can modulate the expression and/or activity of AUF1 either directly or indirectly, e.g., through another molecules, e.g., a binding partner of AUF1.

In some embodiments, the agent that increases the expression and/or activity of AUF1 is selected from the group consisting of a small molecule activator of AUF1, an agonist antibody of AUF1, or antigen-binding fragment thereof, an AUF1 protein or functional fragment thereof, a nucleic acid encoding the AUF1 protein or functional fragment thereof, or a protein and a nucleic acid that activates the transcription and/or translation of AUF1.

In some embodiments, the agent that decreases the expression of LRRK2 is an agent that increases the expression and/or activity of a decapping enzyme involved in the process of mRNA decay, e.g., mRNA decapping enzyme 1 (DCP1) and/or mRNA decapping enzyme 2 (DCP2). In some embodiments, the agents include any compound or molecule that can regulate the expression and/or activity of DCP1 and/or DCP2, for example, the mRNA expression and/or protein expression of DCP1 and/or DCP2; the mRNA and/or protein stability of DCP1 and/or DCP2; and/or the biological activity of DCP1 and/or DCP2. A modulator can modulate the expression and/or activity of DCP1 and/or DCP2 either directly or indirectly, e.g., through another molecules, e.g., a binding partner of DCP1 and/or DCP2.

In some embodiments, the agent that increases the expression and/or activity of DCP1 and/or DCP2 is selected from the group consisting of a small molecule activator of DCP1 and/or DCP2, an agonist antibody of DCP1 and/or DCP2, or antigen-binding fragment thereof, a DCP1 and/or DCP2 protein or functional fragment thereof, a nucleic acid encoding the AUF1 protein or functional fragment thereof, or a protein and a nucleic acid that activates the transcription and/or translation of AUF1.

Contacting a cell with an agent includes contacting a cell in vitro with the agent or contacting a cell in vivo with the agent. The contacting may be done directly or indirectly. Thus, for example, the agent may be put into physical contact with the cell by the individual performing the method, or alternatively, the agent may be put into a situation that will permit or cause it to subsequently come into contact with the cell.

Contacting a cell in vitro may be done, for example, by incubating the cell with the agent. Contacting a cell in vivo may be done, for example, by injecting the agent into or near the tissue where the cell is located, or by injecting the agent into another area, e.g., the brain, the bloodstream, or the subcutaneous space, such that the agent will subsequently reach the tissue where the cell to be contacted is located. For example, the agent may contain or be coupled to a ligand that directs the agent to a site of interest. Combinations of in vitro and in vivo methods of contacting are also possible. For example, a cell may also be contacted in vitro with an agent and subsequently transplanted into a subject.

In certain embodiments, contacting a cell with an agent includes “introducing” or “delivering the agent into the cell” by facilitating or effecting uptake or absorption into the cell. Absorption or uptake of an agent can occur through unaided diffusion or active cellular processes, or by auxiliary agents or devices. Introducing an agent into a cell may be in vitro or in vivo. For example, for in vivo introduction, agent can be injected into a tissue site or administered systemically. The agent can also be injected locally. Alternatively, the agent can be applied directly to the target site, e.g., the brain tissue. In vitro introduction into a cell includes methods known in the art such as electroporation and lipofection. Further approaches are described herein below or are known in the art.

The methods of the present invention can be used with cells from any organ, e.g., brain, skin, lung, heart, kidney, liver, pancreas, gut, muscle, gland, eye, blood and the like. The invention is particularly suitable for modifying sequences in cells, tissues or organs implicated in a diseased state of a (human) subject. Such cells include, but are not limited to, brain cells or neurons, e.g., neurons near the base of the brain (i.e., midbrain or the substantia nigra), neurons from the brainstem, neurons in the striatum, neurons in the cortex, or neurons in the olfactory bulb. Cells outside the brain can also be included.

In some embodiments, the cells are neurons, microglial cells, or fibroblasts.

In some embodiments, the cells are within a subject, e.g., a human subject. In some embodiments, the subject has an LRRK2-associated disorder or condition, e.g., Parkinson's Disease.

In another aspect, the present invention provides methods for treating an LRRK2-associated disorder or condition, e.g., Parkinson's Disease, in a subject in need thereof, methods for reducing or preventing neuronal cell death in a subject in need thereof, and method of reducing neurodegeneration and/or neuroinflammation in a subject in need thereof. In further embodiments, the invention provides methods for identifying a compound useful for treating an LRRK2-associated disorder or condition, e.g., Parkinson's Disease.

The methods comprise administering to the subject an effective amount of an agent that decreases the expression of LRRK2. In some embodiments, the agent induces or activates LRRK2 mRNA decay. In some embodiments, the agent does not modulate the enzymatic activity of LRRK2.

The methods are suitable for treating or preventing LRRK2-associated disorders or conditions. As used herein, the term “LRRK2-associated disorder or condition” is intended to include any disease or condition associated with the LRRK2 gene or protein. Such a disease may be caused, for example, by LRRK2 gene mutations, by excess production of the LRRK2 protein, by abnormal cleavage of the LRRK2 protein, by abnormal interactions between LRRK2 and other proteins or other endogenous or exogenous substances.

LRRK2 has been associated with the transition from mild cognitive impairment to Alzheimer's disease; L-Dopa induced dyskinesia (Hurley et al., Eur. J, Neurosci., Vol. 26, 2007, 171-177); central nervous system (CNS) disorders associated with neuroprogenitor cell proliferation and migration, and regulation of LRRK2 may have utility in improving neurological outcomes following ischemic injury, and stimulating restoration of CNS function following neuronal injury such as ischemic stroke, traumatic brain injury, or spinal cord injury (Milosevic et al., Neurodegen., Vol. 4, 2009, 25; See Zhang et al., J. Neurosci. Res. Vol. 88, 2010, 3275-3281); Parkinson's disease, Alzheimer's disease, multiple sclerosis, and HIV-induced dementia (See Milosevic et al., Mol. Neurodegen., Vol. 4, 2009, 25); kidney, breast, prostate (e.g. solid tumor), blood and lung cancer, and acute myeologenouse leukemia (AML); lymphomas and leukemias (See Ray et al., J. Immunolo., Vol. 230, 2011, 109); multiple myeoloma (Chapman et al., Nature, Vol. 471, 2011, 467-472); papillary renal and thyroid carcinomas; multiple myeloma (Chapman et al., Nature, Vol. 471, 2011, 467-472); diseases of the immune system, including rheumatoid arthritis, systemic lupus erythematosus autoimmune hemolytic anemia, pure red cell aplasia, idiopathic thrombocytopenic pupura (ITP), Evans syndrome, vasculitis, bullous skin disorders, type 1 diabetes mellitus, Sjogren's syndrome, Delvic's disease, and inflammatory myopathies (Nakamura et al., DNA Res. Vol. 13(4), 2006, 169-183; See Engel et al., Pharmacol. Rev. Vol. 63, 2011, 127-156; Homam et al., J. Clin. Neuromuscular Disease, Vol. 12, 2010, 91-102); ankylosing spondylitis and leprosy infection (DAnoy et al., PLoS Genetics, Vol. 6(12), 2010, e1001195, 1-5; see Zhang et al., N. Eng. J. Med. Vol. 361, 2009, 2609-2618); alpha-synucleinopathies, taupathies (See Li et al., 2010 Neurodegen. Dis. Vol. 7, 2010, 265-271); Gaucher disease (See Westbroek et al., Trends. Mol. Med. Vol. 17, 2011, 485-493); tauopathy diseases characterized by hyperphosphorylation of Tau such as argyrophilic grain disease, Pick's disease, corticobasal degeneration, progressive supranuclear palsy, and inherited frontotemporal dementia and parkinsonism linked to chromosome 17 (See Goedert, M and Jakes, R, Biochemica et Biophysica Acta, Vol. 1739, 2005, 240-250); diseases characterized by diminished dopamine levels such as withdrawal symptoms/relapse associated with drug addiction (See Rothman et al., og. Brain Res., Vol. 172, 2008, 385); microglial proinflammatory responses (See Moehle et al., J. Neuroscience Vol. 32, 2012, 1602-1611); Crohn's disease pathogenesis (see Barrett et al., Nature Genetics, Vol. 40, 2008, 955-962); and amyotrophic lateral sclerosis (ALS).

In some embodiments, the LRRK2-associated disorder or condition is a neurodegenerative disease, such as Parkinson's disease (PD), Alzheimer's disease (AD), dementia (including Lewy body dementia and cascular dementia), amyotrophic lateral sclerosis (ALS), age related memory dysfunction, mild cognitive impairment (e.g., including the transition from mild cognitive impairment to Alzheimer's disease), argyrophilic grain disease, lysosomal disorders (for example, Niemann-PickType C disease, Gaucher disease) corticobasal degeneration, progressive supranuclear palsy, inherited frontotemporal dementia and parkinsonism linked to chromosome 17 (FTDP-17), withdrawal symptoms/relapse associated with drug addiction, L-Dopa induced dyskinesia, Huntington's disease (HD), and HIV-associated dementia (HAD).

In some embodiments, the LRRK2-associated disorder or condition is Parkinson's Disease. Mutations in LRRK2 gene are the most frequent genetic causes of both sporadic and familial Parkinson's disease (PD). Clinical symptoms of Parkinson's Disease include akinesia, resting tremor, muscle rigidity, and postural imbalance. In the early stages of the disease, there may be only slight disturbances of posture, locomotion, facial expressions, or speech. Later on, movement difficulty may progress to complete lack of mobility. Symptoms may initially manifest as asymmetric, however as the disease progresses, the symptoms become bilateral and progressively debilitating. PD patients also commonly experience dementia, ataxia, dysphasia, cognitive loss and mood disorders, and the quality and life expectancy of patients with PD is substantially reduced. The neuropathological hallmarks are characterized by a progressive loss of dopaminergic neurons in the substantia nigra pars compacta and the presence of proteinaceous inclusions immunoreactive for α-synuclein termed as Lewy bodies and dystrophic Lewy neurites in surviving neurons.

In some embodiments, the LRRK2-associated disorder or condition is inflammatory disorders. LRRK2 has been connected genetically to a number of chronic inflammatory conditions, beginning in 2008 with linkage to Crohn's disease, an inflammation of the terminal ileum, which was found via meta-analysis with subsequent replication of three separate GWAS investigations (Barrett J C, et al. Nat Genet. 2008; 40(8):955-62). Crohn's disease is one of two distinct chronic inflammatory intestinal disorders that are grouped together as inflammatory bowel disease, the other being ulcerative colitis, an inflammation of the colon. Linkage to LRRK2 was also reported in GWAS of Chinese leprosy patients (Zhang F R, et al. The New Engl J Med. 2009; 361(27):2609-18). Leprosy (also known as Hansen's disease) is a chronic inflammatory condition caused by Mycobacterium leprae infection of the skin and peripheral nerves.

In some embodiments, the LRRK2-associated disorder or condition is an inflammatory disease of the intestines, such as Crohn's disease or ulcerative colitis (both generally known together as inflammatory bowel disease). In some embodiments, the inflammatory disease is leprosy, Crohn's disease, inflammatory bowel disease, ulcerative colitis, amyotrophic lateral sclerosis, rheumatoid arthritis, or ankylosing spondylitis.

In some embodiments, the LRRK2-associated disorder or condition is cancer. Individuals with the most common pathogenic LRRK2 mutation, G2019S, have been reported to have an increased risk of developing cancers (Saunders-Pullman R, et al. Mov Dis. 2010; 25(15):2536-41).

As described herein, the term “cancer” refers to one of a group of diseases caused by the uncontrolled, abnormal proliferation of cells that can spread to adjoining tissues or other parts of the body. Cancer cells can form a solid tumor, in which the cancer cells are massed together, or exist as dispersed cells, as in leukemia. Types of cancer that are suitable to be treated by decreasing the expression or activity of LRRK2 include, but are not limited to, solid tumors and/or hematological cancers. In one embodiment, the cancer is of epithelial origin.

Exemplary types of cancer that can be treated by the foregoing methods include, but are not limited to, adrenal cancer, anal cancer, bile duct cancer, bladder cancer, bone cancer, brain/CNS tumors, breast cancer, castleman disease, cervical cancer, colon/rectum cancer, endometrial cancer, esophagus cancer, eye cancer, gallbladder cancer, gastrointestinal cancer, kidney cancer, laryngeal and hypopharyngeal cancer, leukemia, liver cancer, lung cancer, lymphoma, lymphoma of the skin, malignant mesothelioma, multiple myeloma, myelodysplastic syndrome, nasal cavity and paranasal sinus cancer, nasopharyngeal cancer, neuroblastoma, non-hodgkin lymphoma, oral cavity and oropharyngeal cancer, osteosarcoma, ovarian cancer, pancreatic cancer, penile cancer, pituitary tumors, prostate cancer, retinoblastoma, rhabdomyosarcoma, salivary gland cancer, skin cancer, small intestine cancer, stomach cancer, testicular cancer, thymus cancer, thyroid cancer, uterine sarcoma, vaginal cancer, vulvar cancer, waldenstrom macroglobulinemia, and wilms tumor. In some embodiments, the cancer is selected from the group consisting of brain cancer, lung cancer, pancreatic cancer, melanoma cancer, breast cancer, ovarian cancer, renal cell carcinoma, rectal adenocarcinoma, hepatocellular carcinoma, and Ewing sarcoma.

In some embodiments, the cancer is colon cancer, endometriod cancer, kidney cancer (e.g., kidney papillary cell carcinoma, kidney clear cell carcinoma), liver cancer, thyroid cancer, lung cancer (e.g., lung adenocarcinoma, lung squamous cell carcinoma), head and neck cancer, breast cancer, cervical cancer, prostate cancer, bladder cancer, glioblastoma, rectal cancer, or bile duct cancer. In one embodiment, the cancer is brain cancer. In one embodiment, the cancer is glioblastoma. In one embodiment, the cancer is a blood-derived cancer. In one embodiment, the cancer is leukemia. In one embodiment, the cancer is acute myeloid leukemia (AML).

Pharmaceutical compositions described herein are suitable for administration in human or non-human subjects. Accordingly, agents that decrease the expression of LRRK2, e.g., agents that induce or activate LRRK2 mRNA decay, are useful as medicament for administering to a subject who is likely to benefit from reduced LRRK2 expression.

In some embodiments, suitable subjects have an LRRK2-associated disorder or condition, e.g., Parkinson's Disease. In some embodiments, suitable subjects are at risk of developing an LRRK2-associated disorder or condition, e.g., Parkinson's Disease. In some embodiments, suitable subjects are those on a therapy comprising another therapeutic agent to treat an LRRK2-associated disorder or condition, e.g., Parkinson's Disease, however, these therapies may be associated with adverse effects or high recurrence rates. In some embodiments, suitable subjects include healthy individuals who may nonetheless benefit from reduced LRRK2 expression.

In some embodiments, such medicament is suitable for administration in an adult population, and/or an elderly population.

The population in need for the agents decreasing the expression of LRRK2 (e.g., an ATIC inhibitor, an ATIC substrate (e.g., AICAR/ZMP), or a precursor of ATIC substrate (e.g., AICAr), an agent that increases the expression and/or activity of AUF1, or an agent that increases the expression and/or activity of DCP1 and/or DCP2, described herein) may have an age of at least 40 years, e.g., at least 40, 45, 50, 55, 60 or 65 years. In some embodiments, the population may be below 65 years of age. In some embodiments, the population may be of (a) at least 40 years of age and (b) below 65 years of age. In some embodiments, the population may have an age of 65 years or older (i.e., >65 years old), e.g., at least 70, 75 or 80 years.

A human subject who is likely to benefit from the treatment may be a human patient having, at risk of developing, or suspected of having an LRRK2-associated disorder or condition, e.g., Parkinson's Disease. A subject having cancer can be identified by routine medical examination, e.g., laboratory tests, dopamine transporter (DAT) scan, imaging tests, e.g., PET scans, MRI, or ultrasounds of the brain. A subject suspected of having any of such disease/disorder might show one or more symptoms of the disease/disorder. A subject at risk for the disease/disorder can be a subject having one or more of the risk factors for that disease/disorder.

A control subject, as described herein, is a subject who provides an appropriate reference for evaluating the effects of a particular treatment or intervention of a test subject or subject. Control subjects can be of similar age, race, gender, weight, height, and/or other features, or any combination thereof, to the test subjects.

In some embodiments, the agents that decrease the expression of LRRK2 (e.g., an ATIC inhibitor, an ATIC substrate (e.g., AICAR/ZMP), or a precursor of ATIC substrate (e.g., AICAr), an agent that increases the expression and/or activity of AUF1, or an agent that increases the expression and/or activity of DCP1 and/or DCP2, described herein) are administered to a subject in need of the treatment at an amount sufficient to reduce expression of LRRK2, e.g., LRRK2 mRNA levels, by at least 10% (e.g., 20% 30%, 40%, 50%, 60%, 70%, 80%, 90% or greater) in vivo. In other embodiments, the agents that decrease the expression of LRRK2 (e.g., an ATIC inhibitor, an ATIC substrate, (e.g., AICAR/ZMP), or a precursor of ATIC substrate (e.g., AICAr), an agent that increases the expression and/or activity of AUF1, or an agent that increases the expression and/or activity of DCP1 and/or DCP2), are administered in an amount effective to reduce expression of LRRK2, e.g., LRRK2 mRNA levels, by at least 10% (e.g., 20%, 30%, 40%, 50%, 60%, 70%, 80%, 90% or greater) in vitro.

The methods of the present invention further comprise selecting a subject. In some embodiments, the subject suffers from or is at risk of developing an LRRK2-associated disorder or condition, e.g., Parkinson's Disease.

To practice the method disclosed herein, an effective amount of the pharmaceutical composition described above can be administered to a subject (e.g., a human) in need of the treatment via any suitable route known to those of ordinary skill in the art, such as intravenous administration, e.g., as a bolus or by continuous infusion over a period of time, by intracranial, intrathecal, intracerebrospinal, intramuscular, intraperitoneal, subcutaneous, intra-articular, intrasynovial, intrathecal, oral, inhalation or topical mutes. Commercially available nebulizers for liquid formulations, including jet nebulizers and ultrasonic nebulizers are useful for administration. Liquid formulations can be directly nebulized and lyophilized powder can be nebulized after reconstitution. Alternatively, the agents that decreases the expression of LRRK2 can be aerosolized using a fluorocarbon formulation and a metered dose inhaler, or inhaled as a lyophilized and milled powder.

Conventional methods can be used to administer the pharmaceutical composition to the subject, depending upon the type of disease to be treated or the site of the disease. This composition can also be administered via other conventional mutes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In addition, it can be administered to the subject via injectable depot mutes of administration such as using 1-, 3-, or 6-month depot injectable or biodegradable materials and methods.

Injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the antibody and a physiologically acceptable excipients is infused. Physiologically acceptable excipients may include, for example, 5% dextrose, 0.9% saline, Ringer's solution or other suitable excipients. Intramuscular preparations, e.g., a sterile formulation of a suitable soluble salt form of the antibody, can be dissolved and administered in a pharmaceutical excipient such as Water-for-Injection, 0.9% saline, or 5% glucose solution.

In one embodiment, an agent that decreases the expression of LRRK2 is administered via site-specific or targeted local delivery techniques. Examples of site-specific or targeted local delivery techniques include topical administration, e.g., directly to the brain. In some embodiments, the agent that decreases the expression of LRRK2, e.g., AICAR/ZMP and/or AICAr, is administered via osmotic minipumps by subcutaneous implantation.

The particular dosage regimen, e.g., dose, timing and repetition, used in the method described herein will depend on the particular subject and that subject's medical history.

“An effective amount” as used herein refers to the amount of each active agent required to confer a therapeutic effect on the subject, either alone or in combination with one or more other active agents. For example, an effective amount refers to the amount of an agent decreasing the expression of LRRK2 of the present disclosure which is sufficient to achieve a biological effect.

Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

In some embodiments, in the context of a decrease in the expression level of LRRK2 in a cell, the decrease is at least 1-fold, 1.2-fold, 1.5-fold, 2-fold, 2.5-fold, 3-fold, 4-fold, 5-fold, 6-fold, 7-fold, 8-fold, 9-fold, or 10-fold or more (or any range bracketed by any of the values), compared to a control level of LRRK2. In one embodiment, the decrease in the expression level of LRRK2in the cell is a decrease in a range of 1-fold to 3-fold, 1.2-fold to 10-fold, 2-fold to 9-fold, 3-fold to 8-fold, 4-fold to 7-fold, 2-fold to 7-fold, etc. compared to the control level of LRRK2.

In some embodiments, in the context of a decrease in the expression level of LRRK2 in the cell after the administering step, the decrease is detectable within 4 hours, 24 hours, 48 hours, 7 days, 14 days, 21 days, 28 days or 30 days (or any time range bracketed by any of the listed duration of times) after the administering step. In one embodiment, a decrease in the expression level of LRRK2 in the cell after the administering step is detectable for at least 5 days, 7 days, 14 days, 21 days, 28 days, or 30 days (or any time range bracketed by any of the listed duration of times) after the administering step. In one embodiment, a decrease in the expression level of LRRK2 in the cell after the administering step is at least 1-fold, 1.2-fold, 1.5-fold, 2-fold, 2.5-fold, 3-fold, 4-fold, 5-fold, 6-fold, 7-fold, 8-fold, 9-fold, or 10-fold or more (or any range bracketed by any of the values), compared to the level of LRRK2 in the cell before the administering step. In one embodiment, a decrease in the expression level of LRRK2 in the cell after the administering step is a decrease in a range of 1-fold to 3-fold, 1.2-fold to 10-fold, 2-fold to 9-fold, 3-fold to 8-fold, 4-fold to 7-fold, 2-fold to 7-fold, etc., compared to the level of LRRK2 in the cell before the administering step.

The level of mRNA of a gene of interest, e.g., LRRK2, may be determined using any method known in the art for assessing mRNA expression. In one embodiment, the level of LRRK2 expression in a sample is determined by detecting a transcribed polynucleotide, or portion thereof, e.g., LRRK2 mRNA. RNA may be extracted from cells using RNA extraction techniques including, for example, using acid phenol/guanidine isothiocyanate extraction (RNAzol B; Biogenesis), RNEASY™ RNA preparation kits (Qiagen) or PAXgene (PreAnalytix, Switzerland). Typical assay formats utilizing ribonucleic acid hybridization include nuclear run-on assays, RT-PCR, RNase protection assays, northern blotting, in situ hybridization, and microarray analysis. Circulating mRNA of the gene of interest may be detected using methods the described in PCT Publication WO2012/177906, the entire contents of which are hereby incorporated herein by reference. In some embodiments, the level of LRRK2 expression is determined using a nucleic acid probe. The term “probe,” as used herein, refers to any molecule that is capable of selectively binding to a specific sequence, e.g. to an mRNA or polypeptide. Probes can be synthesized by one of skill in the art, or derived from appropriate biological preparations. Probes may be specifically designed to be labeled. Examples of molecules that can be utilized as probes include, but are not limited to, RNA, DNA, proteins, antibodies, and organic molecules.

Isolated mRNA can be used in hybridization or amplification assays that include, but are not limited to, southern or northern analyses, polymerase chain reaction (PCR) analyses, and probe arrays. One method for the determination of mRNA levels involves contacting the isolated mRNA with a nucleic acid molecule (probe) that can hybridize to the mRNA of a gene of interest, e.g., LRRK2. In one embodiment, the mRNA is immobilized on a solid surface and contacted with a probe, for example by running the isolated mRNA on an agarose gel and transferring the mRNA from the gel to a membrane, such as nitrocellulose. In an alternative embodiment, the probe(s) are immobilized on a solid surface and the mRNA is contacted with the probe(s), for example, in an Affymetrix gene chip array. A skilled artisan can readily adapt known mRNA detection methods for use in determining the level of mRNA of a gene of interest.

An alternative method for determining the level of expression of a gene of interest, e.g., LRRK2, in a sample involves the process of nucleic acid amplification and/or reverse transcriptase (to prepare cDNA) of for example mRNA in the sample, e.g., by RT-PCR (the experimental embodiment set forth in Mullis, 1987, U.S. Pat. No. 4,683,202), ligase chain reaction (Barany (1991) Proc. Natl. Acad. Sci. USA 88:189-193), self-sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules using techniques well known to those of skill in the art. These detection schemes are especially useful for the detection of nucleic acid molecules if such molecules are present in very low numbers. In some embodiments, the level of expression of a gene of interest, e.g., LRRK2, is determined by quantitative fluorogenic RT-PCR (i.e., the TAQMAN™ System) or the DUAL-GLO® Luciferase assay.

The expression levels of mRNA of a gene of interest, e.g., LRRK2, may be monitored using a membrane blot (such as used in hybridization analysis such as northern, Southern, dot, and the like), or microwells, sample tubes, gels, beads or fibers (or any solid support including bound nucleic acids). See U.S. Pat. Nos. 5,770,722; 5,874,219; 5,744,305; 5,677,195; and 5,445,934, which are incorporated herein by reference. The determination of gene expression level may also include using nucleic acid probes in solution.

In some embodiments, the level of mRNA expression is assessed using branched DNA (bDNA) assays or real time PCR (qPCR). The use of this PCR method is described and exemplified in the Examples presented herein. Such methods can also be used for the detection of nucleic acids of the gene of interest.

The level of protein produced by the expression of a gene of interest, e.g., LRRK2, may be determined using any method known in the art for the measurement of protein levels. Such methods include, for example, electrophoresis, capillary electrophoresis, high performance liquid chromatography (HPLC), thin layer chromatography (TLC), hyperdiffusion chromatography, fluid or gel precipitin reactions, absorption spectroscopy, a colorimetric assays, spectrophotometric assays, flow cytometry, immunodiffusion (single or double), immunoelectrophoresis, western blotting, radioimmunoassay (RIA), enzyme-linked immunosorbent assays (ELISAs), immunofluorescent assays, electrochemiluminescence assays, and the like. Such assays can also be used for the detection of proteins indicative of the presence or replication of proteins produced by the gene of interest. Additionally, the above assays may be used to report a change in the mRNA sequence of interest that results in the recovery or change in protein function thereby providing a therapeutic effect and benefit to the subject, treating a disorder in a subject, and/or reducing of symptoms of a disorder in the subject.

### Combination Therapies

A method of the invention can be used alone or in combination with an additional therapeutic agent, e.g., other agents that treat the same disorder, e.g., an LRRK2-associated disorder or condition, e.g., Parkinson's Disease, or symptoms associated therewith, or in combination with other types of therapies to the disorder. In combination treatments, the dosages of one or more of the therapeutic compounds may be reduced from standard dosages when administered alone. For example, doses may be determined empirically from drug combinations and permutations or may be deduced by isobolographic analysis. Dosages of the compounds when combined should provide a therapeutic effect.

In any of these embodiments, such subjects may receive combination therapies that include a first composition comprising at least one agent that decreases the expression of LRRK2, e.g., an ATIC inhibitor, or an ATIC substrate (e.g., AICAR/ZMP), or a precursor of ATIC substrate (e.g., AICAr), as described herein, in conjunction with a second composition comprising at least one additional therapeutic intended to treat the same or overlapping disease or clinical condition. The first and second compositions may both act on the same cellular target, or discrete cellular targets. In some embodiments, the first and second compositions may treat or alleviate the same or overlapping set of symptoms or aspects of a disease or clinical condition. In some embodiments, the first and second compositions may treat or alleviate a separate set of symptoms or aspects of a disease or clinical condition. Such combination therapies may be administered in conjunction with each other. The phrase “in conjunction with,” in the context of combination therapies, means that therapeutic effects of a first therapy overlaps temporarily and/or spatially with therapeutic effects of a second therapy in the subject receiving the combination therapy. Thus, the combination therapies may be formulated as a single formulation for concurrent administration, or as separate formulations, for sequential administration of the therapies.

In preferred embodiments, combination therapies produce synergistic effects in the treatment of a disease. The term “synergistic” refers to effects that are greater than additive effects (e.g., greater efficacy) of each monotherapy in aggregate.

In some embodiments, combination therapies comprising a pharmaceutical composition described herein produce efficacy that is overall equivalent to that produced by another therapy (such as monotherapy of a second agent) but are associated with fewer unwanted adverse effect or less severe toxicity associated with the second agent, as compared to the monotherapy of the second agent. In some embodiments, such combination therapies allow lower dosage of the second agent but maintain overall efficacy. Such combination therapies may be particularly suitable for patient populations where a long-term treatment is warranted and/or involving pediatric patients.

It should be understood that the pharmaceutical compositions described herein may have the first and second therapies in the same pharmaceutically acceptable carrier or in a different pharmaceutically acceptable carrier for each described embodiment. It further should be understood that the first and second therapies may be administered simultaneously or sequentially within described embodiments.

The one or more agents that decreases the expression of LRRK2 of the invention may be used in combination with one or more of additional therapeutic agents. In some embodiments, the additional therapeutic agent is a dopamine precursor. In other embodiments, the additional therapeutic agent is a dopamine receptor agonist. In some embodiments, the additional therapeutic agent is an anticholinergic drug. In some embodiments, the additional therapeutic agent is an interferon, e.g., interferon-gamma, or TNF-alpha.

The additional agent may also be a therapeutic agent which is a non-drug treatment. For example, the additional therapeutic agent is physical therapy, or a psychiatric treatment.

Exemplary drugs used to treat Parkinson's Disease include L-dopa, selegiline, apomorphine, anticholinergics, a dopamine agonist, a monoamine oxidase B (MAO B) inhibitor, a catechol O-methyltransferase (COMT) inhibitor, or an adenosine receptor antagonist. L-dopa (levo-dihydroxy-phenylalanine) (sinemet) is a dopamine precursor which can cross the blood-brain barrier and be converted to dopamine in the brain. It is usually combined with carbidopa, which prevents or reduces potential side effects. Selegiline (Deprenyl, Eldepryl) has been used as an alternative to L-dopa, and acts by reducing the breakdown of dopamine in the brain. Apomorphine, a dopamine receptor agonist, has also been used to treat Parkinson's Disease and it directly stimulates receptors in nerves that would normally be stimulated by dopamine. Systemically administered anticholinergic drugs (such as benzhexol and orphenedrine) have also been used to treat Parkinson's Disease and act by reducing the amount of acetylcholine produced in the brain and thereby redress the dopamine/acetylcholine imbalance present in Parkinson's Disease. Stereotactic surgery offered one of the few effective treatments for Parkinson's Disease.

Examples of the additional therapeutic agents which can be used with an agent of the invention include, may also include nonsteroidal anti-inflammatory drugs (NSAIDS), chemotherapeutic agents, immunotherapeutic agents, immunosuppressive agents, and the like.

Exemplary nonsteroidal anti-inflammatory agents (NSAIDS) include, but are not limited to, aspirin, celecoxib, diclofenac, ibuprofen, indomethacin, ketoprofen, naproxen, oxaprozin, piroxicam.

Chemotherapeutic agents include, for example, alkylating agents (e.g., cyclophosphamide, iphosphamide and the like), metabolism antagonists (e.g., methotrexate, 5-fluorouracil and the like), anticancer antibiotics (e.g., mitomycin, adriamycin and the like), vegetable-derived anticancer agents (e.g., vincristine, vindesine, taxol and the like), cisplatin, carboplatin, etoposide and the like. Among these substances, 5-fluorouracil derivatives such as furtulon and neofurtulon are preferred.

Immunotherapeutic agents include, for example, microorganisms or bacterial components (e.g., muramyl dipeptide derivative, picibanil and the like), polysaccharides having immune potentiating activity (e.g., lentinan, sizofilan, krestin and the like), cytokines obtained by a gene engineering technology (e.g., interferon, interleukin (IL) and the like), colony stimulating factors (e.g., granulocyte colony stimulating factor, erythropoetin and the like) and the like, among these substances, those preferred are IL-1, IL-2, IL-12 and the like.

Immunosuppressive agents include, for example, calcineurin inhibitor/immunophilin modulators such as cyclosporine (Sandimmune, Gengraf, Neoral), tacrolimus (Prograf, FK506), ASM 981, sirolimus (RAPA, rapamycin, Rapamune), or its derivative SDZ-RAD, glucocorticoids (prednisone, prednisolone, methylprednisolone, dexamethasone and the like), purine synthesis inhibitors (mycophenolate mofetil, MMF, CellCept®, azathioprine, cyclophosphamide), interleukin antagonists (basiliximab, daclizumab, deoxyspergualin), lymphocyte-depleting agents such as antithymocyte globulin (Thymoglobulin, Lymphoglobuline), anti-CD3 antibody (OKT3), and the like.

Any of the above-mentioned agents can be administered in combination with the agent that decreases the expression of LRRK2 to treat or prevent an LRRK2-associated disorder or condition, e.g., Parkinson's disease.

### Screening Methods

In certain aspect, the presention provides a method for identifying a compound that modulates, e.g., increase or decrease, the expression and/or activity of LRRK2, AITC, AUF1, DCP1 and/or DPC2. Compounds that are capable of inhibiting the expression, stability, and/or activity of LRRK2, AITC, AUF1, DCP1 and/or DPC2, as identified by the methods of the invention, are useful as candidates to treat LRRK2-associated disorders or conditions, e.g., Parkinson's disease, to reduce or prevent neuronal cell death, or to reduce neurodegeneration and/or neuroinflammation in a subject in need thereof.

Accordingly, in one aspect, the present invention provides methods for identifying a compound useful for treating an LRRK2-associated disorder or condition in a subject, comprising providing a test compound; determining the effect of the test compound on the expression and/or activity of LRRK2, AITC, AUF1, DCP1 and/or DPC2; and selecting a compound which decreases the expression and/or activity of LRRK2 and AITC, or a compound which increases the expression and/or activity of AUF1, DCP1 and/or DCP2, thereby identifying a compound useful for treating a cancer in the subject.

In another aspect, the present invention provides a method of identifying a compound useful for reducing or preventing neuronal cell death in a subject in need thereof, comprising
providing a test compound; determining the effect of the test compound on the expression and/or activity of LRRK2, AITC, AUF1, DCP1 and/or DPC2; and selecting a compound which decreases the expression and/or activity of LRRK2 and AITC, or a compound which increases the expression and/or activity of AUF1, DCP1 and/or DCP2, thereby identifying a compound useful for reducing or preventing neuronal cell death in the subject.

In another aspect, the present invention provides a method of identifying a compound useful for reducing neurodegeneration and/or neuroinflammation in a subject in need thereof, comprising providing a test compound; determining the effect of the test compound on the expression and/or activity of LRRK2, AITC, AUF1, DCP1 and/or DPC2; and selecting a compound which decreases the expression and/or activity of LRRK2 and AITC, or a compound which increases the expression and/or activity of AUF1, DCP1 and/or DCP2, thereby identifying a compound useful for reducing neurodegeneration and/or neuroinflammation in the subject.

Examples of modulators, candidate compounds or test compounds include, but are not limited to, nucleic acids (e.g., DNA and RNA), carbohydrates, lipids, proteins, peptides, peptidomimetics, small molecules and other drugs. Modulators can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds (Lam (1997) Anticancer Drug Des. 12:145; U.S. Pat. Nos. 5,738,996; and 5,807,683, the entire contents of each of the foregoing references are incorporated herein by reference).

Examples of methods for the synthesis of molecular libraries can be found in the art, for example in: DeWitt et al. (1993) Proc. Natl. Acad. Sci. USA 90:6909; Erb et al. (1994) Proc. Natl. Acad. Sci. USA 91:11422; Zuckermann et al. (1994) J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and Gallop et al. (1994) J. Med. Chem. 37:1233, the entire contents of each of the foregoing references are incorporated herein by reference. Libraries of compounds may be presented, e.g., presented in solution (e.g., Houghten (1992) Bio/Techniques 13:412-421), or on beads (Lam (1991) Nature 354:82-84), chips (Fodor (1993) Nature 364:555-556), bacteria (U.S. Pat. No. 5,223,409), spores (U.S. Pat. Nos. 5,571,698; 5,403,484; and 5,223,409), plasmids (Cull et al. (1992) Proc. Natl. Acad. Sci. USA 89:1865-1869) or phage (Scott and Smith (19900 Science 249:386-390; Devlin (1990) Science 249:404-406; Cwirla et al. (1990) Proc. Natl. Acad. Sci. USA 87:6378-6382; and Felici (1991) J. Mol. Biol. 222:301-310). The entire contents of each of the foregoing references are incorporated herein by reference.

The test compound can be contacted with a cell that expresses the target protein, e.g., LRRK2, AITC, AUF1, DCP1 and/or DPC2, or a molecule with which LRRK2, AITC, AUF1, DCP1 and/or DPC2 directly interacts. For example, the test compound can be contacted with a cell that naturally expresses or has been engineered to express the protein(s) by introducing into the cell an expression vector encoding the protein.

Alternatively, the test compounds can be subjected to a cell-free composition that includes the protein(s) (e.g., a cell extract or a composition that includes e.g., purified natural or recombinant protein).

Compounds that modulate expression and/or activity of LRRK2, AITC, AUF1, DCP1 and/or DPC2, or a binding partner of LRRK2, AITC, AUF1, DCP1 and/or DPC2, can be identified using various “read-outs.” For example, a cell can be transfected with an expression vector, incubated in the presence and in the absence of a test compound, and the effect of the compound on the expression of LRRK2, AITC, AUF1, DCP1 and/or DPC2 or on a biological response regulated by LRRK2, AITC, AUF1, DCP1 and/or DPC2 can be determined. The biological activities of LRRK2, AITC, AUF1, DCP1 and/or DPC2 include activities determined in vivo, or in vitro, according to standard techniques.

To determine whether a test compound modulates protein expression, in vitro transcriptional assays can be performed. To determine whether a test compound modulates mRNA expression, various methodologies can be performed, such as quantitative or real-time PCR.

A variety of reporter genes are known in the art and are suitable for use in the screening assays of the invention. Examples of suitable reporter genes include those which encode chloramphenicol acetyltransferase, beta-galactosidase, alkaline phosphatase, green fluorescent protein, or luciferase. Standard methods for measuring the activity of these gene products are known in the art.

A variety of cell types are suitable for use as an indicator cell in the screening assay. Preferably a cell line is used which expresses low levels of endogenous gene and is then engineered to express recombinant protein. Cells for use in the subject assays include eukaryotic cells. For example, in one embodiment, a cell is a fungal cell, such as a yeast cell. In another embodiment, a cell is a plant cell. In yet another embodiment, a cell is a vertebrate cell, e.g., an avian cell or a mammalian cell (e.g., a murine cell, or a human cell).

Recombinant expression vectors that can be used for expression of a target gene, e.g., LRRK2, AITC, AUF1, DCP1 and/or DPC2, are known in the art. For example, the cDNA is first introduced into a recombinant expression vector using standard molecular biology techniques. A cDNA can be obtained, for example, by amplification using the polymerase chain reaction (PCR) or by screening an appropriate cDNA library. The nucleotide sequences of cDNAs for or a molecule in a signal transduction pathway involving (e.g., human, murine and yeast) are known in the art and can be used for the design of PCR primers that allow for amplification of a cDNA by standard PCR methods or for the design of a hybridization probe that can be used to screen a cDNA library using standard hybridization methods.

In another embodiment, the test compounds can be subjected to a cell-free composition that includes the protein(s) (e.g., a cell extract or a composition that includes e.g., either purified natural or recombinant protein). LRRK2, AITC, AUF1, DCP1 and/or DPC2 expressed by recombinant methods in a host cells or culture medium can be isolated from the host cells, or cell culture medium using standard methods for protein purification. For example, ion-exchange chromatography, gel filtration chromatography, ultrafiltration, electrophoresis, and immunoaffinity purification with antibodies can be used to produce a purified or semi-purified protein that can be used in a cell free composition. Alternatively, a lysate or an extract of cells expressing the protein of interest can be prepared for use as cell-free composition.

In one embodiment, compounds that specifically modulate the activity of LRRK2, AITC, AUF1, DCP1 and/or DPC2, or the activity of a binding partner of LRRK2, AITC, AUF1, DCP1 and/or DPC2, are identified based on their ability to modulate the interaction of LRRK2, AITC, AUF1, DCP1 and/or DPC2 with their binding partners. The binding partner can be a mRNA molecule or a protein molecule. Suitable assays are known in the art that allow for the detection of protein-protein interactions (e.g., immunoprecipitations, two-hybrid assays and the like) or that allow for the detection of interactions between LRRK2, AITC, AUF1, DCP1 and/or DPC2 and an mRNA (e.g., electrophoretic mobility shift assays, DNAse I footprinting assays and the like). By performing such assays in the presence and absence of test compounds, these assays can be used to identify compounds that modulate (e.g., inhibit or enhance) the activity of LRRK2, AITC, AUF1, DCP1 and/or DPC2 with a binding ligand.

Compounds identified in the subject screening assays can be used in methods of modulating one or more of the biological responses regulated by LRRK2, AITC, AUF1, DCP1 and/or DPC2. It will be understood that it may be desirable to formulate such compound(s) as pharmaceutical compositions as described herein prior to contacting them with cells.

Once a test compound is identified that directly or indirectly modulates the expression or activity of LRRK2, AITC, AUF1, DCP1 and/or DPC2 by one of the variety of methods described hereinbefore, the selected test compound (or “compound of interest”) can then be further evaluated for its effect on cells, for example by contacting the compound of interest with cells either in vivo (e.g., by administering the compound of interest to an organism) or ex vivo (e.g., by isolating cells from an organism and contacting the isolated cells with the compound of interest or, alternatively, by contacting the compound of interest with a cell line) and determining the effect of the compound of interest on the cells, as compared to an appropriate control (such as untreated cells or cells treated with a control compound, or carrier, that does not modulate the biological response).

In another aspect, the invention pertains to a combination of two or more of the assays described herein. For example, a modulator can be identified using a cell-based or a cell-free assay, and the ability of the modulators to increase or decrease the activity of LRRK2, AITC, AUF1, DCP1 and/or DPC2, or a protein with which they interact, can be confirmed in vivo, e.g., in an animal, such as, for example, an animal model for, e.g., a tumor model.

Moreover, a modulator of LRRK2, AITC, AUF1, DCP1 and/or DPC2 or a molecule in a signaling pathway involving LRRK2, AITC, AUF1, DCP1 and/or DPC2 identified as described herein (e.g., an antisense nucleic acid molecule, or a specific antibody, or a small molecule) can be used in an animal model to determine the efficacy, toxicity, or side effects of treatment with such a modulator. Alternatively, a modulator identified as described herein can be used in an animal model to determine the mechanism of action of such a modulator.

In another embodiment, it will be understood that similar screening assays can be used to identify compounds that indirectly modulate the activity and/or expression of LRRK2, AITC, AUF1, DCP1 and/or DPC2, e.g., by performing screening assays such as those described above using molecules with which LRRK2, AITC, AUF1, DCP1 and/or DPC2 interacts, or any molecules that act either upstream or downstream of LRRK2, AITC, AUF1, DCP1 and/or DPC2 in the pathway.

Compounds identified by the screening assays of the present invention are considered as candidate therapeutic compounds useful for treating LRRK2-associated diseases, e.g., Parkinson's disease, as described herein. Thus, the invention also includes compounds identified in the screening assays, and methods for their administration and use in the treatment, prevention, or delay of development or progression of diseases described herein.

### III. Agents that Decrease the Expression of LRRK2

The present invention provides a novel regulatory mechanism of regulating LRRK2 through LRRK2 mRNA decay. Specifically, the inventors discovered that modulation of LRRK2 mRNA decay is a potential novel therapeutic strategy for treating or preventing LRRK2-associated disorder or condition, e.g., Parkinson's disease, which is distinct from targeting enzymatic functions of LRRK2.

Accordingly, in some embodiments, the present invention provide agents that decrease the expression of LRRK2, e.g., agents that induce LRRK2 mRNA decay. Examples of such agents that may be used in the methods and compositions described herein are provided below, and include, but are not limited to, small molecules, antibodies, or antigen-binding fragment thereof, peptides, proteins, fusion proteins, or nucleic acid molecules (e.g., antisense RNAs, dsRNAs, siRNAs, or an RNA-guided nuclease).

### Inhibiting Agents

In some embodiments, the agent that decreases the expression of LRRK2 is AICAR/ZMP, an enzymatic substrate of ATIC. In some embodiments, the agent that decreases the expression of LRRK2 is AICAr, a precursor of AICAR/ZMP. As demonstrated in the Examples, AICAr and AICAR/ZMP specifically regulate LRRK2 at mRNA level, e.g., via ARE-mediated mRNA decay. LRRK2 mRNA levels were significantly decreased upon AICAr treatment. In addition, AUF1 was found to interact with the AICAr response element of LRRK2 and plays a critical role in AICAr-induced LRRK2 suppression. AICAr treatment also significantly increased the level of the decapping enzymes DCP1/2, leading to removal of the 5′ cap of mRNA and therefore mRNA decay. Furthermore, AICAr treatment was able to rescue LRRK2-induced neurodegeneration and locomotor deficits in vivo.

AICAR/ZMP and/or AICAr levels may be measured directly or indirectly. AICAR/ZMP and/or AICAr levels in a cell may be quantitated directly, e.g., by extracting nucleotides from cells and separating the nucleotides by HPLC as described in (Sabina et al, J. Biol. Chem. 257(17) 10178-10183 (1982)). AICAR/ZMP and/or AICAr levels in a cell may be assessed indirectly, e.g., by monitoring AMPK activation, such as by quantifying phospho-AMPK levels in the cell using a commercially available antibody specific for phosphorylated AMPK.

In some embodiments, the agent that decreases the expression of LRRK2 is an inhibitor of 5-aminoimidazole-4-carboxamide ribonucleotide formyltransferase/IMP cyclohydrolase (ATIC). ATIC catalyzes the penultimate step in de novo purine biosynthesis. In some embodiments, the agent that decreases the expression of LRRK2 is 5-aminoimidazole-4-carboxamide riboside (AICAr), a precursor of AICAR/ZMP.

In some embodiments, the ATIC inhibitor is selected from the group consisting of a small molecule, an antagonist antibody of ATIC, or antigen-binding fragment thereof, an antisense agent targeting ATIC, a double stranded RNA agent targeting ATIC, an RNA-guided nuclease targeting ATIC, an ATIC fusion protein; and an ATIC inhibitory peptide, as described below.

### Inhibitory Nucleic Acids

In one embodiment, the methods described herein include targeting ATIC using inhibitory nucleic acids. A nucleic acid inhibitor can encode a small interference RNA (e.g., an RNAi agent) that targets the ATIC gene, or a gene encoding for another protein that interacts with ATIC, and inhibits the expression or activity. The term “RNAi agent” refers to an RNA, or analog thereof, having sufficient sequence complementarity to a target RNA to direct RNA interference. Examples also include a DNA that can be used to make the RNA.

RNA Interference: RNA interference (RNAi) refers to a sequence-specific or selective process by which a target molecule (e.g., a target gene, protein or RNA) is down-regulated. Generally, an interfering RNA (“RNAi”) is a double stranded short-interfering RNA (siRNA), short hairpin RNA (shRNA), or single-stranded micro-RNA (miRNA) that results in catalytic degradation of specific mRNAs, and also can be used to lower or inhibit gene expression. RNA interference (RNAi) is a process whereby double-stranded RNA (dsRNA) induces the sequence-specific regulation of gene expression in animal and plant cells and in bacteria (Aravin and Tuschl, FEBS Lett. 26:5830-5840 (2005); Herbert et al., Curr. Opin. Biotech. 19:500-505 (2008); Hutvagner and Zamore, Curr. Opin. Genet. Dev., 12: 225-232 (2002); Sharp, Genes Dev., 15:485-490 (2001); Valencia-Sanchez et al. Genes Dev. 20:515-524 (2006)). In mammalian cells, RNAi can be triggered by 21-nucleotide (nt) duplexes of small interfering RNA (siRNA) (Chiu et al., Mol. Cell. 10:549-561 (2002); Elbashir et al., Nature 411:494-498 (2001)), by microRNA (miRNA), functional small-hairpin RNA (shRNA), or other dsRNAs which are expressed in vivo using DNA templates with RNA polymerase II or III promoters (Zeng et al., Mol. Cell 9:1327-1333 (2002); Paddison et al., Genes Dev. 16:948-958 (2002); Denti, et al., Mol. Ther. 10:191-199 (2004); Lee et al., Nature Biotechnol. 20:500-505 (2002); Paul et al., Nature Biotechnol. 20:505-508 (2002); Rossi, Human Gene Ther. 19:313-317 (2008); Tuschl, T., Nature Biotechnol. 20:440-448 (2002); Yu et al., Proc. Natl. Acad. Sci. USA 99(9):6047-6052 (2002); McManus et al., RNA 8:842-850 (2002); Scherer et al., Nucleic Acids Res. 35:2620-2628 (2007); Sui et al., Proc. Natl. Acad. Sci. USA 99(6):5515-5520 (2002).)

siRNA Molecules: The term “short interfering RNA” or “siRNA” (also known as “small interfering RNAs”) refers to an RNA agent, preferably a double-stranded agent, of about 10-50 nucleotides in length, preferably between about 15-25 nucleotides in length, more preferably about 17, 18, 19, 20, 21, 22, 23, 24, or 25 nucleotides in length, the strands optionally having overhanging ends comprising, for example 1, 2 or 3 overhanging nucleotides (or nucleotide analogs), which is capable of directing or mediating RNA interference. Naturally-occurring siRNAs are generated from longer dsRNA molecules (e.g., >25 nucleotides in length) by a cell's RNAi machinery.

In general, the methods described herein can use dsRNA molecules comprising 16-30, e.g., 16, 17, 18, 19, 20, 21, 22, 23, 24, 25, 26, 27, 28, 29, or 30 nucleotides in each strand, wherein one of the strands is substantially identical, e.g., at least 80% (or more, e.g., 85%, 90%, 95%, or 100%) identical, e.g., having 3, 2, 1, or 0 mismatched nucleotide(s), to a target region in the mRNA, and the other strand is complementary to the first strand. The dsRNA molecules can be chemically synthesized, or can be transcribed in vitro or in vivo, e.g., shRNA, from a DNA template. The dsRNA molecules can be designed using any method known in the art. Negative control siRNAs should not have significant sequence complementarity to the appropriate genome. Such negative controls can be designed by randomly scrambling the nucleotide sequence of the selected siRNA; a homology search can be performed to ensure that the negative control lacks homology to any other gene in the appropriate genome. In addition, negative control siRNAs can be designed by introducing one or more base mismatches into the sequence.

The methods described herein can use both siRNA and modified siRNA derivatives, e.g., siRNAs modified to alter a property such as the specificity and/or pharmacokinetics of the composition, for example, to increase half-life in the body, e.g., crosslinked siRNAs. Thus, the invention includes methods of administering siRNA derivatives that include siRNA having two complementary strands of nucleic acid, such that the two strands are crosslinked. The oligonucleotide modifications include, but are not limited to, 2′-O-methyl, 2′-fluoro, 2′-O-methyoxyethyl and phosphorothioate, boranophosphate, 4′-thioribose. (Wilson and Keefe, Curr. Opin. Chem. Biol. 10:607-614 (2006); Prakash et al., J. Med. Chem. 48:4247-4253 (2005); Soutschek et al., Nature 432:173-178 (2004)).

In some embodiments, the siRNA derivative has at its 3′ terminus a biotin molecule (e.g., a photocleavable biotin), a peptide (e.g., a Tat peptide), a nanoparticle, a peptidomimetic, organic compounds (e.g., a dye such as a fluorescent dye), or dendrimer. Modifying siRNA derivatives in this way may improve cellular uptake or enhance cellular targeting activities of the resulting siRNA derivative as compared to the corresponding siRNA, are useful for tracing the siRNA derivative in the cell, or improve the stability of the siRNA derivative compared to the corresponding siRNA.

The inhibitory nucleic acid compositions can be unconjugated or can be conjugated to another moiety, such as a nanoparticle, to enhance a property of the compositions, e.g., a pharmacokinetic parameter such as absorption, efficacy, bioavailability, and/or half-life. The conjugation can be accomplished by methods known in the art, e.g., using the methods of Lambert et al., Drug Deliv. Rev.: 47(1), 99-112 (2001) (describes nucleic acids loaded to polyalkylcyanoacrylate (PACA) nanoparticles); Fattal et al., J. Control Release 53(1-3):137-43 (1998) (describes nucleic acids bound to nanoparticles); Schwab et al., Ann. Oncol. 5 Suppl. 4:55-8 (1994) (describes nucleic acids linked to intercalating agents, hydrophobic groups, polycations or PACA nanoparticles); and Godard et al., Eur. J. Biochem. 232(2):404-10 (1995) (describes nucleic acids linked to nanoparticles). The inhibitory nucleic acid molecules can also be labeled using any method known in the art; for instance, the nucleic acid compositions can be labeled with a fluorophore, e.g., Cy3, fluorescein, or rhodamine. The labeling can be carried out using a kit, e.g., the SILENCER™ siRNA labeling kit (Ambion). Additionally, the siRNA can be radiolabeled, e.g., using ³H, ³²P, or other appropriate isotope.

siRNA Delivery: Direct delivery of siRNA in saline or other excipients can silence target genes in tissues, such as the eye, lung, and central nervous system (Bitko et al., Nat. Med. 11:50-55 (2005); Shen et al., Gene Ther. 13:225-234 (2006); Thakker et al., Proc. Natl. Acad. Sci. U.S.A. (2004)). In adult mice, efficient delivery of siRNA can be accomplished by “high-pressure” delivery technique, a rapid injection (within 5 seconds) of a large volume of siRNA containing solution into animal via the tail vein (Liu (1999), supra; McCaffrey (2002), supra; Lewis, Nature Genetics 32:107-108 (2002)).

Liposomes and nanoparticles can also be used to deliver siRNA into animals. Delivery methods using liposomes, e.g. stable nucleic acid-lipid particles (SNALPs), dioleoyl phosphatidylcholine (DOPC)-based delivery system, as well as lipoplexes, e.g. Lipofectamine 2000, TransIT-TKO, have been shown to effectively repress target mRNA (de Fougerolles, Human Gene Ther. 19:125-132 (2008); Landen et al., Cancer Res. 65:6910-6918 (2005); Luo et al., Mol. Pain 1:29 (2005); Zimmermann et al., Nature 441:111-114 (2006)). Conjugating siRNA to peptides, RNA aptamers, antibodies, or polymers, e.g. dynamic polyconjugates, cyclodextrin-based nanoparticles, atelocollagen, and chitosan, can improve siRNA stability and/or uptake. (Howard et al., Mol. Ther. 14:476-484 (2006); Hu-Lieskovan et al., Cancer Res. 65:8984-8992 (2005); Kumar, et al., Nature 448:39-43; McNamara et al., Nat. Biotechnol. 24:1005-1015 (2007); Rozema et al., Proc. Natl. Acad. Sci. U.S.A. 104:12982-12987 (2007); Song et al., Nat. Biotechnol. 23:709-717 (2005); Soutschek (2004), supra; Wolfrum et al., Nat. Biotechnol. 25:1149-1157 (2007)).

Viral-mediated delivery mechanisms can also be used to induce specific silencing of targeted genes through expression of siRNA, for example, by generating recombinant adenoviruses harboring siRNA under RNA Pol II promoter transcription control (Xia et al. (2002), supra). Infection of HeLa cells by these recombinant adenoviruses allows for diminished endogenous target gene expression. Injection of the recombinant adenovirus vectors into transgenic mice expressing the target genes of the siRNA results in in vivo reduction of target gene expression. Id. In an animal model, whole-embryo electroporation can efficiently deliver synthetic siRNA into post-implantation mouse embryos (Calegari et al., Proc. Natl. Acad. Sci. USA 99(22):14236-40 (2002)).

Stable siRNA Expression: Synthetic siRNAs can be delivered into cells, e.g., by direct delivery, cationic liposome transfection, and electroporation. However, these exogenous siRNA typically only show short term persistence of the silencing effect (4-5 days). Several strategies for expressing siRNA duplexes within cells from recombinant DNA constructs allow longer-term target gene suppression in cells, including mammalian Pol II and III promoter systems (e.g., H1, Ul, or U6/snRNA promoter systems (Denti et al. (2004), supra; Tuschl (2002), supra); capable of expressing functional double-stranded siRNAs (Bagella et al., J. Cell. Physiol. 177:206-213 (1998); Lee et al. (2002), supra; Miyagishi et al. (2002), supra; Paul et al. (2002), supra; Scherer et al. (2007), supra; Yu et al. (2002), supra; Sui et al. (2002), supra).

Transcriptional termination by RNA Pol IIH occurs at runs of four consecutive T residues in the DNA template, providing a mechanism to end the siRNA transcript at a specific sequence. The siRNA is complementary to the sequence of the target gene in 5′-3′ and 3′-5′ orientations, and the two strands of the siRNA can be expressed in the same construct or in separate constructs. Hairpin siRNAs, driven by H1 or U6 snRNA promoter and expressed in cells, can inhibit target gene expression (Bagella et al. (1998), supra; Lee et al. (2002), supra; Miyagishi et al. (2002), supra; Paul et al. (2002), supra; Yu et al. (2002), supra; Sui et al. (2002) supra). Constructs containing siRNA sequence under the control of T7 promoter also make functional siRNAs when cotransfected into the cells with a vector expression T7 RNA polymerase (Jacque (2002), supra).

In another embodiment, siRNAs can be expressed in a miRNA backbone which can be transcribed by either RNA Pol II or III. MicroRNAs are endogenous noncoding RNAs of approximately 22 nucleotides in animals and plants that can post-transcriptionally regulate gene expression (Bartel, Cell 116:281-297 (2004); Valencia-Sanchez et al., Genes & Dev. 20:515-524 (2006)). One common feature of miRNAs is that they are excised from an approximately 70 nucleotide precursor RNA stem loop by Dicer, an RNase IIH enzyme, or a homolog thereof By substituting the stem sequences of the miRNA precursor with the sequence complementary to the target mRNA, a vector construct can be designed to produce siRNAs to initiate RNAi against specific mRNA targets in mammalian cells. When expressed by DNA vectors containing polymerase II or III promoters, miRNA designed hairpins can silence gene expression (McManus (2002), supra; Zeng (2002), supra).

Antisense: An “antisense” nucleic acid can include a nucleotide sequence that is complementary to a “sense” nucleic acid encoding a protein, e.g., complementary to the coding strand of a double-stranded cDNA molecule or complementary to a target mRNA sequence. The antisense nucleic acid can be complementary to an entire coding strand of a target sequence, or to only a portion thereof (for example, the coding region of a target gene). In another embodiment, the antisense nucleic acid molecule is antisense to a “noncoding region” of the coding strand of a nucleotide sequence encoding the selected target gene (e.g., the 5′ and 3′ untranslated regions).

An antisense nucleic acid can be designed such that it is complementary to the entire coding region of a target mRNA but can also be an oligonucleotide that is antisense to only a portion of the coding or noncoding region of the target mRNA. For example, the antisense oligonucleotide can be complementary to the region surrounding the translation start site of the target mRNA, e.g., between the −10 and +10 regions of the target gene nucleotide sequence of interest. An antisense oligonucleotide can be, for example, about 7, 10, 15, 20, 25, 30, 35, 40, 45, 50, 55, 60, 65, 70, 75, 80, or more nucleotides in length.

An antisense nucleic acid of the invention can be constructed using chemical synthesis and enzymatic ligation reactions using procedures known in the art. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids, e.g., phosphorothioate derivatives and acridine substituted nucleotides can be used. The antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

Based upon the sequences disclosed herein, e.g., sequences relating to ATIC, one of skill in the art can easily choose and synthesize any of a number of appropriate antisense molecules for use in accordance with the present invention. For example, a “gene walk” comprising a series of oligonucleotides of 15-30 nucleotides spanning the length of a target nucleic acid can be prepared, followed by testing for inhibition of target gene expression. Optionally, gaps of 5-10 nucleotides can be left between the oligonucleotides to reduce the number of oligonucleotides synthesized and tested.

The antisense nucleic acid molecules of the invention are typically administered to a subject (e.g., by direct injection at a tissue site), or generated in situ such that they hybridize with or bind to cellular mRNA and/or genomic DNA encoding a target protein to thereby inhibit expression of the protein, e.g., by inhibiting transcription and/or translation. Alternatively, antisense nucleic acid molecules can be modified to target selected cells and then administered systemically. For systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of the antisense molecules, vector constructs in which the antisense nucleic acid molecule is placed under the control of a strong pol II or pol III promoter can be used.

In some embodiments, the antisense nucleic acid is a morpholino oligonucleotide (see, e.g., Heasman, Dev. Biol. 243:209-14 (2002); Iversen, Curr. Opin. Mol. Ther. 3:235-8 (2001); Summerton, Biochim. Biophys. Acta. 1489:141-58 (1999).

Target gene expression can be inhibited by targeting nucleotide sequences complementary to a regulatory region, e.g., promoters and/or enhancers) to form triple helical structures that prevent transcription of the target gene in target cells. See generally, Helene, C. Anticancer Drug Des. 6:569-84 (1991); Helene, C. Ann. N.Y. Acad. Sci. 660:27-36 (1992); and Maher, Bioassays. 14:807-15 (1992). The potential sequences that can be targeted for triple helix formation can be increased by creating a so called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

### Fusion Proteins and RNA-Guided Nucleases

In another aspect of the invention, the agent that decreases the expression of LRRK2, e.g., an ATIC inhibitor, is a fusion protein.

As used herein, a “chimeric protein” or “fusion protein” comprises all or part (preferably a biologically active part) of a first protein operably linked to a heterologous second polypeptide (i.e., a polypeptide other than the first protein). Within the fusion protein, the term “operably linked” is intended to indicate that the first protein or segment thereof and the heterologous polypeptide are fused in-frame to each other. The heterologous polypeptide can be fused to the amino-terminus or the carboxyl-terminus of the first protein or segment.

In some embodiments, the fusion proteins of the invention include ATIC fused to an effector molecule. In some embodiments, the fusion proteins of the invention include a protein that interacts with ATIC fused to an effector molecule. Exemplary effector molecules include include, for example, nucleases, physical blockers, epigenetic recruiters, e.g., a transcriptional repressor, and epigenetic CpG modifiers, e.g., a DNA methylase, a DNA demethylase, a histone modifying agent, or a histone deacetylase, and combinations of any of the foregoing.

In one embodiment, the agents used to inhibit ATIC expression and/or activity are based on CRISPR technology and are RNA-guided nucleases targeting ATIC, or any other protein that interacts with ATIC.

The clustered, regularly interspaced, short palindromic repeat (CRISPR) technology is included in the invention as an approach for generating RNA-guided nuclease with customizable specificities for targeted genome editing. Genome editing mediated by these nucleases has been used to rapidly, easily and efficiently modify endogenous genes in a wide variety of biomedically important cell types and in organisms that have traditionally been challenging to manipulate genetically.

In general, the term “CRISPR system” refers collectively to transcripts and other/elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In embodiments of the invention the terms guide sequence and guide RNA are used interchangeably. In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise any polynucleotide, such as DNA or RNA polynucleotides (e.g., DNA or RNA of ATIC). In some embodiments, a target sequence is located in the nucleus or cytoplasm of a cell.

In preferred embodiments of the invention, the CRISPR/Cas system is a type II CRISPR system and the Cas enzyme is Cas9, which catalyzes DNA cleavage. Enzymatic action by Cas9 derived from Streptococcus pyogenes or any closely related Cas9 generates double stranded breaks at target site sequences which hybridize to 20 nucleotides of the guide sequence and that have a protospacer-adjacent motif (PAM) sequence NGG following the 20 nucleotides of the target sequence. CRISPR activity through Cas9 for site-specific DNA recognition and cleavage is defined by the guide sequence, the tracr sequence that hybridizes in part to the guide sequence and the PAM sequence. More aspects of the CRISPR system are described in Karginov and Hannon, The CRISPR system: small RNA-guided defense in bacteria and archae, Mol. Cell 2010, January 15; 37(1): 7.

The type II CRISPR locus from Streptococcus pyogenes SF370, which contains a cluster of four genes Cas9, Cas1, Cas2, and Csn1, as well as two non-coding RNA elements, tracrRNA and a characteristic array of repetitive sequences (direct repeats) interspaced by short stretches of non-repetitive sequences (spacers, about 30 bp each). In this system, targeted DNA double-strand break (DSB) is generated in four sequential steps. First, two non-coding RNAs, the pre-crRNA array and tracrRNA, are transcribed from the CRISPR locus. Second, tracrRNA hybridizes to the direct repeats of pre-crRNA, which is then processed into mature crRNAs containing individual spacer sequences. Third, the mature crRNA:tracrRNA complex directs Cas9 to the DNA target consisting of the protospacer and the corresponding PAM via heteroduplex formation between the spacer region of the crRNA and the protospacer DNA. Finally, Cas9 mediates cleavage of target DNA upstream of PAM to create a DSB within the protospacer. Several aspects of the CRISPR system can be further improved to increase the efficiency and versatility of CRISPR targeting. Optimal Cas9 activity may depend on the availability of free Mg2+ at levels higher than that present in the mammalian nucleus (see e.g. Jinek et al., 2012, Science, 337:816), and the preference for an NGG motif immediately downstream of the protospacer restricts the ability to target on average every 12-bp in the human genome.

Typically, in the context of an endogenous CRISPR system, formation of a CRISPR complex (comprising a guide sequence hybridized to a target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. Without wishing to be bound by theory, the tracr sequence, which may comprise or consist of all or a portion of a wild-type tracr sequence (e.g., about or more than about 20, 26, 32, 45, 48, 54, 63, 67, 85, or more nucleotides of a wild-type tracr sequence), may also form part of a CRISPR complex, such as by hybridization along at least a portion of the tracr sequence to all or a portion of a tracr mate sequence that is operably linked to the guide sequence. In some embodiments, one or more vectors driving expression of one or more elements of a CRISPR system are introduced into a host cell such that expression of the elements of the CRISPR system direct formation of a CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter drives expression of a transcript encoding a CRISPR enzyme and one or more of the guide sequence, tracr mate sequence (optionally operably linked to the guide sequence), and a tracr sequence embedded within one or more intron sequences (e.g. each in a different intron, two or more in at least one intron, or all in a single intron). In some embodiments, the CRISPR enzyme, guide sequence, tracr mate sequence, and tracr sequence are operably linked to and expressed from the same promoter.

The expression of a target polynucleotide can be modified by allowing a CRISPR complex to bind to the polynucleotide, wherein the CRISPR complex comprises a CRISPR enzyme complexed with a guide sequence hybridized to a target sequence within said polynucleotide, e.g., an RNA-guided nuclease targeting ATIC, wherein said guide sequence is linked to a tracr mate sequence which in turn hybridizes to a tracr sequence. In some embodiment, binding of CRISPR complex to a target polynucleotide results in an increased expression of the target polynucleotide. In another embodiment, binding of CRISPR complex to a target polynucleotide results in a decreased expression of the target polynucleotide (e.g., DNA or RNA of ATIC).

In some embodiment, the fusion protein may comprise an effector, such as a nuclease, e.g., a Cas9, e.g., a wild type Cas9, a nickase Cas9 (e.g., Cas9 D10A), a dead Cas9 (dCas9), eSpCas9, Cpf1, C2C1, or C2C3, or a nucleic acid encoding such a nuclease. The choice of nuclease and gRNA(s) is determined by whether the targeted mutation is a deletion, substitution, or addition of nucleotides, e.g., a deletion, substitution, or addition of nucleotides to a targeted sequence. Fusions of a catalytically inactive endonuclease e.g., a dead Cas9 (dCas9, e.g., D10A; H840A) tethered with all or a portion of (e.g., biologically active portion of) an (one or more) effector domain create chimeric proteins that can be linked to the polypeptide to guide the composition to specific DNA sites by one or more RNA sequences (e.g., DNA recognition elements including, but not restricted to zinc finger arrays, sgRNA, TAL arrays, peptide nucleic acids described herein) to modulate activity and/or expression of one or more target nucleic acids sequences (e.g., to methylate or demethylate a DNA sequence).

In one embodiment, a fusion protein of the invention may comprise an effector molecule comprising, for example, a CRISPER associated protein (Cas) polypeptide, or fragment thereof, (e.g., a Cas9 polypeptide, or fragment thereof) and an epigenetic recruiter or an epigenetic CpG modifier.

In one embodiment, a suitable Cas polypeptide is an enzymatically inactive Cas polypeptide, e.g., a “dead Cas polypeptide” or “dCas” polypeptide

Exemplary Cas polypeptides that are adaptable to the methods and compositions described herein are described below. Using methods known in the art, a Cas polypeptide can be fused to any of a variety of agents and/or molecules as described herein; such resulting fusion molecules can be useful in various disclosed methods.

In one aspect, the invention includes a composition comprising a protein comprising a domain, e.g., an effector, that acts on DNA (e.g., a nuclease domain, e.g., a Cas9 domain, e.g., a dCas9 domain; a DNA methyltransferase, a demethylase, a deaminase), in combination with at least one guide RNA (gRNA) or antisense DNA oligonucleotide that targets the protein to site-specific target sequence, wherein the composition is effective to alter, in a human cell, the expression of a target gene. In some embodiments, the enzyme domain is a Cas9 or a dCas9. In some embodiments, the protein comprises two enzyme domains, e.g., a dCas9 and a methylase or demethylase domain.

In one aspect, the invention includes a composition comprising a protein comprising a domain, e.g., an effector, that comprises a transcriptional control element (e.g., a nuclease domain, e.g., a Cas9 domain, e.g., a dCas9 domain; a transcriptional enhancer, a transcriptional repressor), in combination with at least one guide RNA (gRNA) or antisense DNA oligonucleotide that targets the protein to a site-specific target sequence, wherein the composition is effective to alter, in a human cell, the expression of a target gene. In some embodiments, the enzyme domain is a Cas9 or a dCas9. In some embodiments, the protein comprises two enzyme domains, e.g., a dCas9 and a transcriptional enhancer or transcriptional repressor domain.

As used herein, a “biologically active portion of an effector domain” is a portion that maintains the function (e.g., completely, partially, minimally) of an effector domain (e.g., a “minimal” or “core” domain).

The chimeric proteins described herein may also comprise a linker, e.g., an amino acid linker. In some aspects, a linker comprises 2 or more amino acids, e.g., one or more GS sequences. In some aspects, fusion of Cas9 (e.g., dCas9) with two or more effector domains (e.g., of a DNA methylase or enzyme with a role in DNA demethylation or protein acetyl transferase or deacetylase) comprises one or more interspersed linkers (e.g., GS linkers) between the domains. In some aspects, dCas9 is fused with 2-5 effector domains with interspersed linkers.

A variety of CRISPR associated (Cas) genes or proteins can be used in the present invention and the choice of Cas protein will depend upon the particular conditions of the method.

Specific examples of Cas proteins include class II systems including Cas1, Cas2, Cas3, Cas4, Cas5, Cas6, Cas7, Cas8, Cas9, Cas1O, Cpf1, C2C1, or C2C3. In some embodiments, a Cas protein, e.g., a Cas9 protein, may be from any of a variety of prokaryotic species. In some embodiments a particular Cas protein, e.g., a particular Cas9 protein, is selected to recognize a particular protospacer-adjacent motif (PAM) sequence. In some embodiments, the site-specific targeting moiety includes a sequence targeting polypeptide, such as an enzyme, e.g., Cas9. In certain embodiments a Cas protein, e.g., a Cas9 protein, may be obtained from a bacteria or archaea or synthesized using known methods. In certain embodiments, a Cas protein may be from a gram positive bacteria or a gram negative bacteria. In certain embodiments, a Cas protein may be from a Streptococcus, (e.g., a S. pyogenes, a S. thermophilus) a Cryptococcus, a Corynebacterium, a Haemophilus, a Eubacterium, a Pasteurella, a Prevotella, a Veillonella, or a Marinobacter. In some embodiments nucleic acids encoding two or more different Cas proteins, or two or more Cas proteins, may be introduced into a cell, zygote, embryo, or animal, e.g., to allow for recognition and modification of sites comprising the same, similar or different PAM motifs. In some embodiments, the Cas protein is modified to deactivate the nuclease, e.g., nuclease-deficient Cas9, and to recruit transcription activators or repressors, e.g., the co-subunit of the E. coli Pol, VP64, the activation domain of p65, KRAB, or SID4X, to induce epigenetic modifications, e.g., histone acetyltransferase, histone methyltransferase and demethylase, DNA methyltransferase and enzyme with a role in DNA demethylation (e.g., the TET family enzymes catalyze oxidation of 5-methylcytosine to 5-hydroxymethylcytosine and higher oxidative derivatives).

For the purposes of gene editing, CRISPR arrays can be designed to contain one or multiple guide RNA sequences corresponding to a desired target DNA sequence; see, for example, Cong et al. (2013) Science, 339:819-823; Ran et al. (2013) Nature Protocols, 8:2281-2308. At least about 16 or 17 nucleotides of gRNA sequence are required by Cas9 for DNA cleavage to occur, for Cpf1 at least about 16 nucleotides of gRNA sequence is needed to achieve detectable DNA cleavage.

Whereas wild-type Cas9 generates double-strand breaks (DSBs) at specific DNA sequences targeted by a gRNA, a number of CRISPR endonucleases having modified functionalities are available, for example: a “nickase” version of Cas9 generates only a single-strand break; a catalytically inactive Cas9 (“dCas9”) does not cut the target DNA but interferes with transcription by steric hindrance. dCas9 can further be fused with a heterologous effector to repress (CRISPRi) or activate (CRISPRa) expression of a target gene. For example, Cas9 can be fused to a transcriptional silencer (e.g., a KRAB domain) or a transcriptional activator (e.g., a dCas9-VP64 fusion). A catalytically inactive Cas9 (dCas9) fused to Fok1 nuclease (“dCas9-FokI”) can be used to generate DSBs at target sequences homologous to two gRNAs. See, e. g., the numerous CRISPR/Cas9 plasmids disclosed in and publicly available from the Addgene repository (Addgene, 75 Sidney St., Suite 550A, Cambridge, MA 02139; addgene.org/crispr). A “double nickase” Cas9 that introduces two separate double-strand breaks, each directed by a separate guide RNA, is described as achieving more accurate genome editing by Ran et al. (2013) Cell, 154: 1380-1389.

CRISPR technology for editing the genes of eukaryotes is disclosed in US Patent Application Publications 2016/0138008A1 and US2015/0344912A1, and in U.S. Pat. Nos. 8,697,359, 8,771,945, 8,945,839, 8,999,641, 8,993,233, 8,895,308, 8,865,406, 8,889,418, 8,871,445, 8,889,356, 8,932,814, 8,795,965, and 8,906,616. Cpf1 endonuclease and corresponding guide RNAs and PAM sites are disclosed in US Patent Application Publication 2016/0208243 A1.

In some embodiments, an effector comprises one or more components of a CRISPR system described hereinabove.

In some embodiments, suitable effectors for use in the agents, compositions, and methods of the invention include, for example, nucleases, physical blockers, epigenetic recruiters, e.g., a transcriptional enhancer or a transcriptional repressor, and epigenetic CpG modifiers, e.g., a DNA methylase, a DNA demethylase, a histone modifying agent, or a histone deacetylase, and combinations of any of the foregoing.

Exemplary effectors include ubiquitin, bicyclic peptides as ubiquitin ligase inhibitors, transcription factors, DNA and protein modification enzymes such as topoisomerases, topoisomerase inhibitors such as topotecan, DNA methyltransferases such as the DNMT family (e.g., DNMT3a, DNMT3b, DNMTL), protein methyltransferases (e.g., viral lysine methyltransferase (vSET), protein-lysine N-methyltransferase (SMYD2), deaminases (e.g., APOBEC, UG1), histone methyltransferases such as enhancer of zeste homolog 2 (EZH2), PRMT1, histone-lysine-N-methyltransferase (Setdbl), histone methyltransferase (SET2), euchromatic histone-lysine N-methyltransferase 2 (G9a), histone-lysine N-methyltransferase (SUV39H1), and G9a), histone deacetylase (e.g., HDAC1, HDAC2, HDAC3), enzymes with a role in DNA demethylation (e.g., the TET family enzymes catalyze oxidation of 5-methylcytosine to 5-hydroxymethylcytosine and higher oxidative derivatives), protein demethylases such as KDMIA and lysine-specific histone demethylase 1 (LSD1), helicases such as DHX9, acetyltransferases, deacetylases (e.g., sirtuin 1, 2, 3, 4, 5, 6, or 7), kinases, phosphatases, DNA-intercalating agents such as ethidium bromide, sybr green, and proflavine, efflux pump inhibitors such as peptidomimetics like phenylalanine arginyl-naphthylamide or quinoline derivatives, nuclear receptor activators and inhibitors, proteasome inhibitors, competitive inhibitors for enzymes such as those involved in lysosomal storage diseases, zinc finger proteins, TALENs, specific domains from proteins, such as a KRAB domain, a VP64 domain, a p300 domain (e.g., p300 core domain), an MeCP2 domain, an MQ1 domain, a DNMT3a-3L domain a TET1 domain, and a TET2 domain, protein synthesis inhibitors, nucleases (e.g., Cpf1, Cas9, zinc finger nuclease), fusions of one or more thereof (e.g., dCas9-DNMT, dCas9-APOBEC, dCas9-UG1, dCas9-VP64, dCas9-p300 core, dCas9-KRAB, dCas9-KRAB-MeCP2, dCas9-MQ1, dCas9-DNMT3a-3L, dCAS9-TET1, dCAS9-TET2, and dCas9-MC/MN).

In some embodiments, a suitable nuclease for use in the agent, compositions, and methods of the invention comprises a transcription activator like effector nucleases (TALEN). In yet other embodiments, a suitable nuclease comprises a zinc finger protein.

The term TALEN, as used herein, is broad and includes a monomeric TALEN that can cleave double stranded DNA without assistance from another TALEN. The term TALEN is also used to refer to one or both members of a pair of TALENs that are engineered to work together to cleave DNA at the same site. TALENs that work together may be referred to as a left-TALEN and a right-TALEN, which references the handedness of DNA. See U.S. Ser. No. 12/965,590; U.S. Ser. No. 13/426,991 (U.S. Pat. No. 8,450,471); U.S. Ser. No. 13/427,040 (U.S. Pat. No. 8,440,431); U.S. Ser. No. 13/427,137 (U.S. Pat. No. 8,440,432); and U.S. Ser. No. 13/738,381, all of which are incorporated by reference herein in their entirety.

TAL effectors are proteins secreted by Xanthomonas bacteria. The DNA binding domain contains a highly conserved 33-34 amino acid sequence with the exception of the 12th and 13th amino acids. These two locations are highly variable (Repeat Variable Diresidue (RVD)) and show a strong correlation with specific nucleotide recognition. This simple relationship between amino acid sequence and DNA recognition has allowed for the engineering of specific DNA binding domains by selecting a combination of repeat segments containing the appropriate RVDs.

The non-specific DNA cleavage domain from the end of the FokI endonuclease can be used to construct hybrid nucleases that are active in a yeast assay. These reagents are also active in plant cells and in animal cells. Initial TALEN studies used the wild-type FokI cleavage domain, but some subsequent TALEN studies also used FokI cleavage domain variants with mutations designed to improve cleavage specificity and cleavage activity. The FokI domain functions as a dimer, requiring two constructs with unique DNA binding domains for sites in the target genome with proper orientation and spacing. Both the number of amino acid residues between the TALEN DNA binding domain and the FokI cleavage domain and the number of bases between the two individual TALEN binding sites are parameters for achieving high levels of activity. The number of amino acid residues between the TALEN DNA binding domain and the FokI cleavage domain may be modified by introduction of a spacer (distinct from the spacer sequence) between the plurality of TAL effector repeat sequences and the FokI endonuclease domain. The spacer sequence may be 12 to 30 nucleotides.

The relationship between amino acid sequence and DNA recognition of the TALEN binding domain allows for designable proteins. In this case artificial gene synthesis is problematic because of improper annealing of the repetitive sequence found in the TALE binding domain. One solution to this is to use a publicly available software program (DNAWorks) to calculate oligonucleotides suitable for assembly in a two step PCR; oligonucleotide assembly followed by whole gene amplification. A number of modular assembly schemes for generating engineered TALE constructs have also been reported. Both methods offer a systematic approach to engineering DNA binding domains that is conceptually similar to the modular assembly method for generating zinc finger DNA recognition domains.

Once the TALEN genes have been assembled, they are inserted into plasmids; the plasmids are then used to transfect the target cell where the gene products are expressed and enter the nucleus to access the genome. TALENs can be used to edit genomes by inducing double-strand breaks (DSB), which cells respond to with repair mechanisms. In this manner, they can be used to correct mutations in the genome which, for example, cause disease.

As used herein, a “zinc finger polypeptide” or “zinc finger protein” is a protein that binds to DNA, RNA and/or protein, in a sequence-specific manner, by virtue of a metal stabilized domain known as a zinc finger. Zinc finger proteins are nucleases having a DNA cleavage domain and a DNA binding zinc finger domain. Zinc finger polypeptides may be made by fusing the nonspecific DNA. cleavage domain of an endonuclease with site-specific DNA binding zinc finger domains. Such nucleases are powerful tools for gene editing and can be assembled to induce double strand breaks (DSBs) site-specifically into genomic DNA. ZFNs allow specific gene disruption as during DNA repair, the targeted genes can be disrupted via mutagenic non-homologous end joint (NHEJ) or modified via homologous recombination (HR) if a closely related DNA template is supplied.

Zinc finger nucleases are chimeric enzymes made by fusing the nonspecific DNA. cleavage domain of the endonuclease FokI with site-specific DNA binding zinc finger domains. Due to the flexible nature of zinc finger proteins (ZFPs), ZFNs can be assembled that induce double strand breaks (DSBs) site-specifically into genomic DNA. ZFNs allow specific gene disruption as during DNA repair, the targeted genes can be disrupted via mutagenic non-homologous end joint (NHEJ) or modified via homologous recombination (HR) if a closely related DNA template is supplied.

In some embodiments, a suitable physical blocker for use in the agent, compositions, and methods of the invention comprises a gRNA, antisense DNA, or triplex forming oligonucleotide (which may target an expression control unit) steric block a transcriptional control element or anchoring sequence. The gRNA recognizes specific DNA sequences and further includes sequences that interfere with, e.g., a conjunction nucleating molecule sequence to act as a steric blocker. In some embodiments, the gRNA is combined with one or more peptides, e.g., S-adenosyl methionine (SAM), that acts as a steric presence. In other embodiments, a physical blocker comprises an enzymatically inactive Cas9 polypeptide, or fragment thereof (e.g., dCas9).

In one embodiment, an epigenetic recruiter activates or enhances transcription of a target gene, e.g., a gene that inhibits the expression and/or activity of ATIC.

In one embodiment, an epigenetic recruiter silences or represses transcription of a target gene, e.g., a gene that activates the expression and/or activity of ATIC.

In one embodiment, an epigenetic CpG modifier methylates DNA and inactivates or represses transcription.

In one embodiment, an epigenetic CpG modifier demethylates DNA and activates or stimulates transcription.

### Antibodies or Antigen Binding Fragments Thereof

The agents used in the methods of the present invention further contemplate anti-ATIC antibodies or antigen binding fragments thereof, thereby decreasing the expression and/or activity of ATIC in a cell, and decreasing the LRRK2 expression in the cell.

In another aspect, the invention also contemplates methods and compositions comprising an antibody which binds to a protein that interacts with ATIC, thereby inhibiting the expression and/or activity of the interacting protein, in a cell.

The term “antibody,” as used herein, is a broad term and is used in its ordinary sense, including, without limitation, to refer to naturally occurring antibodies as well as non-naturally occurring antibodies, including, for example, single chain antibodies, chimeric, bifunctional and humanized antibodies, as well as antigen-binding fragments thereof. An “antibody” refers to a glycoprotein comprising at least two heavy (H) chains and two light (L) chains inter-connected by disulfide bonds, or an antigen-binding portion thereof. Each heavy chain is comprised of a heavy chain variable region (abbreviated herein as V_{H}) and a heavy chain constant region. The heavy chain constant region is comprised of three domains, C_{H1}, C_{H2} and C_{H3}. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from N terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

The term “antigen-binding portion” of an antibody (or simply “antibody portion”), as used herein, refers to one or more fragments of an antibody that retain the ability to specifically bind to an antigen (e.g., ATIC). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fab′ fragment, which is essentially an Fab with part of the hinge region (see, FUNDAMENTAL IMMUNOLOGY (Paul ed., 3rd ed. 1993); (iv) a Fd fragment consisting of the VH and CH1 domains; (v) a Fv fragment consisting of the VL and VH domains of a single arm of an antibody, (vi) a dAb fragment (Ward et al. (1989) Nature 341: 544-546), which consists of a VH domain; (vii) an isolated complementarity determining region (CDR); and (viii) a nanobody, a heavy chain variable region containing a single variable domain and two constant domains. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242: 423-426; and Huston et al. (1988) Proc. Natl. Acad. Sci. USA 85: 5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

An antibody (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. An antibody includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

An “isolated antibody”, as used herein, is intended to refer to an antibody that is substantially free of other antibodies having different antigenic specificities (e.g., an isolated antibody that specifically binds to, e.g., ATIC, is substantially free of antibodies that specifically bind antigens other than ATIC). Moreover, an isolated antibody may be substantially free of other cellular material and/or chemicals. An “isolated antibody” may, however, include polyclonal antibodies, which all bind specifically to, e.g., ATIC.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human antibody”, as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The term “human monoclonal antibody” refers to antibodies displaying a single binding specificity, which have variable regions in which both the framework and CDR regions are derived from human germline immunoglobulin sequences. In one embodiment, the human monoclonal antibodies are produced by a hybridoma, which includes a B cell obtained from a transgenic nonhuman animal, e.g., a transgenic mouse, having a genome comprising a human heavy chain transgene and a light chain transgene fused to an immortalized cell.

The term “recombinant human antibody”, as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

As used herein, “isotype” refers to the antibody class (e.g., IgM or IgG1) that is encoded by the heavy chain constant region genes.

The phrases “an antibody recognizing an antigen” and “an antibody specific for an antigen” are used interchangeably herein with the term “an antibody which binds specifically to an antigen.”

The term “human antibody derivatives” refers to any modified form of the human antibody, e.g., a conjugate of the antibody and another agent or antibody.

The term “humanized antibody” is intended to refer to antibodies in which CDR sequences derived from the germline of another mammalian species, such as a mouse, have been grafted onto human framework sequences. Additional framework region modifications may be made within the human framework sequences. It will be appreciated by one of skill in the art that when a sequence is “derived” from a particular species, said sequence may be a protein sequence, such as when variable region amino acids are taken from a murine antibody, or said sequence may be a DNA sequence, such as when variable region encoding nucleic acids are taken from murine DNA. A humanized antibody may also be designed based on the known sequences of human and non-human (e.g., murine or rabbit) antibodies. The designed antibodies, potentially incorporating both human and non-human residues, may be chemically synthesized. The sequences may also be synthesized at the DNA level and expressed in vitro or in vivo to generate the humanized antibodies.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

The term “antibody mimetic” or “antibody mimic” is intended to refer to molecules capable of mimicking an antibody's ability to bind an antigen, but which are not limited to native antibody structures. Examples of such antibody mimetics include, but are not limited to, Adnectins (i.e., fibronectin based binding molecules), Affibodies, DARPins, Anticalins, Avimers, and Versabodies all of which employ binding structures that, while they mimic traditional antibody binding, are generated from and function via distinct mechanisms. The embodiments of the instant invention, as they are directed to antibodies, or antigen-binding portions thereof, also apply to the antibody mimetics described above.

As used herein, an antibody that “specifically binds” to an antigen, e.g., ATIC, is intended to refer to an antibody that binds to the antigen with a K_{D} of 1×10⁻⁷ M or less, more preferably 5×10⁻⁸ M or less, more preferably 1×10⁻⁸ M or less, more preferably 5×10⁻⁹ M or less.

The term “does not substantially bind” to a protein or cells, as used herein, means does not bind or does not bind with a high affinity to the protein or cells, i.e., binds to the protein or cells with a K_{D} of 1×10⁻⁶ M or more, more preferably 1×10⁻⁵ M or more, more preferably 1×10⁻⁴ M or more, more preferably 1×10⁻³ M or more, even more preferably 1×10⁻² M or more.

The term “K_{assoc}” or “K₈”, as used herein, is intended to refer to the association rate of a particular antibody-antigen interaction, whereas the term “K_{dis}” or “K_{d},” as used herein, is intended to refer to the dissociation rate of a particular antibody-antigen interaction. The term “K_{D}”, as used herein, is intended to refer to the dissociation constant, which is obtained from the ratio of K_{d} to Kₐ(i.e., K_{d}/Kₐ) and is expressed as a molar concentration (M). K_{D} values for antibodies can be determined using methods well established in the art. A preferred method for determining the K_{D} of an antibody is by using surface plasmon resonance, preferably using a biosensor system such as a Biacore® system.

As used herein, the term “high affinity”, when referring an IgG type antibody, refers to an antibody having a K_{D} of 10⁻⁸ M or less, more preferably 10⁻⁹ M or less and even more preferably 10⁻¹⁰ M or less for, e.g., ATIC. However, “high affinity” binding can vary for other antibody isotypes. For example, “high affinity” binding for an IgM isotype refers to an antibody having a K_{D} of 10⁻⁷ M or less, more preferably 10⁻⁸ M or less, even more preferably 10⁻⁹ M or less.

Preferrably, the antibody binds to ATIC with a K_{D} of 5×10⁻⁸ M or less, a K_{D} of 1×10⁻⁸ M or less, a K_{D} of 5×10⁻⁹ M or less, or a K_{D} of between 1×10⁻⁸ M and 1×10⁻¹⁰ M or less. Standard assays to evaluate the binding ability of the antibodies toward ATIC are known in the art, including for example, ELISAs, Western blots and RIAs. The binding kinetics (e.g., binding affinity) of the antibodies also can be assessed by standard assays known in the art, such as by ELISA, Scatchard and Biacore analysis.

### Engineered and Modified Antibodies

The V_{H} and/or V_{L} sequences of an antibody prepared according the methods of the present invention and may be used as starting material to engineer a modified antibody, which modified antibody may have altered properties from the starting antibody. An antibody can be engineered by modifying one or more residues within one or both of the original variable regions (i.e., V_{H} and/or V_{L}), for example within one or more CDR regions and/or within one or more framework regions. Additionally or alternatively, an antibody can be engineered by modifying residues within the constant region(s), for example to alter the effector function(s) of the antibody.

One type of variable region engineering that can be performed is CDR grafting. Antibodies interact with target antigens predominantly through amino acid residues that are located in the six heavy and light chain complementarity determining regions (CDRs). For this reason, the amino acid sequences within CDRs are more diverse between individual antibodies than sequences outside of CDRs. Because CDR sequences are responsible for most antibody-antigen interactions, it is possible to express recombinant antibodies that mimic the properties of specific naturally occurring antibodies by constructing expression vectors that include CDR sequences from the specific naturally occurring antibody grafted onto framework sequences from a different antibody with different properties (see, e.g., Riechmann et al. (1998) Nature 332: 323-327; Jones et al. (1986) Nature 321: 522-525; Queen et al. (1989) Proc. Nal. Acad. Sci. U.S.A. 86: 10029-10033; U.S. Pat. No. 5,225,539 to Winter, and U.S. Pat. Nos. 5,530,101; 5,585,089; 5,693,762 and 6,180,370 to Queen et al.)

Framework sequences for antibodies can be obtained from public DNA databases or published references that include germline antibody gene sequences. For example, germline DNA sequences for human heavy and light chain variable region genes can be found in the “VBase” human germline sequence database (available on the Internet at mrc-cpe.cam.ac.uk/vbase), as well as in Kabat, E. A., et al. (1991) Sequences of Proteins of Immunological Interest, Fifth Edition, U.S. Department of Health and Human Services, NIH Publication No. 91-3242; Tomlinson et al. (1992) J. Mol. Biol. 227: 776-798; and Cox et al. (1994) Eur. J. Immunol. 24: 827-836; the contents of each of which are expressly incorporated herein by reference. As another example, the germline DNA sequences for human heavy and light chain variable region genes can be found in the Genbank database.

Antibody protein sequences are compared against a compiled protein sequence database using one of the sequence similarity searching methods called the Gapped BLAST (Altschul et al. (1997) Nucleic Acids Res. 25: 3389-3402), which is well known to those skilled in the art. BLAST is a heuristic algorithm in that a statistically significant alignment between the antibody sequence and the database sequence is likely to contain high-scoring segment pairs (HSP) of aligned words. Segment pairs whose scores cannot be improved by extension or trimming is called a hit. Briefly, the nucleotide sequences of VBASE origin (vbase.mrc-cpe.cam.ac.uk/vbase1/list2.php) are translated and the region between and including FR1 through FR3 framework region is retained. The database sequences have an average length of 98 residues. Duplicate sequences, which are exact matches over the entire length of the protein, are removed. A BLAST search for proteins using the program blastp with default, standard parameters except the low complexity filter, which is turned off, and the substitution matrix of BLOSUM62, filters for the top 5 hits yielding sequence matches. The nucleotide sequences are translated in all six frames and the frame with no stop codons in the matching segment of the database sequence is considered the potential hit. This is in turn confirmed using the BLAST program tblastx, which translates the antibody sequence in all six frames and compares those translations to the VBASE nucleotide sequences dynamically translated in all six frames. Other human germline sequence databases, such as that available from IMGT (http://imgt.cines.fr), can be searched similarly to VBASE as described above.

The identities are exact amino acid matches between the antibody sequence and the protein database over the entire length of the sequence. The positives (identities+substitution match) are not identical but amino acid substitutions guided by the BLOSUM62 substitution matrix. If the antibody sequence matches two of the database sequences with same identity, the hit with most positives would be decided to be the matching sequence hit.

Identified V_{H} CDR1, CDR2, and CDR3 sequences, and the V_{K} CDR1, CDR2, and CDR3 sequences, can be grafted onto framework regions that have the identical sequence as that found in the germline immunoglobulin gene from which the framework sequence derives, or the CDR sequences can be grafted onto framework regions that contain one or more mutations as compared to the germline sequences. For example, it has been found that in certain instances it is beneficial to mutate residues within the framework regions to maintain or enhance the antigen-binding ability of the antibody (see e.g., U.S. Pat. Nos. 5,530,101; 5,585,089; 5,693,762 and 6,180,370 to Queen et al).

Another type of variable region modification is to mutate amino acid residues within the V_{H} and/or V_{K} CDR1, CDR2 and/or CDR3 regions to thereby improve one or more binding properties (e.g., affinity) of the antibody of interest. Site-directed mutagenesis or PCR-mediated mutagenesis can be performed to introduce the mutation(s) and the effect on antibody binding, or other functional property of interest, can be evaluated in in vitro or in vivo assays known in the art. For example, an antibody of the present invention may be mutated to create a library, which may then be screened for binding to an antigen, e.g., ATIC. Preferably conservative modifications (as discussed above) are introduced. The mutations may be amino acid substitutions, additions or deletions, but are preferably substitutions. Moreover, typically no more than one, two, three, four or five residues within a CDR region are altered.

Another type of framework modification involves mutating one or more residues within the framework region, or even within one or more CDR regions, to remove T cell epitopes to thereby reduce the potential immunogenicity of the antibody. This approach is also referred to as “deimmunization” and is described in further detail in U.S. Patent Publication No. 20030153043 by Carr et al.

In addition, or alternative to modifications made within the framework or CDR regions, antibodies of the invention may be engineered to include modifications within the Fc region, typically to alter one or more functional properties of the antibody, such as serum half-life, complement fixation, Fc receptor binding, and/or antigen-dependent cellular cytotoxicity. Furthermore, an antibody of the invention may be chemically modified (e.g., one or more chemical moieties can be attached to the antibody) or be modified to alter its glycosylation, again to alter one or more functional properties of the antibody. Each of these embodiments is described in further detail below. The numbering of residues in the Fc region is that of the EU index of Kabat.

In one embodiment, the hinge region of CH1 is modified such that the number of cysteine residues in the hinge region is altered, e.g., increased or decreased. This approach is described further in U.S. Pat. No. 5,677,425 by Bodmer et al. The number of cysteine residues in the hinge region of CH1 is altered to, for example, facilitate assembly of the light and heavy chains or to increase or decrease the stability of the antibody.

In another embodiment, the Fc hinge region of an antibody is mutated to decrease the biological half-life of the antibody. More specifically, one or more amino acid mutations are introduced into the CH2-CH3 domain interface region of the Fc-hinge fragment such that the antibody has impaired Staphylococcyl protein A (SpA) binding relative to native Fc-hinge domain SpA binding. This approach is described in further detail in U.S. Pat. No. 6,165,745 by Ward et al.

In another embodiment, the antibody is modified to increase its biological half-life. Various approaches are possible. For example, one or more of the following mutations can be introduced: T252L, T254S, T256F, as described in U.S. Pat. No. 6,277,375 to Ward. Alternatively, to increase the biological half-life, the antibody can be altered within the CH1 or CL region to contain a salvage receptor binding epitope taken from two loops of a CH2 domain of an Fc region of an IgG, as described in U.S. Pat. Nos. 5,869,046 and 6,121,022 by Presta et al. These strategies will be effective as long as the binding of the antibody to an antigen, e.g., ATIC, is not compromised.

In yet other embodiments, the Fc region is altered by replacing at least one amino acid residue with a different amino acid residue to alter the effector function(s) of the antibody. For example, one or more amino acids selected from amino acid residues 234, 235, 236, 237, 297, 318, 320 and 322 can be replaced with a different amino acid residue such that the antibody has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260, both by Winter et al.

In another example, one or more amino acids selected from amino acid residues 329, 331 and 322 can be replaced with a different amino acid residue such that the antibody has altered C1q binding and/or reduced or abolished complement dependent cytotoxicity (CDC). This approach is described in further detail in U.S. Pat. No. 6,194,551 by Idusogie et al.

In another example, one or more amino acid residues within amino acid positions 231 and 239 are altered to thereby alter the ability of the antibody to fix complement. This approach is described further in PCT Publication WO 94/29351 by Bodmer et al.

In yet another example, the Fc region is modified to increase the ability of the antibody to mediate antibody dependent cellular cytotoxicity (ADCC) and/or to increase the affinity of the antibody for an Fcγ receptor by modifying one or more amino acids at the following positions: 238, 239, 248, 249, 252, 254, 255, 256, 258, 265, 267, 268, 269, 270, 272, 276, 278, 280, 283, 285, 286, 289, 290, 292, 293, 294, 295, 296, 298, 301, 303, 305, 307, 309, 312, 315, 320, 322, 324, 326, 327, 329, 330, 331, 333, 334, 335, 337, 338, 340, 360, 373, 376, 378, 382, 388, 389, 398, 414, 416, 419, 430, 434, 435, 437, 438 or 439. This approach is described further in PCT Publication WO 00/42072 by Presta. Moreover, the binding sites on human IgG1 for FcγR1, FcγRII, FcγRIII and FcRn have been mapped and variants with improved binding have been described (see Shields et al. (2001) J. Biol. Chem. 276: 6591-6604). Specific mutations at positions 256, 290, 298, 333, 334 and 339 were shown to improve binding to FcγRIII. Additionally, the following combination mutants were shown to improve FcγRIII binding: T256A/S298A, S298A/E333A, S298A/K224A and S298A/E333A/K334A.

In still another embodiment, the C-terminal end of an antibody of the present invention is modified by the introduction of a cysteine residue as is described in U.S. Provisional Application Ser. No. 60/957,271, which is hereby incorporated by reference in its entirety. Such modifications include, but are not limited to, the replacement of an existing amino acid residue at or near the C terminus of a full-length heavy chain sequence, as well as the introduction of a cysteine-containing extension to the C terminus of a full-length heavy chain sequence. In preferred embodiments, the cysteine-containing extension comprises the sequence alanine-alanine-cysteine (from N-terminal to C-terminal).

In preferred embodiments the presence of such C-terminal cysteine modifications provide a location for conjugation of a partner molecule, such as a therapeutic agent or a marker molecule. In particular, the presence of a reactive thiol group, due to the C-terminal cysteine modification, can be used to conjugate a partner molecule employing the disulfide linkers described in detail below. Conjugation of the antibody to a partner molecule in this manner allows for increased control over the specific site of attachment. Furthermore, by introducing the site of attachment at or near the C terminus, conjugation can be optimized such that it reduces or eliminates interference with the antibody's functional properties, and allows for simplified analysis and quality control of conjugate preparations.

In still another embodiment, the glycosylation of an antibody is modified. For example, an aglycoslated antibody can be made (i.e., the antibody lacks glycosylation). Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Such aglycosylation may increase the affinity of the antibody for antigen. Such an approach is described in further detail in U.S. Pat. Nos. 5,714,350 and 6,350,861 to Co et al. Additional approaches for altering glycosylation are described in further detail in U.S. Pat. No. 7,214,775 to Hanai et al., U.S. Pat. No. 6,737,056 to Presta, U.S. Pub No. 20070020260 to Presta, PCT Publication No. WO/2007/084926 to Dickey et al., PCT Publication No. WO/2006/089294 to Zhu et al., and PCT Publication No. WO/2007/055916 to Ravetch et al., each of which is hereby incorporated by reference in its entirety.

Additionally, or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. For example, the cell lines Ms704, Ms705, and Ms709 lack the fucosyltransferase gene, FUT8 (alpha (1,6) fucosyltransferase), such that antibodies expressed in the Ms704, Ms705, and Ms709 cell lines lack fucose on their carbohydrates. The Ms704, Ms705, and Ms709 FUT8^{-/-} cell lines were created by the targeted disruption of the FUT8 gene in CHO/DG44 cells using two replacement vectors (see U.S. Patent Publication No. 20040110704 by Yamane et al. and Yamane-Ohnuki et al. (2004) Biotechnol. Bioeng. 87: 614-622). As another example, EP 1,176,195 by Hanai et al. describes a cell line with a functionally disrupted FUT8 gene, which encodes a fucosyl transferase, such that antibodies expressed in such a cell line exhibit hypofucosylation by reducing or eliminating the alpha 1,6 bond-related enzyme. Hanai et al. also describe cell lines which have a low enzyme activity for adding fucose to the N-acetylglucosamine that binds to the Fc region of the antibody or does not have the enzyme activity, for example the rat myeloma cell line YB2/0 (ATCC CRL 1662). PCT Publication WO 03/035835 by Presta describes a variant CHO cell line, Lec13 cells, with reduced ability to attach fucose to Asn(297)-linked carbohydrates, also resulting in hypofucosylation of antibodies expressed in that host cell (see also Shields et al. (2002) J. Biol. Chem. 277: 26733-26740). PCT Publication WO 99/54342 by Umana et al. describes cell lines engineered to express glycoprotein-modifying glycosyl transferases (e.g., beta(1,4)-N-acetylglucosaminyltransferase III (GnTIII)) such that antibodies expressed in the engineered cell lines exhibit increased bisecting GlcNac structures which results in increased ADCC activity of the antibodies (see also Umana et al. (1999) Nat. Biotech. 17: 176-180). Alternatively, the fucose residues of the antibody may be cleaved off using a fucosidase enzyme. For example, the fucosidase alpha-L-fucosidase removes fucosyl residues from antibodies (Tarentino et al. (1975) Biochem. 14: 5516-5523).

Additionally, or alternatively, an antibody can be made that has an altered type of glycosylation, wherein that alteration relates to the level of sialyation of the antibody. Such alterations are described in PCT Publication No. WO/2007/084926 to Dickey et al., and PCT Publication No. WO/2007/055916 to Ravetch et al., both of which are incoporated by reference in their entirety. For example, one may employ an enzymatic reaction with sialidase, such as, for example, Arthrobacter ureafacens sialidase. The conditions of such a reaction are generally described in the U.S. Pat. No. 5,831,077, which is hereby incorporated by reference in its entirety. Other non-limiting examples of suitable enzymes are neuraminidase and N-Glycosidase F, as described in Schloemer et al. (1975) J. Virol. 15, 882-893 and in Leibiger et al. (1999) Biochem. J. 338, 529-538, respectively. Desialylated antibodies may be further purified by using affinity chromatography. Alternatively, one may employ methods to increase the level of sialyation, such as by employing sialytransferase enzymes. Conditions of such a reaction are generally described in Basset et al. (2000) Scand. J. Immunol. 51: 307-311.

Another modification of the antibodies herein that is contemplated by the invention is pegylation. An antibody can be pegylated to, for example, increase the biological (e.g., serum) half-life of the antibody. To pegylate an antibody, the antibody, or antigen-binding fragment thereof, typically is reacted with polyethylene glycol (PEG), such as a reactive ester or aldehyde derivative of PEG, under conditions in which one or more PEG groups become attached to the antibody or antibody fragment. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C1-C10) alkoxy- or aryloxy-polyethylene glycol or polyethylene glycol-maleimide. In certain embodiments, the antibody to be pegylated is an aglycosylated antibody. Methods for pegylating proteins are known in the art and can be applied to the antibodies of the invention. See for example, EP 0 154 316 by Nishimura et al. and EP 0 401 384 by Ishikawa et al. As such, the methods of pegylation described here also apply to the peptidic molecules of the invention described below.

### Production of Antibodies of the Invention

Methods for producing antibodies are well-established. One skilled in the art will recognize that many procedures are available for the production of antibodies, for example, as described in Antibodies, A Laboratory Manual, Ed Harlow and David Lane, Cold Spring Harbor Laboratory (1988), Cold Spring Harbor, N.Y. One skilled in the art will also appreciate that binding fragments or Fab fragments which mimic antibodies can also be prepared from genetic information by various procedures (Antibody Engineering: A Practical Approach (Borrebaeck, C., ed.), 1995, Oxford University Press, Oxford; J. Immunol. 149, 3914-3920 (1992)). Monoclonal and polyclonal antibodies to molecules, e.g., proteins, and markers also commercially available (R and D Systems, Minneapolis, Minn.; HyTest, HyTest Ltd., Turku Finland; Abcam Inc., Cambridge, Mass., USA, Life Diagnostics, Inc., West Chester, Pa., USA; Fitzgerald Industries International, Inc., Concord, Mass. 01742-3049 USA; BiosPacific, Emeryville, Calif.).

In some embodiments, the antibody is a polyclonal antibody. In other embodiments, the antibody is a monoclonal antibody.

Polyclonal antibodies of the present invention can be produced by a variety of techniques that are well known in the art. Polyclonal antibodies are derived from different B-cell lines and thus may recognize multiple epitopes on the same antigen. Polyclonal antibodies are typically produced by immunization of a suitable mammal with the antigen of interest, e.g., ATIC. Animals often used for production of polyclonal antibodies are chickens, goats, guinea pigs, hamsters, horses, mice, rats, sheep, and, most commonly, rabbit. Standard methods to produce polyclonal antibodies are widely known in the art and can be combined with the methods of the present invention (e.g., U.S. Pat. Nos. 4,719,290, 6,335,163, 5,789,208, 2,520,076, 2.543.215, and 3.597.409, the entire contents of which are incorporated herein by reference.

Monoclonal antibodies of the present invention can be produced by any of a variety of techniques known to those of ordinary skill in the art (see, for example, Harlow and Lane, Antibodies: A Laboratory Manual, Cold Spring Harbor Laboratory, 1988). In general, antibodies can be produced by cell culture techniques, including the generation of monoclonal antibodies as described herein, or via transfection of antibody genes into suitable bacterial or mammalian cell hosts, in order to allow for the production of recombinant antibodies.

Monoclonal antibodies may be prepared using hybridoma methods, such as the technique of Kohler and Milstein (Eur. J. Immunol. 6:511-519, 1976), and improvements thereto. These methods involve the preparation of immortal cell lines capable of producing antibodies having the desired specificity. Monoclonal antibodies may also be made by recombinant DNA methods, such as those described in U.S. Pat. No. 4,816,567. DNA encoding antibodies employed in the disclosed methods may be isolated and sequenced using conventional procedures. Recombinant antibodies, antibody fragments, and/or fusions thereof, can be expressed in vitro or in prokaryotic cells (e.g., bacteria) or eukaryotic cells (e.g., yeast, insect or mammalian cells) and further purified as necessary using well known methods.

More particularly, monoclonal antibodies (MAbs) may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified expressed protein, polypeptide or peptide. The immunizing composition is administered in a manner effective to stimulate antibody producing cells. The methods for generating monoclonal antibodies (MAbs) generally begin along the same lines as those for preparing polyclonal antibodies. Rodents such as mice and rats are preferred animals, however, the use of rabbit, sheep or frog cells is also possible. The use of rats may provide certain advantages (Goding, 1986, pp. 60-61), but mice are preferred, with the BALB/c mouse being most preferred as this is most routinely used and generally gives a higher percentage of stable fusions.

The animals are injected with antigen as described above. The antigen may be coupled to carrier molecules such as keyhole limpet hemocyanin if necessary. The antigen would typically be mixed with adjuvant, such as Freund's complete or incomplete adjuvant. Booster injections with the same antigen would occur at approximately two-week intervals. Following immunization, somatic cells with the potential for producing antibodies, specifically B lymphocytes (B cells), are selected for use in the MAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible. Often, a panel of animals will have been immunized and the spleen of the animal with the highest antibody titer will be removed and the spleen lymphocytes obtained by homogenizing the spleen with a syringe.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

The selected hybridomas would then be serially diluted and cloned into individual antibody-producing cell lines, which clones may then be propagated indefinitely to provide MAbs. The cell lines may be exploited for MAb production in two basic ways. A sample of the hybridoma may be injected (often into the peritoneal cavity) into a histocompatible animal of the type that was used to provide the somatic and myeloma cells for the original fusion. The injected animal develops tumors secreting the specific monoclonal antibody produced by the fused cell hybrid. The body fluids of the animal, such as serum or ascites fluid, may then be tapped to provide MAbs in high concentration. The individual cell lines also may be cultured in vitro, where the MAbs are naturally secreted into the culture medium from which they may be readily obtained in high concentrations. MAbs produced by either means may be further purified, if desired, using filtration, centrifugation and various chromatographic methods such as HPLC or affinity chromatography.

Large amounts of the monoclonal antibodies of the present invention also may be obtained by multiplying hybridoma cells in vivo. Cell clones are injected into mammals which are histocompatible with the parent cells, e.g., syngeneic mice, to cause growth of antibody-producing tumors. Optionally, the animals are primed with a hydrocarbon, especially oils such as pristane (tetramethylpentadecane) prior to injection.

In accordance with the present invention, fragments of the monoclonal antibody of the invention may be obtained from the monoclonal antibody produced as described above, by methods which include digestion with enzymes such as pepsin or papain and/or cleavage of disulfide bonds by chemical reduction. Alternatively, monoclonal antibody fragments encompassed by the present invention may be synthesized using an automated peptide synthesizer.

Antibodies may also be derived from a recombinant antibody library that is based on amino acid sequences that have been designed in silico and encoded by polynucleotides that are synthetically generated. Methods for designing and obtaining in silico-created sequences are known in the art (Knappik et al., J. Mol. Biol. 296:254:57-86, 2000; Krebs et al., J. Immunol. Methods 254:67-84, 2001; U.S. Pat. No. 6,300,064).

Digestion of antibodies to produce antigen-binding fragments thereof can be performed using techniques well known in the art. For example, the proteolytic enzyme papain preferentially cleaves IgG molecules to yield several fragments, two of which (the “F(ab)” fragments) each comprise a covalent heterodimer that includes an intact antigen-binding site. The enzyme pepsin is able to cleave IgG molecules to provide several fragments, including the “F(ab′).sub.2” fragment, which comprises both antigen-binding sites. “Fv” fragments can be produced by preferential proteolytic cleavage of an IgM, IgG or IgA immunoglobulin molecule, but are more commonly derived using recombinant techniques known in the art. The Fv fragment includes a non-covalent V_{H}::V_{L} heterodimer including an antigen-binding site which retains much of the antigen recognition and binding capabilities of the native antibody molecule (Inbar et al., Proc. Natl. Acad. Sci. USA 69:2659-2662 (1972); Hochman et al., Biochem. 15:2706-2710 (1976); and Ehrlich et al., Biochem. 19:4091-4096 (1980)).

Antibody fragments that specifically bind to the protein biomarkers disclosed herein can also be isolated from a library of scFvs using known techniques, such as those described in U.S. Pat. No. 5,885,793.

A wide variety of expression systems are available in the art for the production of antibody fragments, including Fab fragments, scFv, VL and VHs. For example, expression systems of both prokaryotic and eukaryotic origin may be used for the large-scale production of antibody fragments. Particularly advantageous are expression systems that permit the secretion of large amounts of antibody fragments into the culture medium. Eukaryotic expression systems for large-scale production of antibody fragments and antibody fusion proteins have been described that are based on mammalian cells, insect cells, plants, transgenic animals, and lower eukaryotes. For example, the cost-effective, large-scale production of antibody fragments can be achieved in yeast fermentation systems. Large-scale fermentation of these organisms is well known in the art and is currently used for bulk production of several recombinant proteins.

Following screening and sequencing, antibodies may be produced using recombinant methods and compositions, e.g., as described in U.S. Pat. No. 4,816,567, incorporated by reference herein. An isolated nucleic acid encoding, for example, an anti-ATIC antibody is used to transform host cells for expression. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g., a Chinese Hamster Ovary (CHO) cell or lymphoid cell (e.g., Y0, NS0, Sp20 cell).

For recombinant production of an anti-ATIC antibody, a nucleic acid encoding an antibody is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TR1 cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR.sup.-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

Antibodies, or antigen binding fragments thereof, described herein are capable of binding to target proteins, such as ATIC, thereby inhibiting expression and/or activity of ATIC, and decreasing the expression of LRRK2. In some instances, antibodies, or antigen binding fragments thereof, described herein can inhibit the expression and/or activity of ATIC by at least 20%, e.g., 30%, 40%, 50%, 60%, 70%, 80%, 90%, 95%, or higher. In some instances, antibodies described herein can decrease LRRK2 expression levels by at least 20%, e.g., 30%, 40%, 50%, 60%, 70%, 80%, 90%, 95%, or higher.

### Small Molecules

In another aspect of the invention, the agent that decreases the expression of LRRK2, e.g., an inhibitor of ATIC, is a small molecule.

The small molecules of the instant invention are characterized by particular functional features or properties. For example, the small molecules bind to ATIC, or any other protein that interacts with ATIC, thereby inhibiting ATIC activity.

The terms “small molecule compounds”, “small molecule drugs”, “small molecules”, or “small molecule inhibitors” are used interchangeably herein to refer to the compounds of the present invention screened for an effect on ATIC and their ability to inhibit the activity of ATIC. These compounds may comprise compounds in PubChem database (pubchem.ncbi.nlm.nih.gov/), the Molecular Libraries Screening Center Network (MLSCN) database, compounds in related databases, or derivatives and/or functional analogues thereof.

As used herein, “analogue” or “functional analogue” refers to a chemical compound or small molecule inhibitor that is structurally similar to a parent compound, but differs slightly in composition (e.g., one or more atoms or functional groups are added, removed, or modified). The analogue may or may not have different chemical or physical properties than the original compound and may or may not have improved biological and/or chemical activity. For example, the analogue may be more hydrophobic or it may have altered activity (increased, decreased, or identical to parent compound) as compared to the parent compound. The analogue may be a naturally or non-naturally occurring (e.g., recombinant) variant of the original compound. Other types of analogues include isomers (enantiomers, diasteromers, and the like) and other types of chiral variants of a compound, as well as structural isomers. The analogue may be a branched or cyclic variant of a linear compound. For example, a linear compound may have an analogue that is branched or otherwise substituted to impart certain desirable properties (e.g., improve hydrophilicity or bioavailability).

As used herein, “derivative” refers to a chemically or biologically modified version of a chemical compound or small molecule inhibitor that is structurally similar to a parent compound and (actually or theoretically) derivable from that parent compound. A “derivative” differs from an “analogue” or “functional analogue” in that a parent compound may be the starting material to generate a “derivative,” whereas the parent compound may not necessarily be used as the starting material to generate an “analogue” or “functional analogue.” A derivative may or may not have different chemical or physical properties of the parent compound. For example, the derivative may be more hydrophilic or it may have altered reactivity as compared to the parent compound. Derivatization (i.e., modification by chemical or other means) may involve substitution of one or more moieties within the molecule (e.g., a change in functional group). For example, a hydrogen may be substituted with a halogen, such as fluorine or chlorine, or a hydroxyl group (—OH) may be replaced with a carboxylic acid moiety (—COOH). The term “derivative” also includes conjugates, and prodrugs of a parent compound (i.e., chemically modified derivatives which can be converted into the original compound under physiological conditions). For example, the prodrug may be an inactive form of an active agent. Under physiological conditions, the prodrug may be converted into the active form of the compound. Prodrugs may be formed, for example, by replacing one or two hydrogen atoms on nitrogen atoms by an acyl group (acyl prodrugs) or a carbamate group (carbamate prodrugs). More detailed information relating to prodrugs is found, for example, in Fleisher et al. (1996) Adv. Drug Deliv. Rev. 19: 115; Design of Prodrugs, H. Bundgaard (ed.), Elsevier, 1985; and H. Bundgaard, Drugs of the Future 16 (1991) 443. The term “derivative” is also used to describe all solvates, for example hydrates or adducts (e.g., adducts with alcohols), active metabolites, and salts of the parent compound. The type of salt that may be prepared depends on the nature of the moieties within the compound. For example, acidic groups such as carboxylic acid groups can form alkali metal salts or alkaline earth metal salts (e.g., sodium salts, potassium salts, magnesium salts, calcium salts, and salts with physiologically tolerable quaternary ammonium ions and acid addition salts with ammonia and physiologically tolerable organic amines such as triethylamine, ethanolamine, or tris-(2-hydroxyethyl)amine). Basic groups can form acid addition salts, for example with inorganic acids such as hydrochloric acid (“HCl”), sulfuric acid, or phosphoric acid, or with organic carboxylic acids and sulfonic acids such as acetic acid, citric acid, benzoic acid, maleic acid, fumaric acid, tartaric acid, methanesulfonic acid, or p-toluenesulfonic acid. Compounds that simultaneously contain a basic group and an acidic group such as a carboxyl group in addition to basic nitrogen atoms can be present as zwitterions. Salts can be obtained by customary methods known to those skilled in the art, for example, by combining a compound with an inorganic or organic acid or base in a solvent or diluent, or from other salts by cation exchange or anion exchange.

Small molecules are known to have molecular weights of 1200 or below, 1000 or below, 900 or below, 800 or below, 700 or below, 600 or below, 500 or below, 400 or below, 300 or below, 200 or below, 100 or below, 50 or below, 25 or below, or 10 or below.

The small molecules of the present invention are selected or designed to bind to ATIC, or any other protein that interacts with ATIC.

In preferred embodiments, a small molecule of the invention binds to ATIC, or any other protein that interacts with ATIC with high affinity, for example, with an affinity of a K_{D} of 1×10⁻⁷ M or less, a K_{D} of 5×10⁻⁸ M or less, a K_{D} of 1×10⁻⁸ M or less, a K_{D} of 5×10⁻⁹ M or less, or a K_{D} of between 1×10⁻⁸ M and 1×10⁻¹⁰ M or less.

Small molecules of the invention may be made or selected by several methods known in the art and by methods as described herein. Screening procedures can be used to identify small molecules from libraries which bind ATIC, or any other protein that interacts with ATIC.

In some embodiments, the ATIC inhibitors are antifolates, which were used historically to modulate cellular proliferation (e.g., neoplastic treatments) and immune reactions. Exemplary antifolate inhibitors include, but are not limited to: piritrexim, ZD1694, lometrexol, edatrexate, trimertexate and methotrexate.

A variety of agents are known to inhibit ATIC activity. Such agents are disclosed, e.g., in WO 00/13688 (Agouron/Pfizer); U.S. Pat. No. 6,323,210; Marsilje et al., Bioorg. Med. Chem. 11:4503 (2003); Tatlock et al. (Agouron/Pfizer), 217th Am. Chem. Soc. Meeting, Anaheim, Calif. March 1999; Cheong C. G. et al., J. Biol. Chem. 279(17):18034-45 (2004); Acid Yellow 54 (Xu, L et al, J. Biol. Chem., 279(48):50555-65 (2004); Isolates from the NCI Database (Li, C et al, J. Med. Chem., 47(27):6681-90 (2004)). Any one of these ATIC inhibitory agents may also be used according to the methods of the invention.

### Peptidic Molecules

In another aspect of the invention, the agent that decreases the expression of LRRK2, e.g., an ATIC inhibitor, is a peptidic molecule.

In one embodiment, the peptidic moieties of the invention may comprise an entire protein domain of ATIC. In some embodiments, the peptidic moieties of the invention may have as little as 50% identity to ATIC, e.g., a peptidic moiety of the invention may be at least 50% identical, at least 60% identical, at least 70% identical, at least 80% identical, at least 90% identical, or at least 95%, 96%, 97%, or 98% identical to ATIC.

In another embodiment, the peptidic moieties of the invention may comprise an entire protein domain of any other protein that interacts with ATIC. In some embodiments, the peptidic moieties of the invention may have as little as 50% identity to the protein that interacts with ATIC, for example, a peptidic moiety of the invention may be at least 50% identical, at least 60% identical, at least 70% identical, at least 80% identical, at least 90% identical, or at least 95%, 96%, 97%, or 98% identical to the protein that interacts with ATIC.

A peptidic moiety of the invention may bind to contiguous or non-contiguous amino acid residues of ATIC, or any other protein that interacts with ATIC.

A peptide molecule of the invention may be further modified to increase its stability, bioavailability or solubility. For example, one or more L-amino acid residues within the peptidic molecules may be replaced with a D-amino acid residue. The term “mimetic” as applied to the peptidic molecules of the present invention is intended to include molecules that mimic the chemical structure of a D-peptidic structure and retain the functional properties of the D-peptidic structure. The term “mimetic” is further intended to encompass an “analogue” and/or “derivative” of a peptide as described below. Approaches to designing peptide analogs, derivatives and mimetics are known in the art. For example, see Farmer, P. S. in Drug Design (E. J. Ariens, ed.) Academic Press, New York, 1980, vol. 10, pp. 119-143; Ball and Alewood (1990) J. Mol. Recognition 3: 55; Morgan and Gainor (1989) Ann. Rep. Med. Chem. 24: 243; and Freidinger (1989) Trends Pharmacol. Sci. 10: 270. See also Sawyer (1995) “Peptidomimetic Design and Chemical Approaches to Peptide Metabolism” in Taylor, M. D. and Amidon, G. L. (eds.) Peptide-Based Drug Design: Controlling Transport and Metabolism, Chapter 17; Smith et al. (1995) J. Am. Chem. Soc. 117: 11113-11123; Smith et al. (1994) J. Am. Chem. Soc. 116: 9947-9962; and Hirschman et al. (1993) J. Am. Chem. Soc. 115: 12550-12568.

Other methods to stabilize peptides and peptide structures may be used, e.g., olefinic cross-linking of helices through O-allyl serine residues (Blackwell, H. E.; Grubbs, R. H. Angew. Chem., Int. Ed. 1998, 37, 3281-3284, incorporated herein by reference), all-hydrocarbon cross-linking (Schafmeister and Verdine J. Am. Chem. Soc. 2000, 122 (24), 5891-5892, incorporated herein by reference) and the methods disclosed in U.S. Pat. No. 7,183,059 (incorporated herein by reference). The methods disclosed in Blackwell et al. and Schafmeister et al. may be described as producing “stapled” peptides, i.e., peptides which are covalently locked into a particular conformational state or secondary structure, or peptides which have a particular intramolecular covalent linkage which predisposes them to form a particular conformation or structure. If a peptide thus treated is predisposed to, e.g., form an alpha-helix which is important for target binding, then the energetic threshold for binding will be lowered. Such “stapled” peptides have been shown to be resistant to proteases and may also be designed to cross the cellular membrane more effectively (also see Walensky et al. Science 2004: Vol. 305. no. 5689, pp. 1466-1470; Bernal et al. J Am Chem Soc. 2007, 129(9):2456-7 which are incorporated herein by reference). Accordingly, peptides of the invention may be thus stapled or otherwise modified to lock them into a specific conformational shape or they may be modified to be predisposed to particular conformation or secondary structure which is beneficial for binding. It is contemplated that such peptide modifications may occur prior to peptide selection such that the benefit of any conformational constraints may also be selected for. Alternatively, in some embodiments, the modifications may be made after selection to preserve a conformation known to be beneficial to binding or to further enhance a peptide candidate. See also, WO/2010/033617, the entire contents of which are incorporated herein by reference.

Other methods to stabilize peptides and peptide structures include linking the amino and carboxy termini of a protein with a peptide bond to form a circular or cyclic peptide. See, e.g., WO/2008/07489 and U.S. Pat. No. 55,726,287, the entire contents of each of which are incorporated herein by reference.

As used herein, a “derivative” of a peptidic molecule of the invention refers to a form of the peptidic molecule in which one or more reaction groups on the molecule have been derivatized with a substituent group. Examples of peptide derivatives include peptides in which an amino acid side chain, the peptide backbone, or the N or C terminus has been derivatized (e.g., peptidic compounds with methylated amide linkages). As used herein an “analogue” of a peptidic molecule of the invention refers to a peptidic molecule that retains chemical structures of the molecule necessary for functional activity of the molecule yet also contains certain chemical structures that differ from the molecule. An example of an analogue of a naturally-occurring peptide is a peptide that includes one or more non-naturally-occurring amino acids. As used herein, a “mimetic” of a peptidic molecule of the invention refers to a peptidic molecule in which chemical structures of the molecule necessary for functional activity of the molecule have been replaced with other chemical structures that mimic the conformation of the molecule. Examples of peptidomimetics include peptidic compounds in which the peptide backbone is substituted with one or more benzodiazepine molecules (see e.g., James et al. (1993) Science 260:1937-1942).

Analogues of the peptidic molecules of the invention are intended to include molecules in which one or more L- or D-amino acids of the peptidic structure are substituted with a homologous amino acid such that the properties of the molecule are maintained. Preferably conservative amino acid substitutions are made at one or more amino acid residues. A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Non-limiting examples of homologous substitutions that can be made in the structures of the peptidic molecules of the invention include substitution of D-phenylalanine with D-tyrosine, D-pyridylalanine or D-homophenylalanine, substitution of D-leucine with D-valine or other natural or non-natural amino acid having an aliphatic side chain and/or substitution of D-valine with D-leucine or other natural or non-natural amino acid having an aliphatic side chain.

The term “mimetic,” and in particular, “peptidomimetic,” is intended to include isosteres. The term “isostere” as used herein is intended to include a chemical structure that can be substituted for a second chemical structure because the steric conformation of the first structure fits a binding site specific for the second structure. The term specifically includes peptide back-bone modifications (i.e., amide bond mimetics) well known to those skilled in the art. Such modifications include modifications of the amide nitrogen, the α-carbon, amide carbonyl, complete replacement of the amide bond, extensions, deletions or backbone crosslinks. Several peptide backbone modifications are known, including ψ[CH₂S], ψ[CH₂NH], ψ[CSNH₂], ψ[NHCO], ψ[COCH₂], and ψ[(E) or (Z) CH═CH]. In the nomenclature used above, ψ indicates the absence of an amide bond. The structure that replaces the amide group is specified within the brackets.

Other possible modifications include an N-alkyl (or aryl) substitution (ψ[CONR]), or backbone crosslinking to construct lactams and other cyclic structures. Other derivatives of the modulator compounds of the invention include C-terminal hydroxymethyl derivatives, O-modified derivatives (e.g., C-terminal hydroxymethyl benzyl ether), N-terminally modified derivatives including substituted amides such as alkylamides and hydrazides.

Peptidic molecules of the present invention may be made by standard methods known in the art. The peptidic molecule may be cloned from human cells using standard techniques, inserted in to a recombinant vector, and expressed in an in vitro cell system (e.g., by transfection of the vector into yeast cells). Alternatively, the peptidic molecules may be designed and synthesized de novo via known synthesis methods such as Atherton et al. (1989) Oxford, England: IRL Press. ISBN 0199630674; Stewart et al. (1984) 2nd edition, Rockford: Pierce Chemical Company, 91. ISBN 0935940030; Merrifield (1963) J. Am. Chem. Soc. 85: 2149-2154.

The peptidic molecules can then be tested for functional activity using any of the assays described herein, e.g., those described in the Examples section below.

### Activating Agents

In some embodiments, the agent that decreases the expression of LRRK2 is an agent that activate or induce LRRK2 mRNA decay. In some embodiments, the agent increases the expression and/or activity of AU-rich element RNA binding protein 1 (AUF1). In some embodiments, the agent increases the expression and/or activity of a decapping enzyme involving in the process of mRNA decay, e.g., mRNA decapping enzyme 1 (DCP1) and/or mRNA decapping enzyme 2 (DCP2).

In some embodiments, the agents include any compound or molecule that can regulate the expression and/or activity of AUF1, DCP1 and/or DCP2, for example, the mRNA expression and/or protein expression of AUF1, DCP1 and/or DCP2; the mRNA and/or protein stability of AUF1, DCP1 and/or DCP2; and/or the biological activity of AUF1, DCP1 and/or DCP2. A modulator can modulate the expression and/or activity of AUF1 either directly or indirectly, e.g., through another molecules, e.g., a binding partner of AUF1, DCP1 and/or DCP2.

Examples of such activating agents include proteins, nucleic acid molecules, e.g., expression vectors comprising nucleic acid molecules, agonist antibodies, or antigen-binding fragment thereof, and small molecules that stimulate expression and/or activity of AUF1, DCP1 and/or DCP2.

In some embodiments, the agent that increases the expression and/or activity of AUF1 is selected from the group consisting of a small molecule activator of AUF1, an agonist antibody of AUF1, or antigen-binding fragment thereof, an AUF1 protein or functional fragment thereof, a nucleic acid encoding the AUF1 protein or functional fragment thereof, or a protein and a nucleic acid that activates the transcription and/or translation of AUF1.

In some embodiments, the agent that increases the expression and/or activity of DCP1 and/or DCP2 is selected from the group consisting of a small molecule activator of DCP1 and/or DCP2, an agonist antibody of DCP1 and/or DCP2, or antigen-binding fragment thereof, a DCP1 and/or DCP2 protein or functional fragment thereof, a nucleic acid encoding the AUF1 protein or functional fragment thereof, or a protein and a nucleic acid that activates the transcription and/or translation of AUF1.

### IV. Pharmaceutical Compositions

The present disclosure also includes pharmaceutical compositions and formulations which include the agents that decrease the expression of LRRK2 of the disclosure. In one embodiment, provided herein are pharmaceutical compositions containing an agent that decreases the expression of LRRK2, e.g., an agent that induces LRRK2 mRNA decay, e.g., an ATIC inhibitor, an ATIC substrate (e.g., AICAR/ZMP), a precursor of ATIC substrate (e.g., AICAr), an agent that increases the expression and/or activity of AUF1, or an agent that increases the expression and/or activity of DCP1 and/or DCP2, as described herein, and a pharmaceutically acceptable carrier. The pharmaceutical compositions containing the agent are useful for treating a subject who would benefit from decreasing the expression of LRRK2, e.g., a subject having an LRRK2-associated disorder or condition, e.g., Parkinson's Disease.

The pharmaceutical compositions of the present disclosure can be administered in a number of ways depending upon whether local or systemic treatment is desired and upon the area to be treated. Administration can be oral, parental, topical (e.g., by a transdermal patch), intranasal, intratracheal, epidermal and transdermal.

Parenteral administration includes intravenous, intraarterial, subcutaneous, intraperitoneal or intramuscular injection or infusion; subdermal, e.g., via an implanted device; or intracranial, e.g., by intraparenchymal, intrathecal or intraventricular, administration. Parenteral administration may be by continuous infusion over a selected period of time.

Pharmaceutical compositions and formulations for topical administration can include transdermal patches, ointments, lotions, creams, gels, drops, suppositories, sprays, liquids and powders. Conventional pharmaceutical carriers, aqueous, powder or oily bases, thickeners and the like can be necessary or desirable. Coated condoms, gloves and the like can also be useful. Suitable topical formulations include those in which the oligonucleotides featured in the disclosure are in admixture with a topical delivery agent such as lipids, liposomes, fatty acids, fatty acid esters, steroids, chelating agents and surfactants. Suitable lipids and liposomes include neutral (e.g., dioleoylphosphatidyl DOPE ethanolamine, dimyristoylphosphatidyl choline DMPC, distearolyphosphatidyl choline) negative (e.g., dimyristoylphosphatidyl glycerol DMPG) and cationic (e.g., dioleoyltetramethylaminopropyl DOTAP and dioleoylphosphatidyl ethanolamine DOTMA). Oligonucleotides featured in the disclosure can be encapsulated within liposomes or can form complexes thereto, in particular to cationic liposomes. Alternatively, oligonucleotides can be complexed to lipids, in particular to cationic lipids. Suitable fatty acids and esters include but are not limited to arachidonic acid, oleic acid, eicosanoic acid, lauric acid, caprylic acid, capric acid, myristic acid, palmitic acid, stearic acid, linoleic acid, linolenic acid, dicaprate, tricaprate, monoolein, dilaurin, glyceryl 1-monocaprate, 1-dodecylazacycloheptan-2-one, an acylcarnitine, an acylcholine, or a C1-20 alkyl ester (e.g., isopropylmyristate IPM), monoglyceride, diglyceride or pharmaceutically acceptable salt thereof. Topical formulations are described in detail in U.S. Pat. No. 6,747,014, which is incorporated herein by reference.

Compositions and formulations for parenteral, intraparenchymal (into the brain), intrathecal, intraventricular or intrahepatic administration can include sterile aqueous solutions which can also contain buffers, diluents and other suitable additives such as, but not limited to, penetration enhancers, carrier compounds and other pharmaceutically acceptable carriers or excipients.

Useful solutions for oral or parenteral administration can be prepared by any of the methods well known in the pharmaceutical art, described, for example; in Remington's Pharmaceutical Sciences, 18th ed. (Mack Publishing Company, 1990). The parenteral preparation can be enclosed in ampoules, disposable syringes or multiple dose vials made of glass or plastic. Formulations also can include, for example, polyalkylene glycols such as polyethylene glycol, oils of vegetable origin, and hydrogenated naphthalenes. Other potentially useful parenteral carriers for these drugs include ethylene-vinyl acetate copolymer particles, osmotic pumps, implantable infusion systems, and liposomes.

Formulations of the present disclosure suitable for oral administration may be in the form of: discrete units such as capsules, gelatin capsules, sachets, tablets, troches, or lozenges, each containing a predetermined amount of the drug; a powder or granular composition; a solution or a suspension in an aqueous liquid or non-aqueous liquid; or an oil-in-water emulsion or a water-in-oil emulsion. The drug may also be administered in the form of a bolus, electuary or paste. A tablet may be made by compressing or molding the drug optionally with one or more accessory ingredients. Compressed tablets may be prepared by compressing, in a suitable machine, the drug in a free-flowing form such as a powder or granules, optionally mixed by a binder, lubricant, inert diluent, surface active or dispersing agent. Molded tablets may be made by molding; in a suitable machine; a mixture of the powdered drug and suitable carrier moistened with an inert liquid diluent.

Oral compositions generally include an inert diluent or an edible carrier. For the purpose of oral therapeutic administration, the active compound can be incorporated with excipients. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose; a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). It should be stable under the conditions of manufacture and storage and should be preserved against the contaminating action of microorganisms such as bacteria and fungi. The carrier can be a solvent or dispersion medium containing, for example, water, ethanol, polyol (for example, glycerol, propylene glycol, and liquid polyethylene glycol), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, and/or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filter sterilization. Generally, dispersions are prepared by incorporating the active compound into a sterile vehicle which contains a basic dispersion medium and the required other ingredients from those enumerated above. In the case of sterile powders for the preparation of sterile injectable solutions; methods of preparation include vacuum drying and freeze-drying which yields a powder of the active ingredient plus any additional desired ingredient from a previously sterile-filtered solution thereof.

Formulations suitable for intra-articular administration may be in the form of a sterile aqueous preparation of the drug that may be in microcrystal line form, for example, in the form of an aqueous microcrystalline suspension. Liposomal formulations or biodegradable polymer systems may also be used to present the drug for both intra-articular and ophthalmic administration.

Systemic administration also can be by transmucosal or transdermal means. For transmucosal or transdermal administration, penetrants appropriate to the barrier to be permeated are used in the formulation. Such penetrants generally are known in the art, and include, for example, for transmucosal administration, detergents and bile salts. Transmucosal administration can be accomplished through the use of nasal sprays or suppositories. For transdermal administration, the active compounds typically are formulated into ointments, salves, gels, or creams as generally known in the art.

The active compounds may be prepared with carriers that will protect the compound against rapid elimination from the body, such as a controlled release formulation, including implants and microencapsulated delivery systems. Biodegradable, biocompatible polymers can be used; such as ethylene vinyl acetate, polyanhydrides, polyglycolic acid, collagen, polyorthoesters, and polylactic acid. Methods for preparation of such formulations will be apparent to those skilled in the art. Liposomal suspensions can also be used as pharmaceutically acceptable carriers. These can be prepared according to methods known to those skilled in the art, for example, as described in U.S. Pat. No. 4,522,811.

Oral or parenteral compositions can be formulated in dosage unit form for ease of administration and uniformity of dosage. Dosage unit form refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved, and the limitations inherent in the art of compounding such an active compound for the treatment of individuals. Furthermore, administration can be by periodic injections of a bolus, or can be made more continuous by intravenous, intramuscular or intraperitoneal administration from an external reservoir (e.g., an intravenous bag).

Where the active compound is to be used as part of a transplant procedure, it can be provided to the living tissue or organ to be transplanted prior to removal of tissue or organ from the donor. The compound can be provided to the donor host. Alternatively, or in addition, once removed from the donor, the organ or living tissue can be placed in a preservation solution containing the active compound. In all cases, the active compound can be administered directly to the desired tissue, as by injection to the tissue, or it can be provided systemically, either by oral or parenteral administration, using any of the methods and formulations described herein and/or known in the art. Where the drug comprises part of a tissue or organ preservation solution, any commercially available preservation solution can be used to advantage. For example, useful solutions known in the art include Collins solution, Wisconsin solution, Belzer solution, Eurocollins solution and lactated Ringer's solution.

The pharmaceutical formulations of the present disclosure, which can conveniently be presented in unit dosage form, can be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions of the present disclosure can be formulated into any of many possible dosage forms such as, but not limited to, tablets, capsules, gel capsules, liquid syrups, soft gels, suppositories, and enemas. The compositions of the present disclosure can also be formulated as suspensions in aqueous, non-aqueous or mixed media. Aqueous suspensions can further contain substances which increase the viscosity of the suspension including, for example, sodium carboxymethylcellulose, sorbitol or dextran. The suspension can also contain stabilizers.

The compositions of the present disclosure can also be prepared and formulated in additional formulations, such as emulsions or microemulsions, or be incorporated into a particle, e.g., a microparticle, which can be produced by spray-drying, or other methods including lyophilization, evaporation, fluid bed drying, vacuum drying, or a combination of these techniques. Penetration enhancers, e.g., surfactants, fatty acids, bile salts, chelating agents, and non-chelating non-surfactants, may be added in order to effect the efficient delvery of the compositions of the present disclosure, e.g., the delivery of the oligonucleotides, to the subject. Agents that enhance uptake of oligonucletide agents at the cellular level can also be added to the pharmaceutical and other compositions of the present disclosure, such as, cationic lipids, e.g., lipofectin, cationic glycerol derivatives, and polycationic molecules, e.g., polylysine.

The pharmaceutical compositions of this invention may also be administered using microspheres, liposomes, other microparticulate delivery systems or sustained release formulations placed in, near, or otherwise in communication with affected tissues or the bloodstream. Suitable examples of sustained release carriers include semipermeable polymer matrices in the form of shaped articles such as suppositories or microcapsules. Implantable or microcapsular sustained release matrices include polylactides (U.S. Pat. No. 3,773,319; EP 58,481), copolymers of L-glutamic acid and gamma ethyl-L-glutamate (Sidman et al., Biopolymers, 22, pp. 547-56 (1985)); poly(2-hydroxyethyl-methacrylate) or ethylene vinyl acetate (Langer et al., J. Biomed. Mater. Res., 15, pp. 167-277 (1981); Langer, Chem. Tech., 12, pp. 98-105 (1982)).

The pharmaceutical composition of the present disclosure may also include a pharmaceutical carrier or excipient. A pharmaceutical carrier or excipient is a pharmaceutically acceptable solvent, suspending agent or any other pharmacologically inert vehicle for delivering one or more nucleic acids to an animal. The excipient can be liquid or solid and is selected, with the planned manner of administration in mind, so as to provide for the desired bulk, consistency, etc., when combined with a nucleic acid and the other components of a given pharmaceutical composition. Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, talc, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc).

Formulations for topical administration of nucleic acids can include sterile and non-sterile aqueous solutions, non-aqueous solutions in common solvents such as alcohols, or solutions of the nucleic acids in liquid or solid oil bases. The solutions can also contain buffers, diluents and other suitable additives. Pharmaceutically acceptable organic or inorganic excipients suitable for non-parenteral administration which do not deleteriously react with nucleic acids can be used. Suitable pharmaceutically acceptable excipients include, but are not limited to, water, salt solutions, alcohol, polyethylene glycols, gelatin, lactose, amylose, magnesium stearate, talc, silicic acid, viscous paraffin, hydroxymethylcellulose, polyvinylpyrrolidone and the like.

Toxicity and therapeutic efficacy of the compositions can be determined by standard pharmaceutical procedures in cell cultures or experimental animals, e.g., for determining the LD₅₀ (the dose lethal to 50% of the population) and the ED₅₀ (the dose therapeutically effective in 50% of the population). Compounds that exhibit high therapeutic indices are preferred. The data obtained from cell culture assays and animal studies can be used in formulating a range of dosage for use in humans.

The dosage of the compositions (e.g., a composition including an oligonucleotide) described herein, can vary depending on many factors, such as the pharmacodynamic properties of the compound; the mode of administration; the age, health, and weight of the recipient; the nature and extent of the symptoms; the frequency of the treatment, and the type of concurrent treatment, if any; and the clearance rate of the compound in the animal to be treated. One of skill in the art can determine whether to administer the composition and tailor the appropriate dosage and/or therapeutic regimen of treatment with the composition based on the above factors. The compositions described herein may be administered initially in a suitable dosage that may be adjusted as required, depending on the clinical response. In some embodiments, the dosage of a composition (e.g., a composition including an oligonucleotide) is a prophylactically or a therapeutically effective amount. In some embodiments, treatment of a subject with a therapeutically effective amount of a composition can include a single treatment or a series of treatments. In addition, it is to be understood that the initial dosage administered may be increased beyond the above upper level in order to rapidly achieve the desired blood-level or tissue level, or the initial dosage may be smaller than the optimum and the daily dosage may be progressively increased during the course of treatment depending on the particular situation. If desired, the daily dose may also be divided into multiple doses for administration, for example, two to four times per day.

The pharmaceutical compositions of the disclosure may be administered in dosages sufficient to decrease the expression of LRRK2, and/or treat LRRK2-associated disease or condition, e.g., Parkinson's Disease. In therapeutic use for treating, preventing, or combating, neurodegeneration in subjects, the compounds or pharmaceutical compositions thereof will be administered orally or parenterally at a dosage to obtain and maintain a concentration, that is, an amount, or blood-level or tissue level of active component in the animal undergoing treatment which will be effective. The term “effective amount” is understood to mean that the compound of the disclosure is present in or on the recipient in an amount sufficient to elicit biological activity. Generally, an effective amount of dosage of active component will be in the range of from about 1 μg/kg to about 100 mg/kg, preferably from about 10 μg/kg to about 10 mg/kg, more preferably from about 100 μg/kg to about 1 mg/kg of body weight per day.

### V. Kits

The present invention also provides kits for treating an LRRK2-associated disorder or condition, or reducing neuronal cell death, or preventing neurodegeration or neuroinflammation in a subject in need thereof. The kits include one or more agents that decrease the expression of LRRK2, e.g., an ATIC inhibitor, an ATIC substrate (e.g., AICAR/ZMP), a precursor of ATIC substrate (e.g., AICAr), an agent that increases the expression and/or activity of AUF1, or an agent that increases the expression and/or activity of DCP1 and/or DCP2, as described herein.

The kits also include instructions for use. The instructions will generally include information about the use of the kit for editing nucleic acid molecules. In other embodiments, the instructions include at least one of the following: precautions; warnings; clinical studies; and/or references. The instructions may be printed directly on the container (when present), or as a label applied to the container, or as a separate sheet, pamphlet, card, or folder supplied in or with the container. In a further embodiment, a kit can comprise instructions in the form of a label or separate insert (package insert) for suitable operational parameters.

The kit can further contain one more additional reagent, such as an immunosuppressive reagent, a cytotoxic agent or a radiotoxic agent or one or more additional agents of the invention, as described herein. Kits typically include a label indicating the intended use of the contents of the kit. The term label includes any writing, or recorded material supplied on or with the kit, or which otherwise accompanies the kit.

In certain embodiments, the kits can also comprise, e.g., a buffering agent, a preservative, a protein stabilizing agent, reaction buffers. Each component of the kit can be enclosed within an individual container and all of the various containers can be within a single package, along with instructions for interpreting the results of the assays performed using the kit.

The kits of the invention may optionally comprise additional components useful for performing the methods of the invention. The kit can further include a delivery device, e.g., for delivery to the central nervous system, such as needles, syringes, pumps, and package inserts with instructions for use.

### Other Embodiments

All publications, patents, and patent applications mentioned in this specification are incorporated herein by reference in their entirety to the same extent as if each individual publication, patent, or patent application was specifically and individually indicated to be incorporated by reference in its entirety. Where a term in the present application is found to be defined differently in a document incorporated herein by reference, the definition provided herein is to serve as the definition for the term.

While the invention has been described in connection with specific embodiments thereof, it will be understood that invention is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

This invention is further illustrated by the following examples which should not be construed as limiting. The entire contents of all references, patents and published patent applications cited throughout this application, as well as the Figures and the Sequence Listing, are hereby incorporated herein by reference.

### EXAMPLES

### Example 1. Regulation of LRRK2 mRNA Stability by ATIC and its Substrate AICAR Through ARE-Mediated mRNA Decay

### ATIC Regulates LRRK2-Induced Toxicity and Expression Levels.

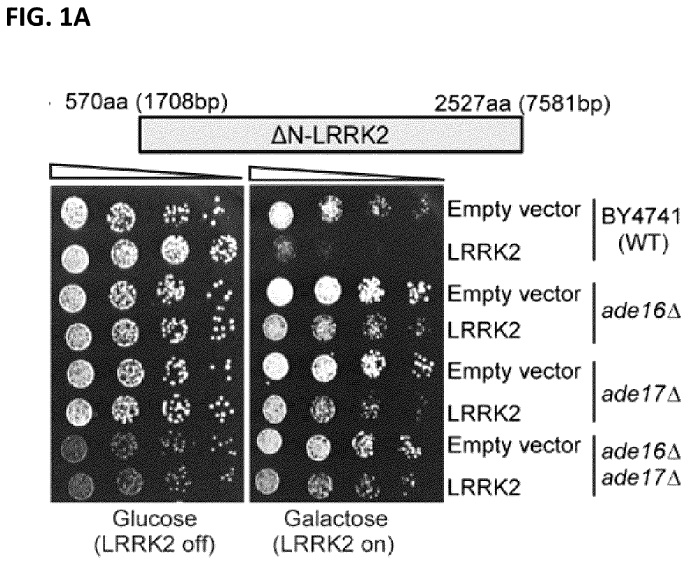

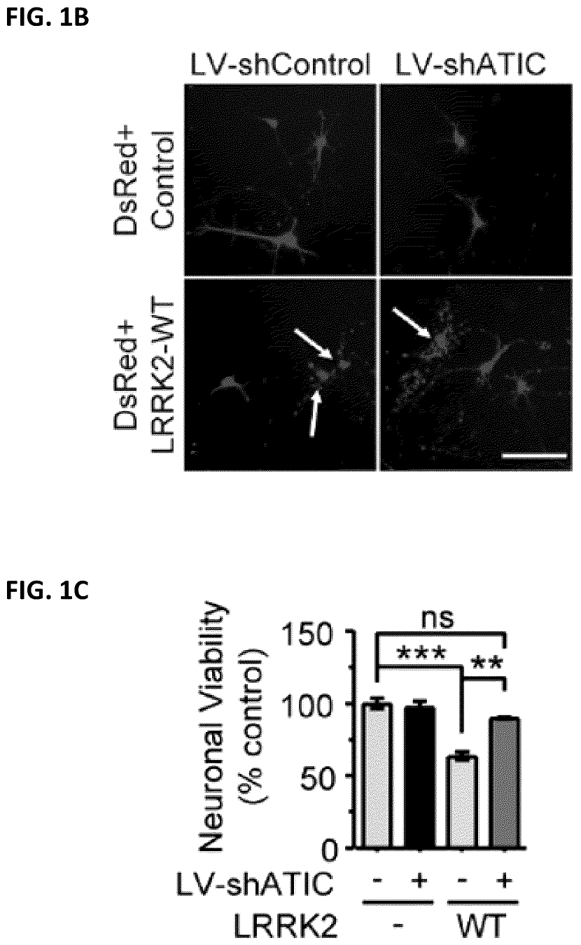

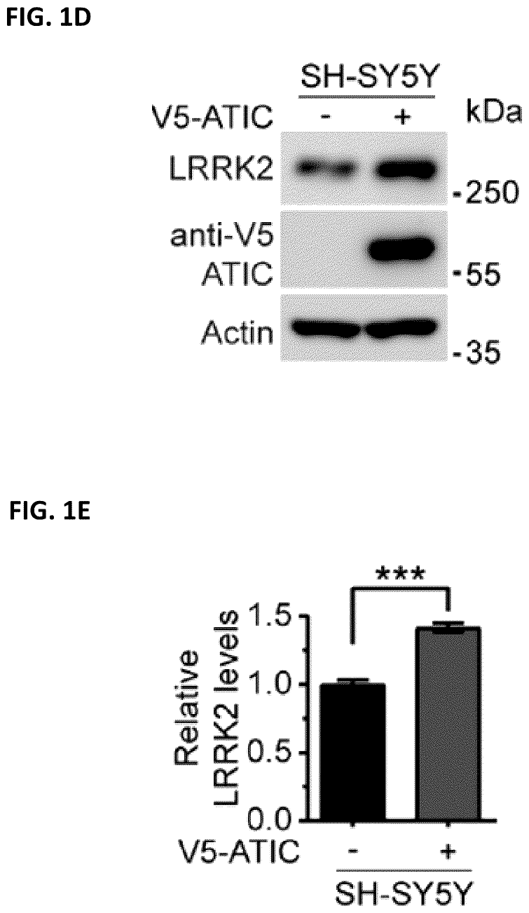

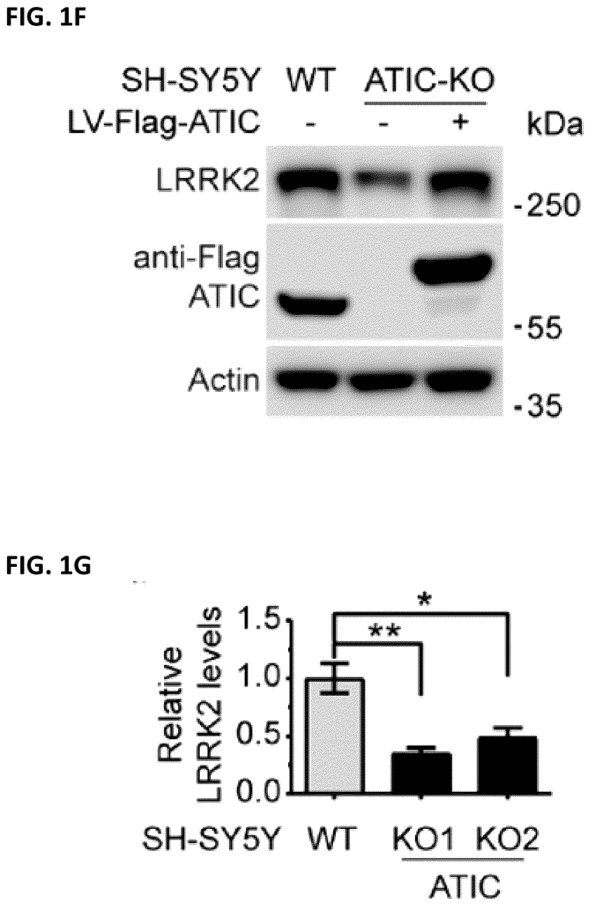

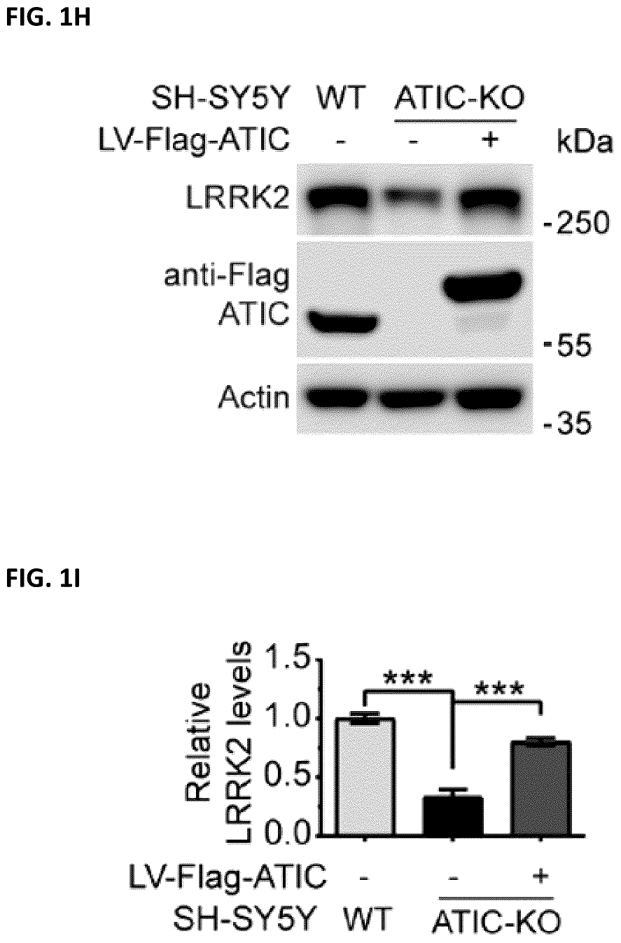

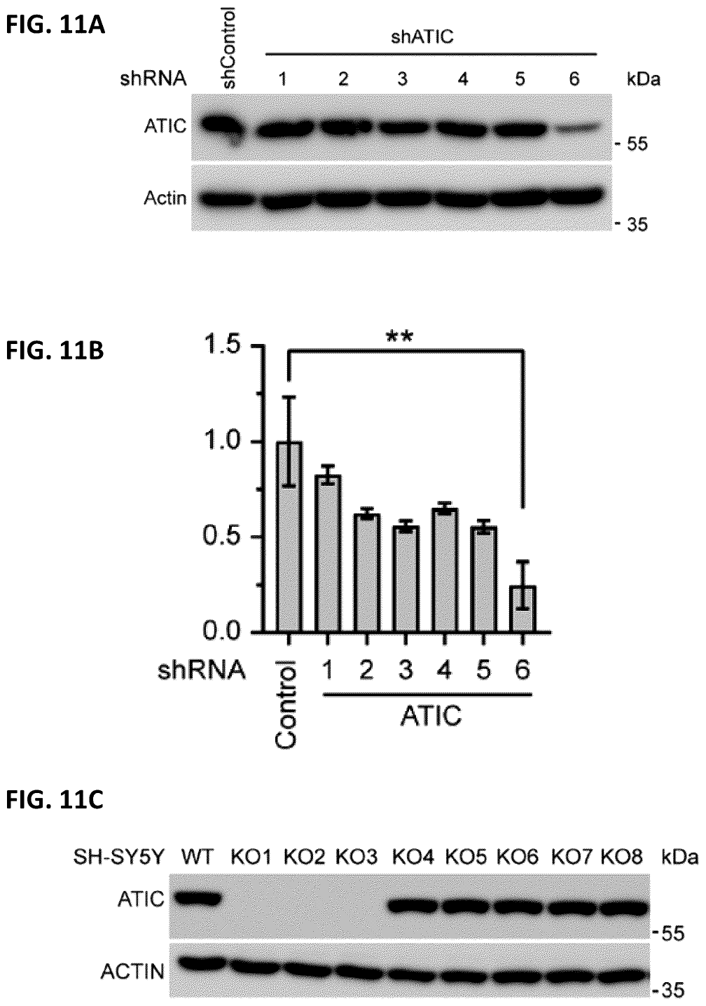

In an effort to investigate how LRRK2 mediate cell toxicity, an unbiased genome-wide genetic screen was performed using haploid-based synthetic lethality analysis with LRRK2 fragment lacking the N-terminal region (AN-LRRK2, 570-2527aa), which was adapted from previous studies^{11,30,31}. Yeast deletion mutants that suppress or enhance LRRK2-induced toxicity were identified. Deletion mutants of yeast ADE16 and ADE17 were identified to rescue LRRK2-induced toxicity that was further confirmed by cell viability assays (FIG. 1A). The human homolog of ADE16 and ADE17 is a single gene ATIC encoding a bifunctional 5-aminoimidazole-4-carboxamide ribonucleotide (AICAR)-formyltransferase/IMP cyclohydrolase, the last enzyme in the highly conserved de novo purine biosynthetic pathway, which metabolizes AICAR. As knockout of ATIC in yeast rescues LRRK2-induced toxicity, it was then explored whether knockdown of mammalian ATIC mediates LRRK2 induced neuronal toxicity (FIG. 1B, FIG. 1C). To this end, ATIC was knocked down by lenti-shRNA to ATIC (LV-shATIC) (FIG. 11A, FIG. 11B). LV-shATIC #6, which reduced ATIC levels up to ˜80%, were transduced into mouse primary neurons carrying overexpressed LRRK2 wild type (WT) co-transfected with DsRed (FIG. 1B). The effects of shATIC on neuronal viability were assessed by an established assay from our previous studies to examine neurite process length, fragmentation as indicators of neuronal viability^{6,11,14,32,33}. Indeed, similar as in yeast, shATIC rescued overexpression of LRRK2-WT induced neuronal toxicity (FIG. 1B, FIG. 1C). The underlying mechanisms of AITC mediation of LRRK2-induced neuronal toxicity were further investigated. Interestingly, it was found that overexpressing ATIC dramatically increased endogenous LRRK2 protein levels in SH-SY5Y cells (FIG. 1D, FIG. 1E). To study the effects of endogenous ATIC on LRRK2, human neuroblastoma SH-SY5Y cell lines with depletion of ATIC were generated using the CRISPR/Cas9 editing system. The resulting ATIC KO cells led to no detectable ATIC protein (FIG. 11C). It was observed that knocking out ATIC decreased LRRK2 endogenous levels (FIG. 1F, FIG. 1G). To further confirm that the reduced LRRK2 endogenous levels is specific due to ATIC knockout, a rescue experiment was performed by transfecting lentivirus carrying ATIC (LV-Flag-ATIC) back into ATIC KO cells and the resulting LRRK2 endogenous levels were examined. Indeed, it was found that transduced LV-ATIC rescued LRRK2 endogenous levels to a level closed to that in WT cells (FIG. 1H, FIG. 1I), suggesting the specificity of ATIC on LRRK2 endogenous levels. Taken together, ATIC regulates LRRK2-induced toxicity and expression levels.

### AICAr, the Precursor of ATIC Substrate, Regulates LRRK2 Expression Levels and Neuronal Toxicity.

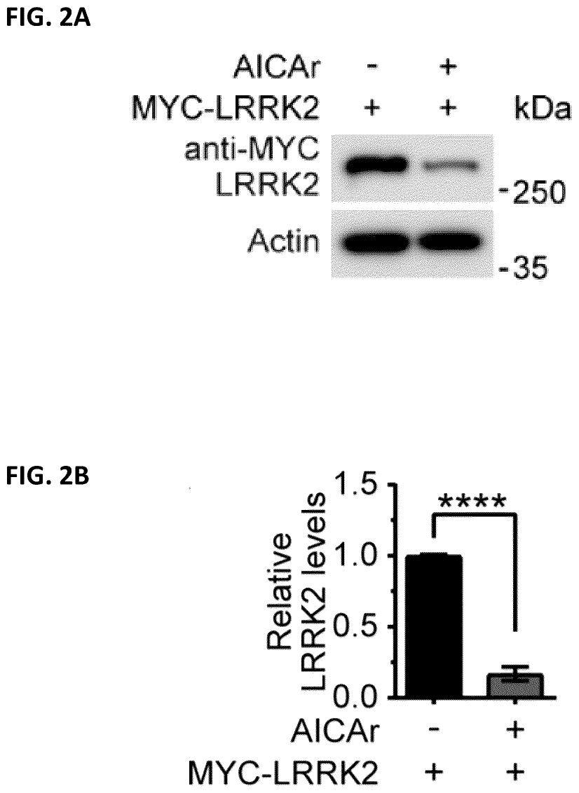

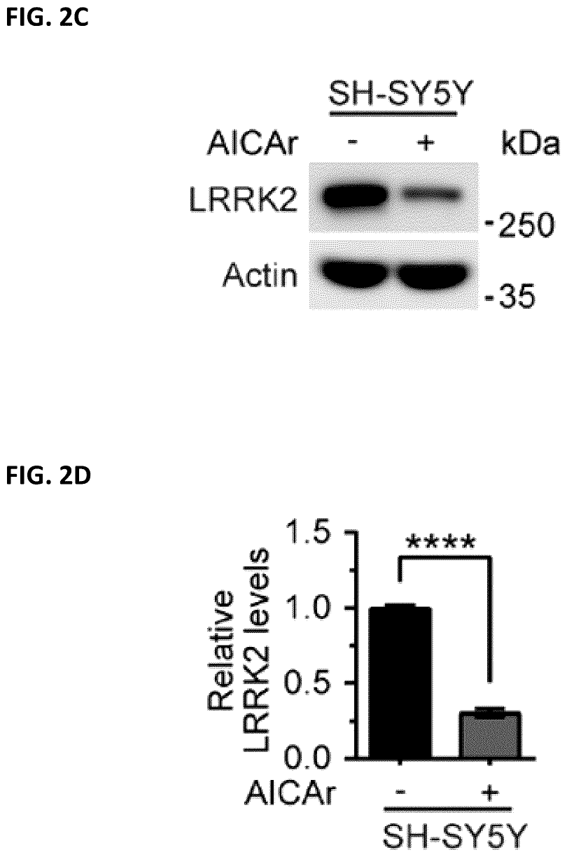

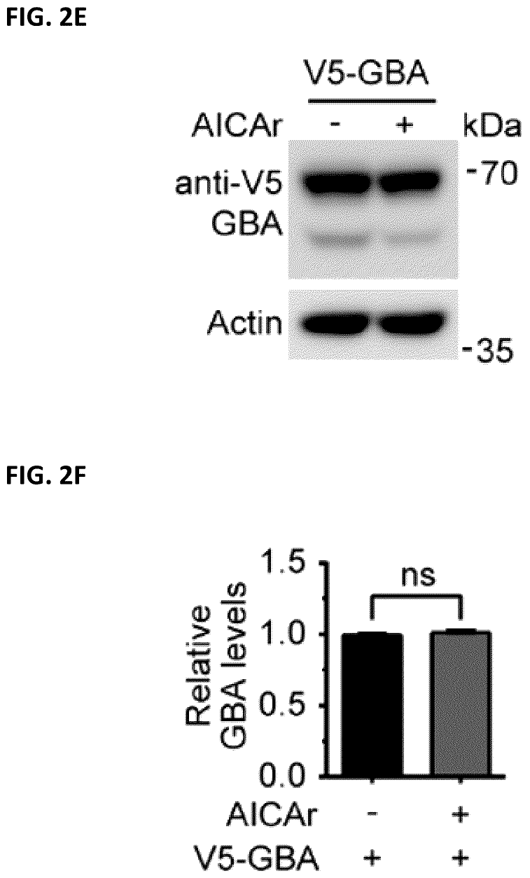

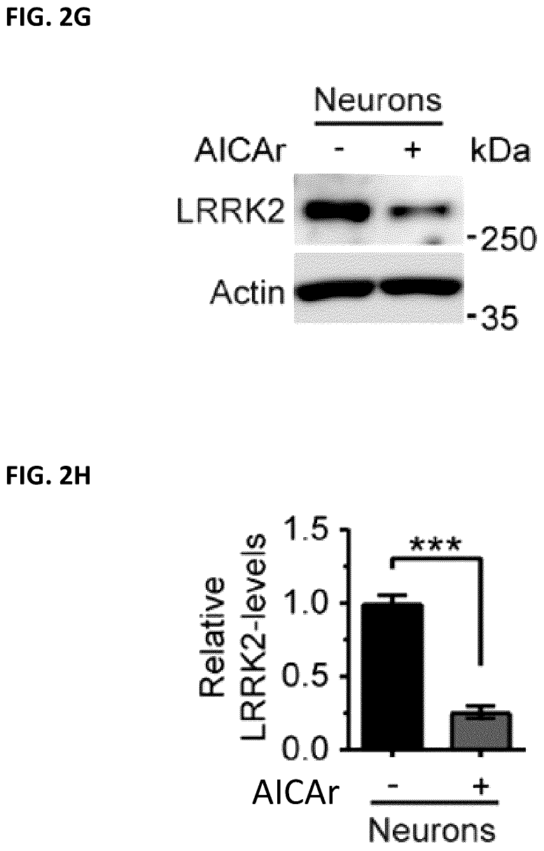

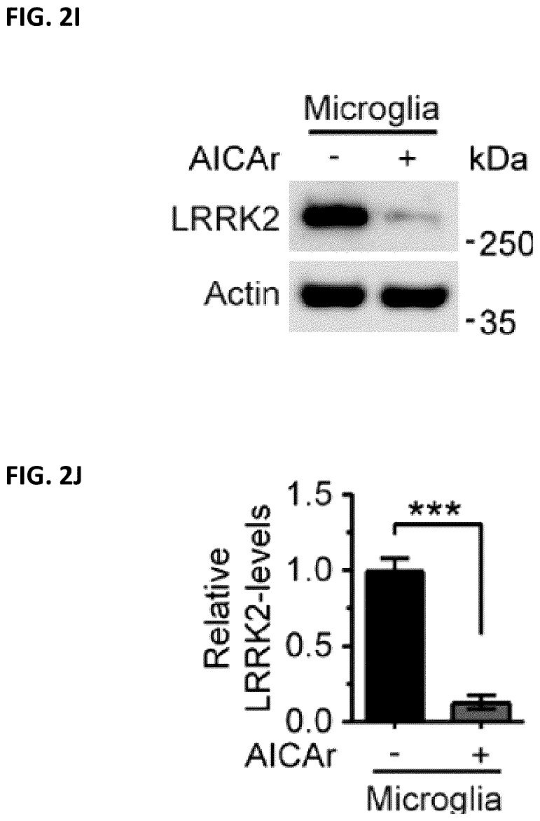

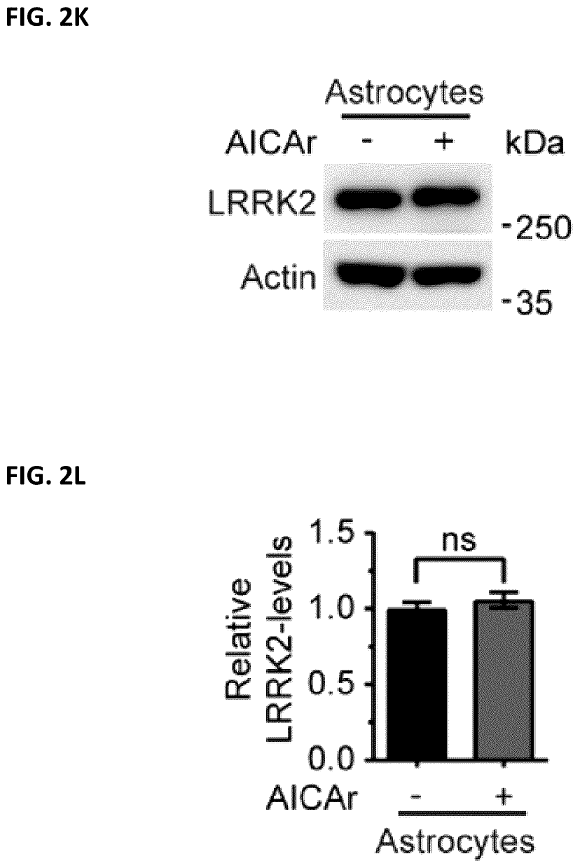

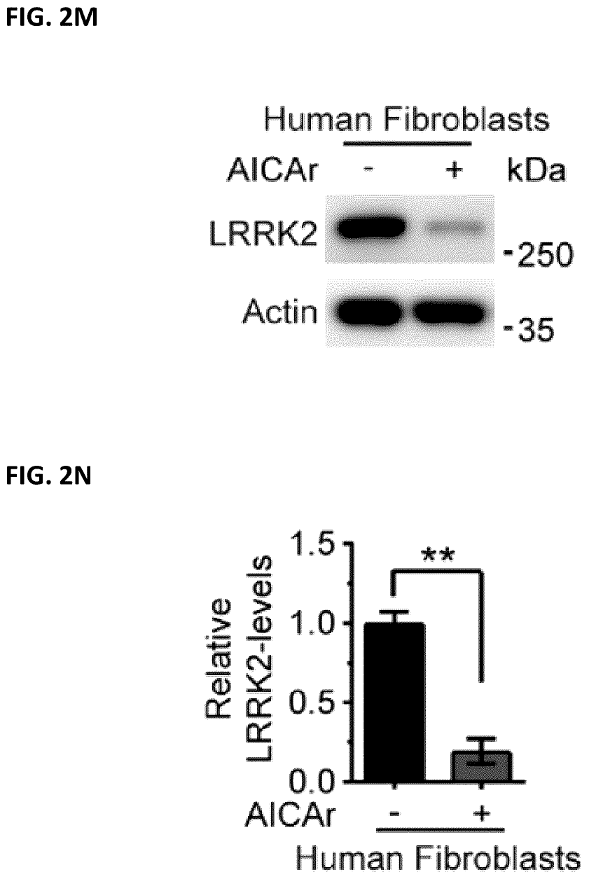

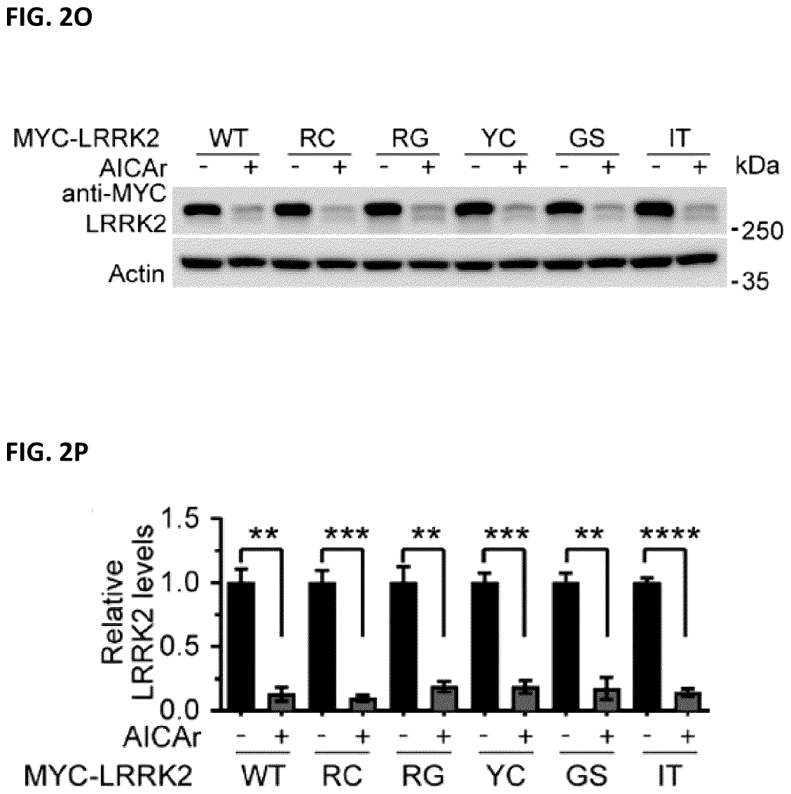

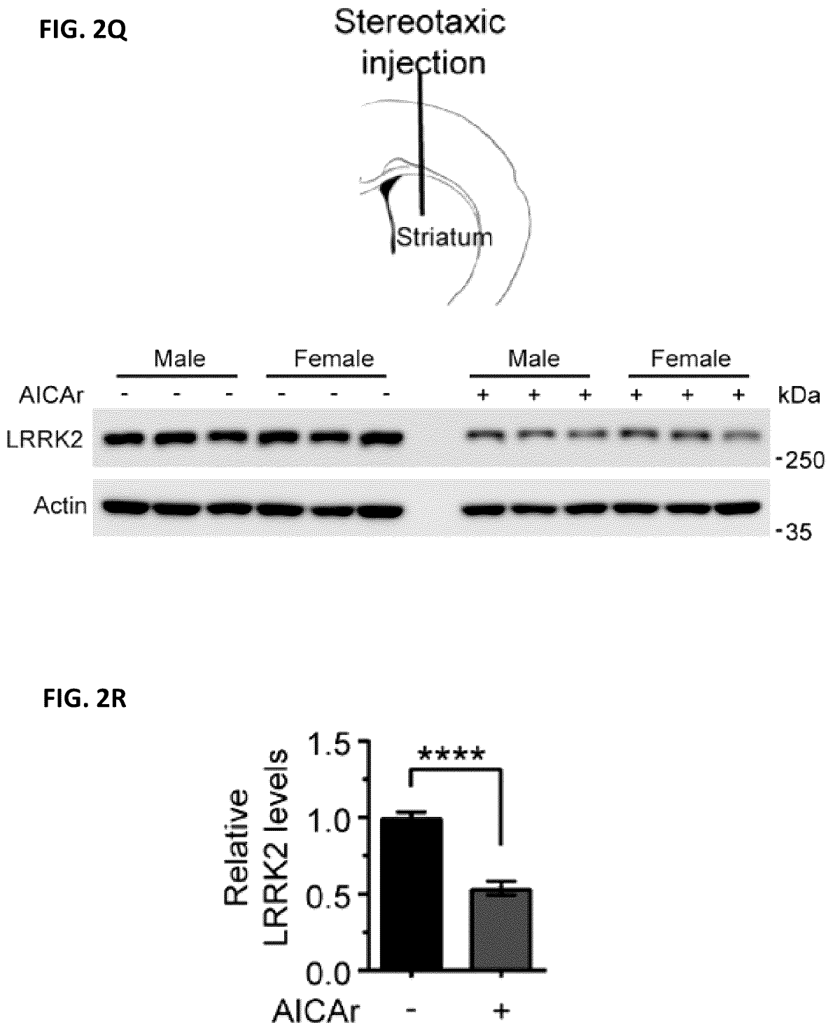

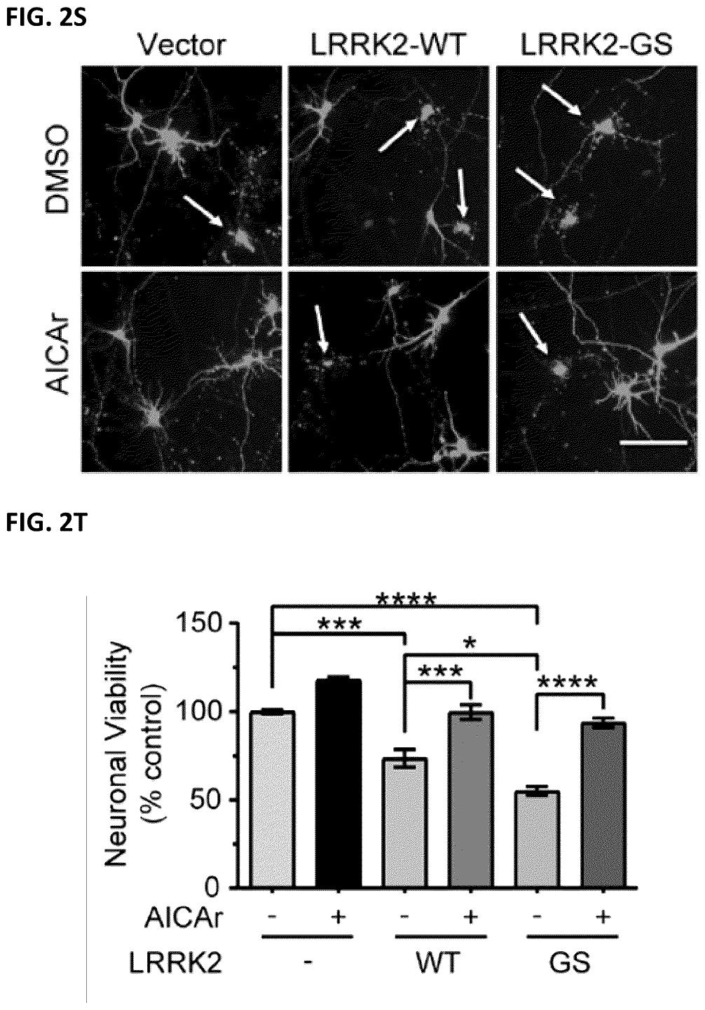

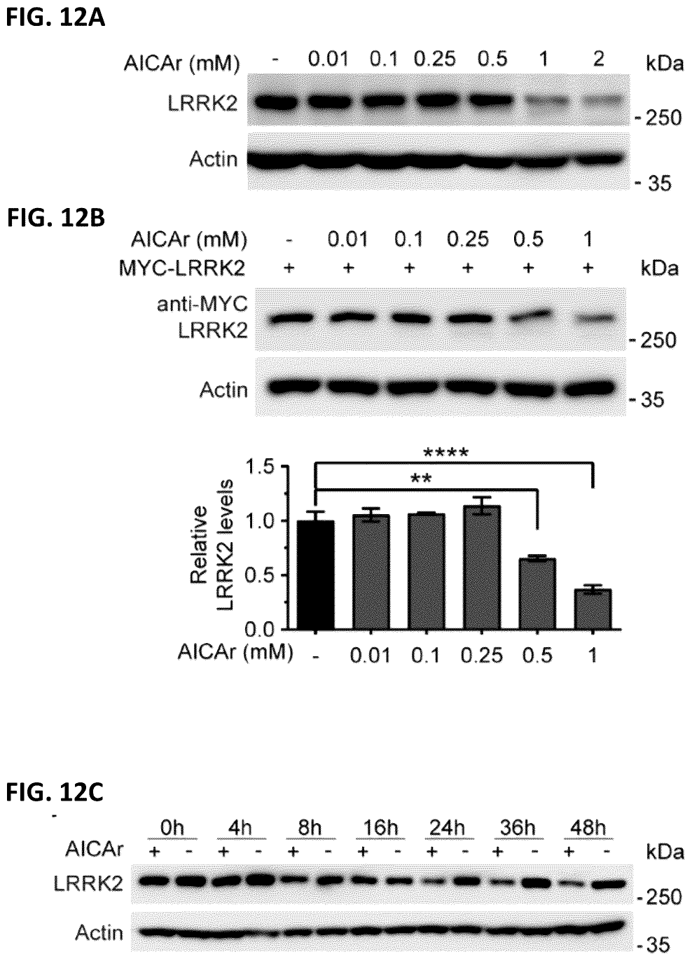

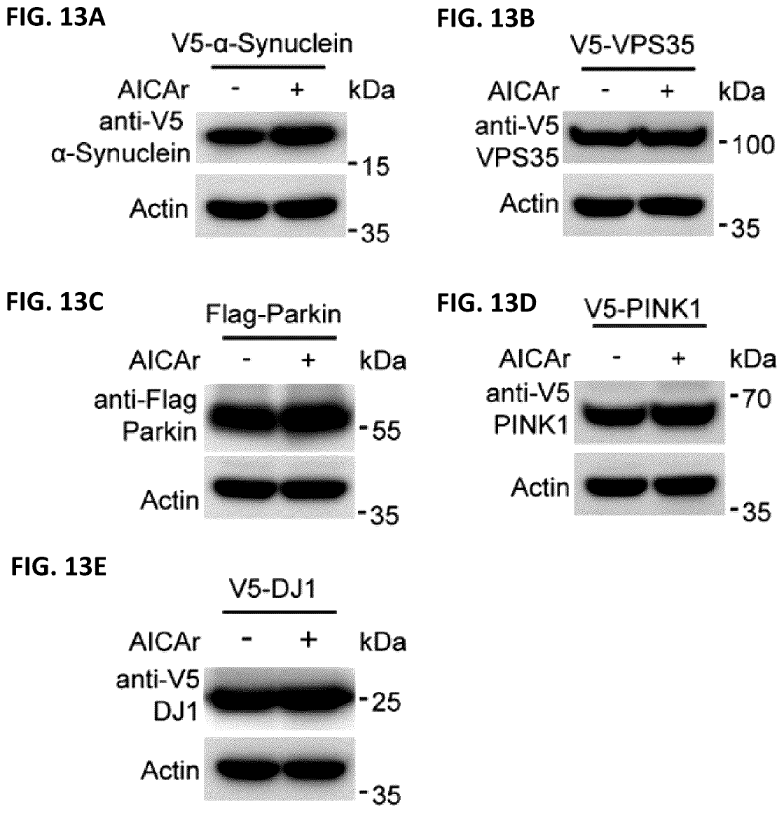

As ATIC metabolizes its substrate AICAR, also called ZMP, the knockdown or knockout of ATIC would accumulate AICAR/ZMP. Indeed, it was found that AICAR/ZMP was increased significantly in ATIC KO cells at a concentration of 1557 ng/ml while it was undetectable in WT cells using UPLC (Ultra-Performance Liquid Chromatography) (Table 1). The effect of AICAR treatment on LRRK2 expression levels and LRRK2-induced neuronal toxicity was evaluated. As AICAR/ZMP is membrane impermeable, the cells were treated with the cell-permeable AICAR/ZMP precursor AICA riboside (AICAr), a non-monophosphate form of AICAR/ZMP, which can be transported into cells through adenosine transporter (AT) and then converted into 5′-monophosphate AICAR/ZMP by adenosine kinase (ADK). SH-SY5Y cells were used to study endogenous LRRK2 as this cell line expresses relatively high levels of LRRK2 while HEK293 cells were used to study overexpressed LRRK2 as this cell line has high transfection efficiency with undetectable LRRK2 endogenous levels. Indeed, AICAr decreased overexpressed LRRK2 levels in HEK293 cells (FIG. 2A, FIG. 2B), and endogenous LRRK2 levels in SH-SY5Y cells (FIG. 2C, FIG. 2D), similar to that of ATIC knockout. The AICAr treatment time and concentration were optimized on both overexpressed (FIG. 12A) and endogenous LRRK2 level (FIG. 12B). 1 mM AICAr with 24h-48h treatment suppressed LRRK2 level dramatically (FIG. 12C). Notably, this effect is LRRK2 specific, as no changes of other PD-associated proteins such as GBA (glucocerebrosidase; FIG. 2E, FIG. 2F), α-Synuclein, VPS35, Parkin, PINK, and DJ-1 (FIG. 13) upon the same treatment. To further test whether AICAr regulation of LRRK2 levels is cell-type specific, the primary neurons (FIG. 2G, FIG. 2H), microglia (FIG. 2I, FIG. 2J), astrocytes (FIG. 2K, FIG. 2L) and human fibroblasts (FIG. 2M, FIG. 2N) were treated with AICAr. It was observed that AICAr can significantly decrease LRRK2 levels in neurons, microglia and patient fibroblasts. Interestingly, LRRK2 levels remained unchanged in astrocytes, suggesting that AICAr regulation of LRRK2 levels was cell-type specific. The differential effects of AICAr on the levels of LRRK2 familial mutant forms R1441C (RC), R1441G (RG), G2019S (GS), or I2020T (IT) were tested. It was found that AICAr treatment had similar effects on the levels of LRRK2 familial mutant forms as that of LRRK2 wild type (WT) form (FIG. 2O, FIG. 2P), suggesting the effect of AICAr on LRRK2 levels was independent of LRRK2 mutations.

TABLE 1
AICAR/ZMP in WT and ATIC knockout cells measured by UPLC.

| AICAR/ZMP | WT-1 | WT-2 | WT-3 | KO-1 | KO-2 | KO-3 |
|---|---|---|---|---|---|---|
| Area | n/a | n/a | n/a | 2313 | 2297 | 2155 |
| ng/mL | n/a | n/a | n/a | 1595.86 | 1585.20 | 1490.57 |
| Average | | — | | | 1557.21 | |
| Stdev | | — | | | 57.96 | |

To study the effect of AICAr on LRRK2 levels in vivo, AICAr was stereologically injected into the striatum of both male and female mice via osmotic minipumps by subcutaneous implantation (72 ug/animal/day, Alzet), as AICAr has a low permeability across the blood-brain barrier. Saline injections were performed as controls. 1 day after AICAr injection, LRRK2 levels in striatal tissue near the injection sites were dramatically decreased in both male and female mice, as analyzed by western blots (WB) (FIG. 2Q, FIG. 2R). These data suggest AICAr decreased LRRK2 levels in vivo in mouse tissues.

To examine whether AICAr treatment modulates LRRK2-induced neuronal toxicity, mouse primary neurons, which were transfected with LRRK2, were treated with 1 mM AICAr. Strikingly, AICAr significantly rescued overexpression of LRRK2-WT and GS mutant induced neuronal toxicity (FIG. 2S, FIG. 2T). This data suggests AICAr treatment rescues LRRK2 induced neuronal toxicity. Together, these data suggest that AICAr regulates LRRK2 expression levels and neuronal toxicity.

AICAr Regulates LRRK2 at mRNA Level.

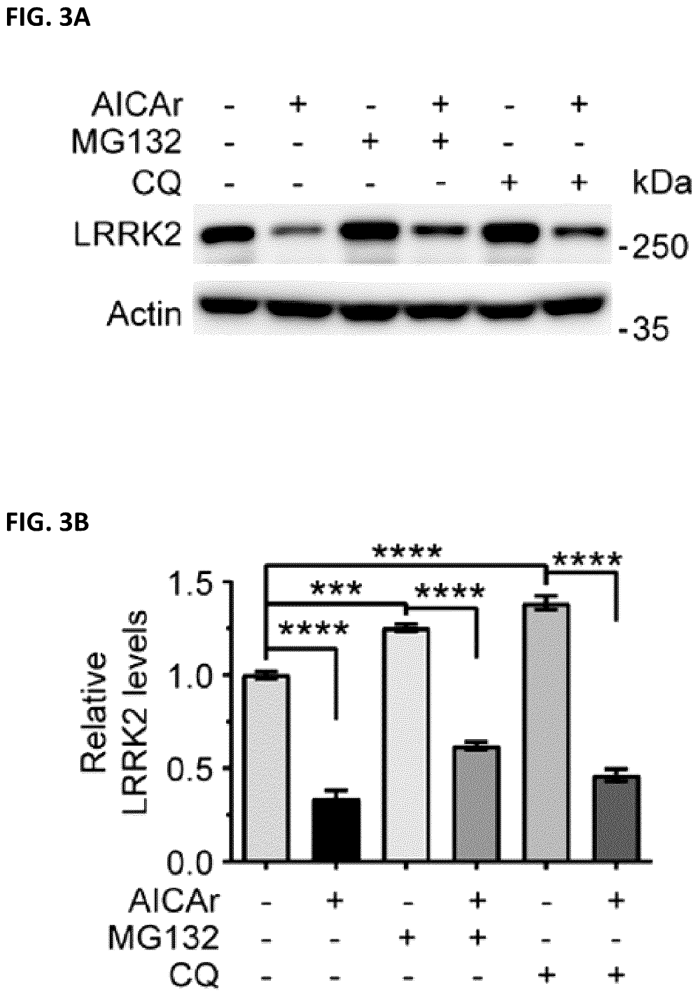

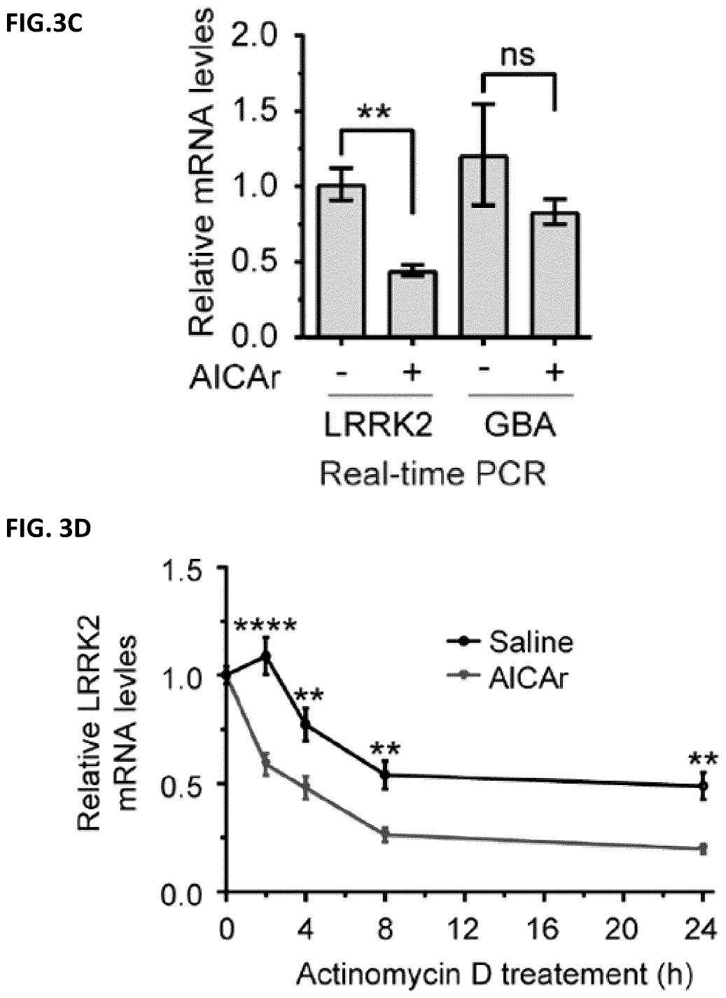

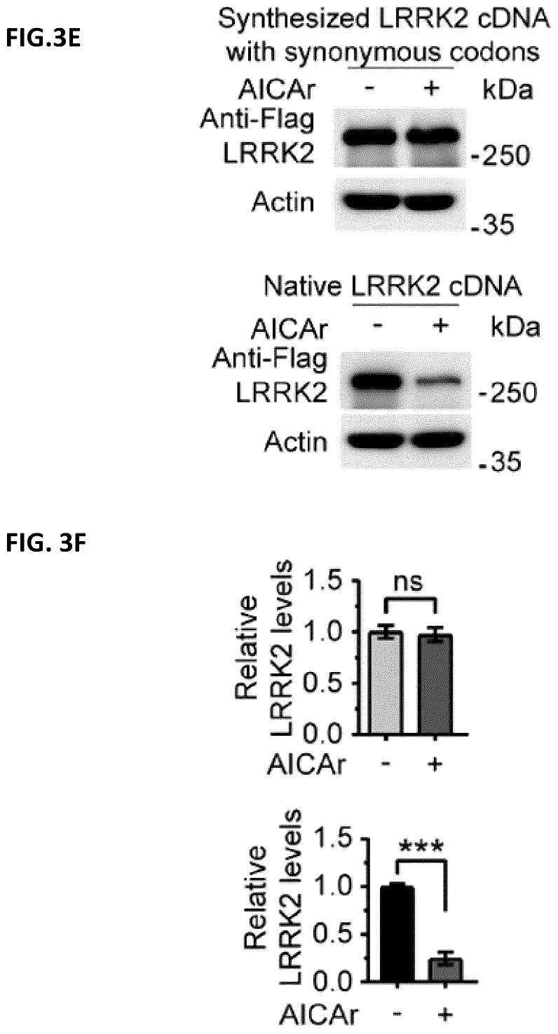

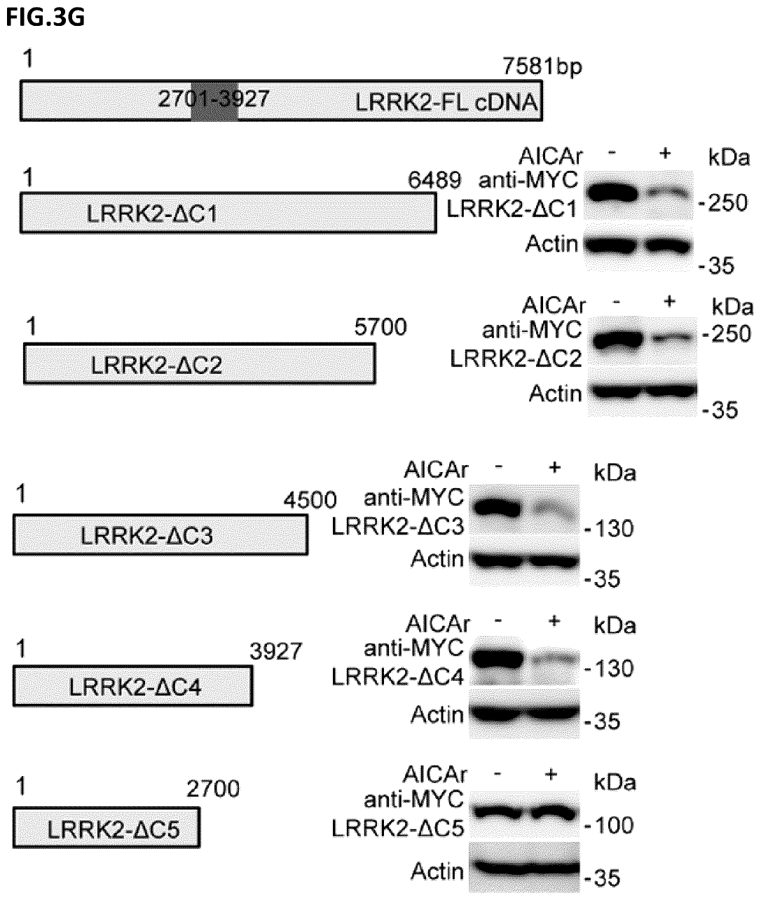

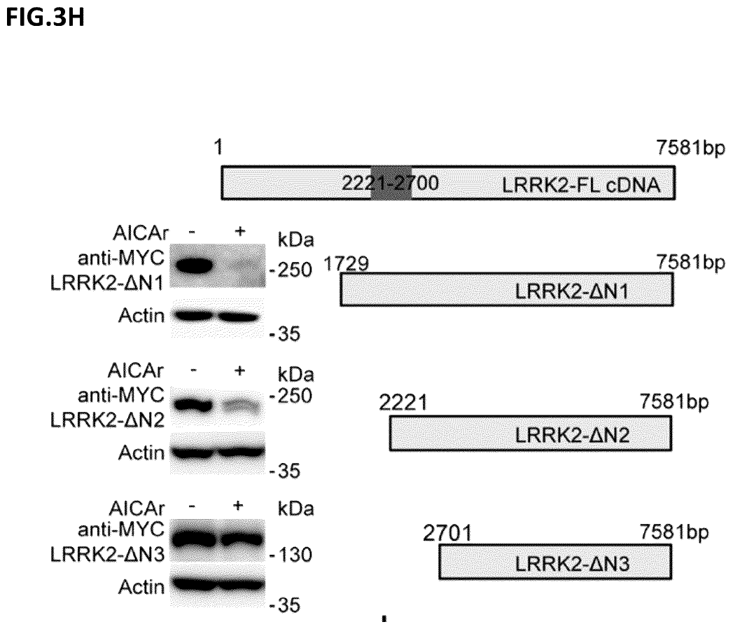

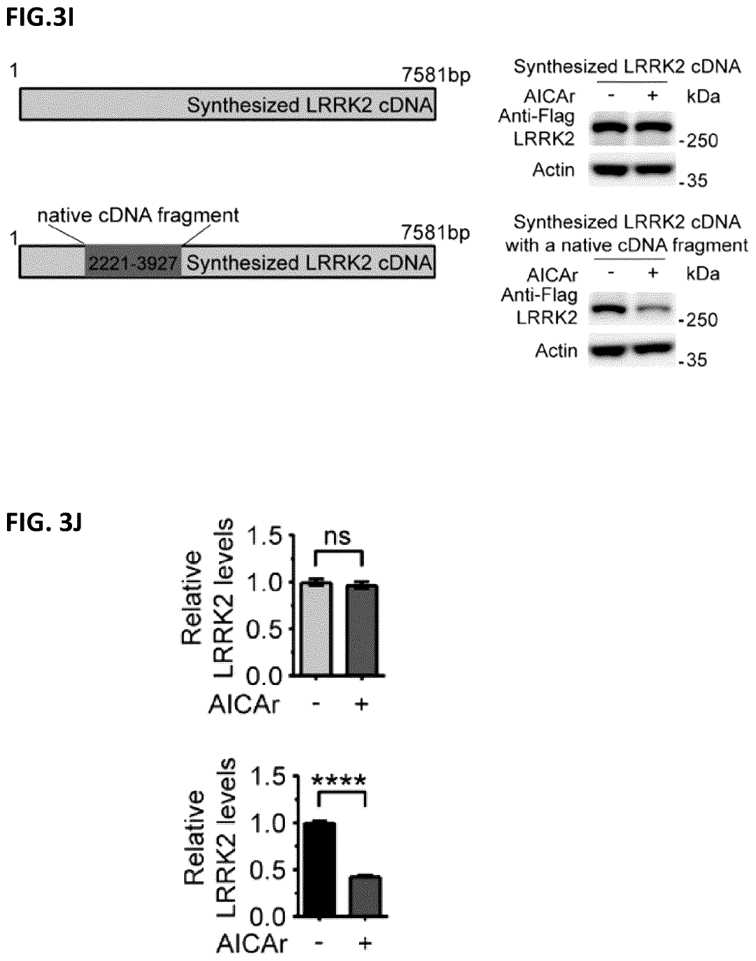

To determine the molecular mechanism of how AICAr regulates LRRK2 protein levels, it was explored whether AICAr regulates LRRK2 protein stability or LRRK2 mRNA level. First, protein stability was examined by treating the cells with MG132, a protease inhibitor, or chloroquine (CQ), an autophagy inhibitor. Despite the treatment of MG132 or CQ, AICAr still largely reduced LRRK2 protein levels, suggesting that AICAr does not regulate LRRK2 levels through protein stability (FIG. 3A, FIG. 3B). Next, LRRK2 mRNA levels were examined upon AICAr treatment by real-time PCR (FIG. 3C). Interestingly, LRRK2 mRNA levels significantly decreased upon AICAr treatment while no changes were found for the mRNA levels of GBA, another PD risk factor used as a control (FIG. 3C). Actin was used as an internal input. This suggests that AICAr specifically regulates LRRK2 at mRNA level. Since both GBA and LRRK2 were constructed in the same expression vector, the selective effect of AICAr on LRRK2 mRNA level suggests that transcriptional regulation is unlikely the cause. Another important regulatory mechanism of mRNA level is mRNA stability. Therefore, mRNA stability assay was further performed in vitro by measuring LRRK2 mRNA level over a 24-h period treatment of AICAr and in the presence of actinomycin D to block de nove transcription (FIG. 3D). LRRK2 mRNA decay was accelerated by AICAr treatment by about twofold (FIG. 3D), which suggests that AICAr mediates LRRK2 mRNA stability. As mRNA stability is largely depended on mRNA nucleotide sequence, tt was therefore reasoned whether LRRK2 nucleotide sequence plays a role in AICAr mediated LRRK2 protein levels. To this end, the same expression construct for a synthesized LRRK2 cDNA was generated with synonymous codons, of which the same LRRK2 protein will be expressed but the nucleotide sequence encoding each amino acid differs. Strikingly, in contrast to a great suppress on native LRRK2 cDNA expression, treatment of AICAr had no obvious effects on the expression of the synthesized LRRK2 cDNA with synonymous codons (FIG. 3E, FIG. 3F). Since the only difference between these two expression constructs is the LRRK2 nucleotide sequence, it was concluded that the effect of AICAr on LRRK2 protein levels depends on the nucleotide sequence of LRRK2 cDNA. Taken together, this data suggests that AICAr post-transcriptionally regulates LRRK2 mRNA levels and this regulation is sensitive to LRRK2 nucleotide sequence.

To further identify the specific region of LRRK2 cDNA that responds to AICAr treatment, a series of LRRK2 cDNA deletion mutations from N- and C-terminals were generated as illustrated in FIG. 3G and FIG. 3H. The deletion mutants were subjected to AICAr treatment. The truncated LRRK2 protein levels were determined (FIG. 3G, FIG. 3H). A region of 2221-3927 bp of LRRK2 cDNA was narrowed down to as the AICAr response element. To test whether this specific region is essential for AICAr effects on LRRK2, this region of the synthesized LRRK2 cDNA was placed with the native sequence and the response of this chimeric LRRK2 cDNA to AICAr treatment was determined. Strikingly, AICAr had a great suppression on this chimeric LRRK2 cDNA expression (FIG. 3I, FIG. 3J). Taken together, AICAr regulates LRRK2 at mRNA level and the region of 2,221-3,927 bp of LRRK2 cDNA is an AICAr response element.

### AUF1 as a Specific LRRK2 RNA Binding Protein Plays a Critical Role in AICAr-Mediated LRRK2 Levels

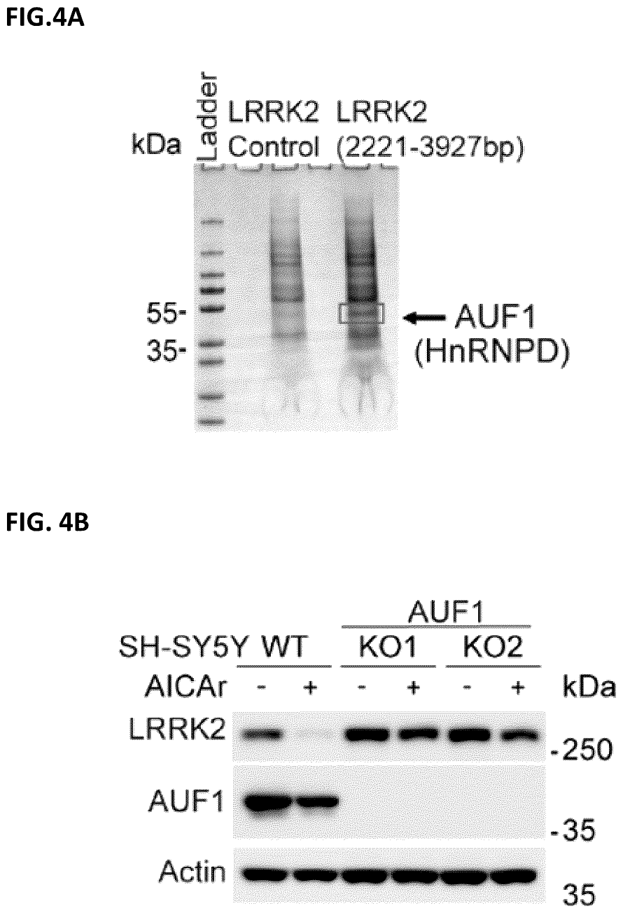

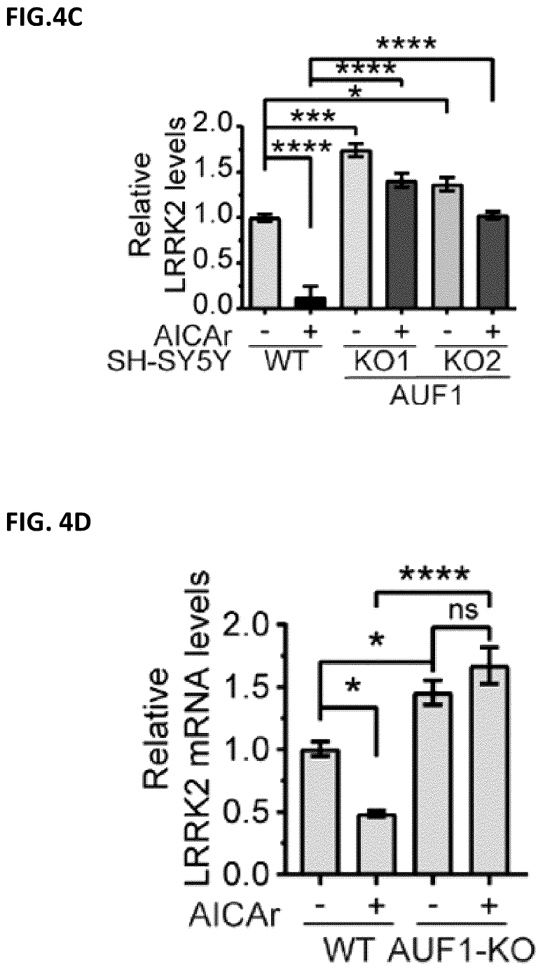

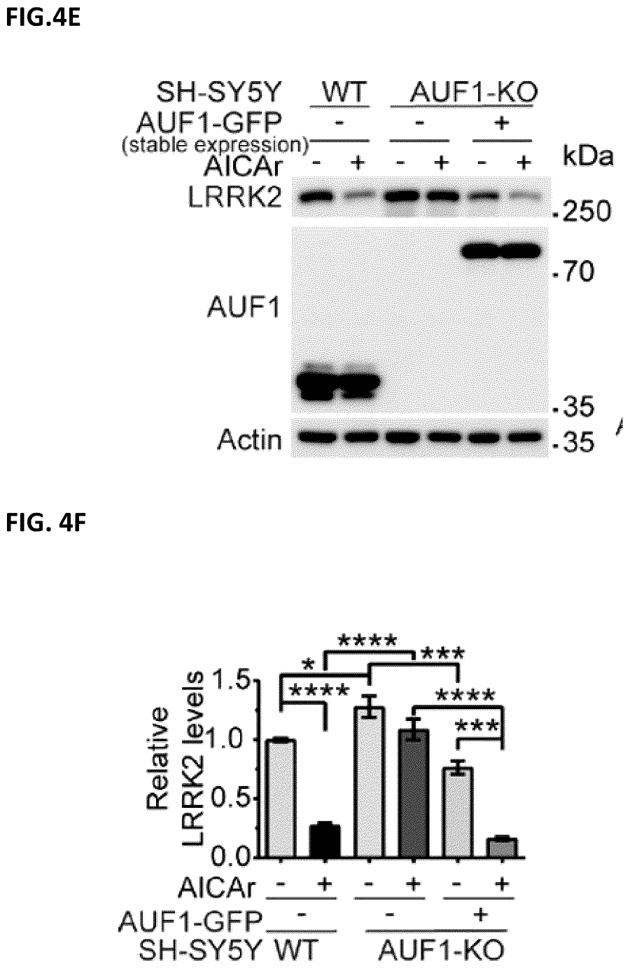

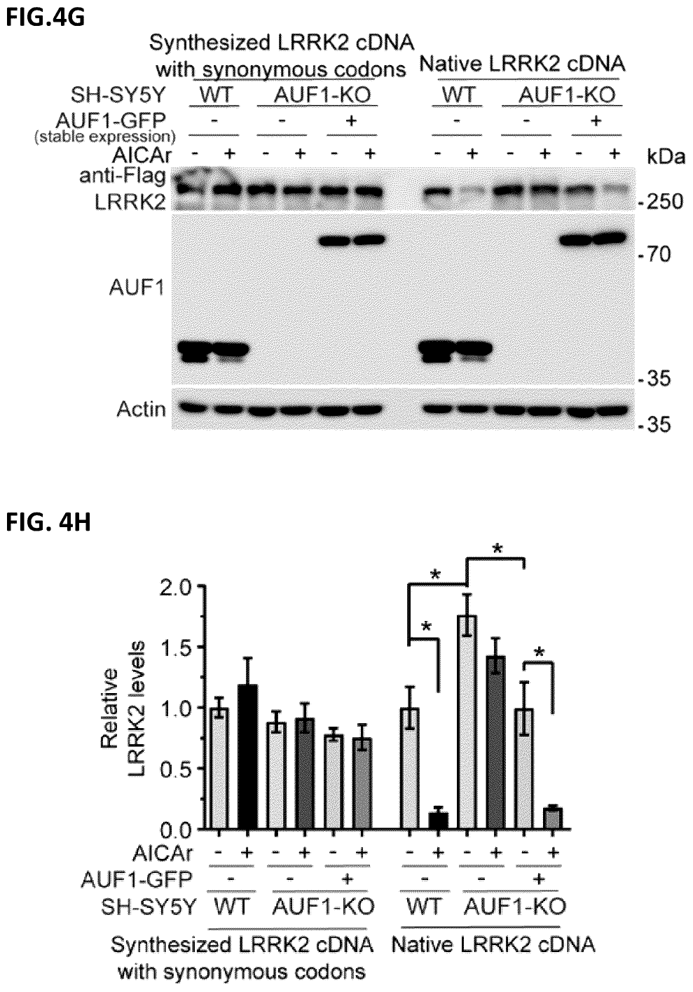

Having identified an AICAr response element of LRRK2, it was hypothesized that specific RNA binding protein(s) (RBP) may interact with this AICAr response element and mediate AICAr induced LRRK2 protein suppression. An RNA-pull-down assay was performed to identify potential RBPs using the identified AICAr response element of LRRK2 as a bait (FIG. 4A). An LRRK2 cDNA region outside of the AICAr response element was used as a negative control. After a series wash and elution, the pull-down samples were subjected to mass spectrometry (MS) analysis. Notably, the AU-rich element (ARE)/poly(U)-binding/degreadation factor 1 (AUF1)/hnRNP D was on the top of the list from our MS analysis. AUF1 is known to bind to ARE-mRNAs and regulates subsequent stability, translation or subcellular localization of the target mRNA³⁴. Further analyses using the RBPmap³⁵ predicted multiple AUF1 binding motifs in native LRRK2 cDNA but not synthesized LRRK2 cDNA. These results therefore suggest that AICAr may regulate LRRK2 levels through ARE-mediated mRNA decay (AMD).

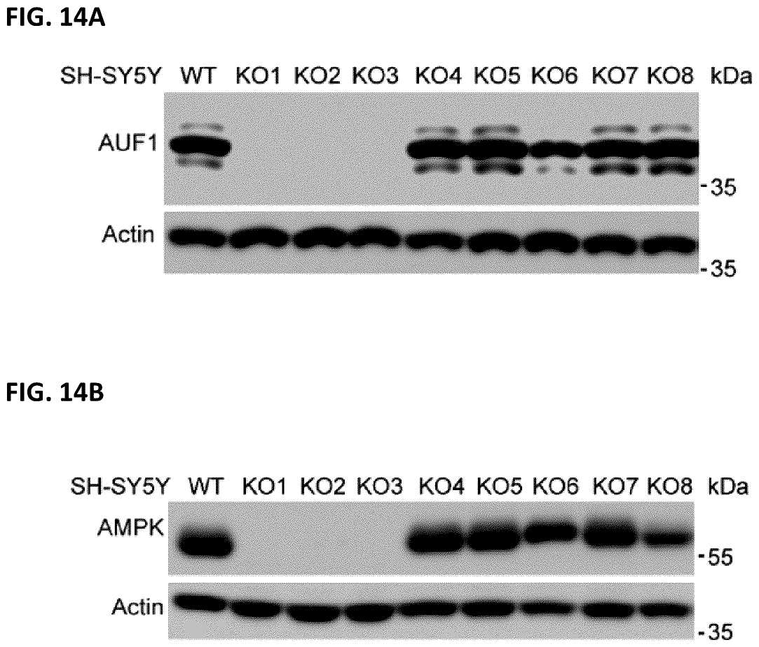

To further investigate whether AUF1 is involved in AICAr-regulated LRRK2 protein levels, the effects of endogenous AUF1 on LRRK2 levels were examined. Neuroblastoma SH-SY5Y cell lines with depletion of AUF1 were generated using the CRISPR/Cas9 editing system. The resulted AUF1 KO cells led to no detectable AUF1 protein (FIG. 14A). Notably, knockout of AUF1 increased LRRK2 protein levels (FIG. 4B, FIG. 4C) and mRNA levels (FIG. 4D). Interestingly, minimal effect of AICAr on LRRK2 mRNA level was found in AUF1-KO cells (FIG. 4D), and largely attenuated effects of AICAr on LRRK2 protein levels in AUF1-KO cells were found (FIG. 4B, FIG. 4C, FIG. 4E, FIG. 4F, FIG. 4G, and FIG. 4H). This suggests that AICAr decreases LRRK2 level mainly through AUF1. To further confirm that the reduced effect of AICAr on LRRK2 is specific due to AUF1 knockout, a rescue experiment was performed by transfecting AUF1 back into AUF1 KO cells and the resulting AICAr effects on LRRK2 levels were examined. To achieve the maximum rescue results, AUF1-GFP stable expression was generated in the AUF1 KO cells. Indeed, it was found that stable expression of AUF1 significantly rescued AICAr effects on LRRK2 in SH-SY5Y cells (FIG. 4E, FIG. 4F, FIG. 4G, and FIG. 4H). In contrast to LRRK2 expression from native LRRK2 cDNA, AUF1 did not regulate LRRK2 expression from synthesized LRRK2 cDNA with synonymous codons (FIGS. 4G, FIG. 4H), indicating that AUF1-mediated LRRK2 regulation also depends on LRRK2 nucleotide sequence as AICAr does. Therefore, it was concluded that AUF1 plays a critical role in AICAr-regulated LRRK2 levels.

AUF1 Interacts with LRRK2 mRNA.

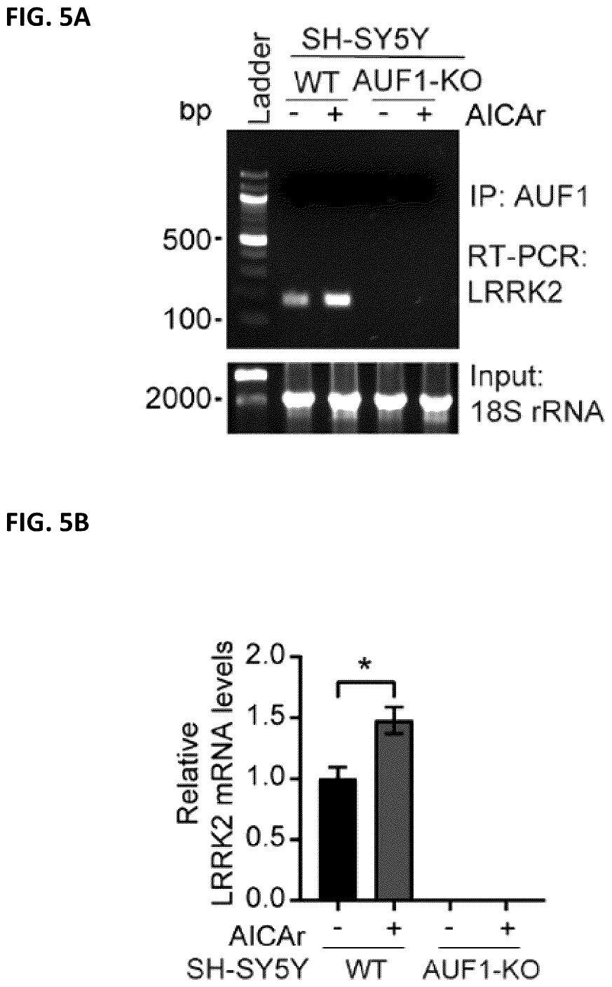

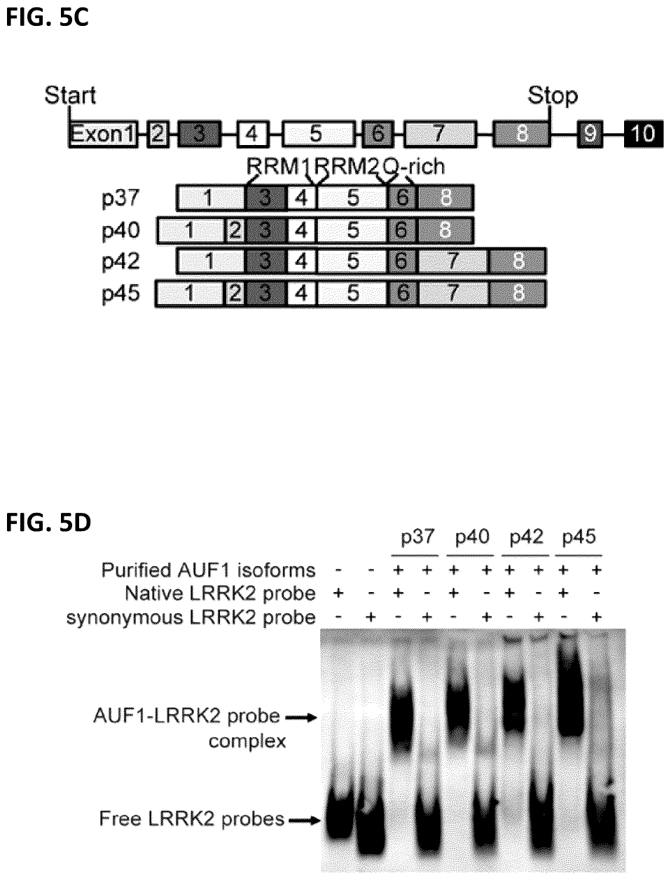

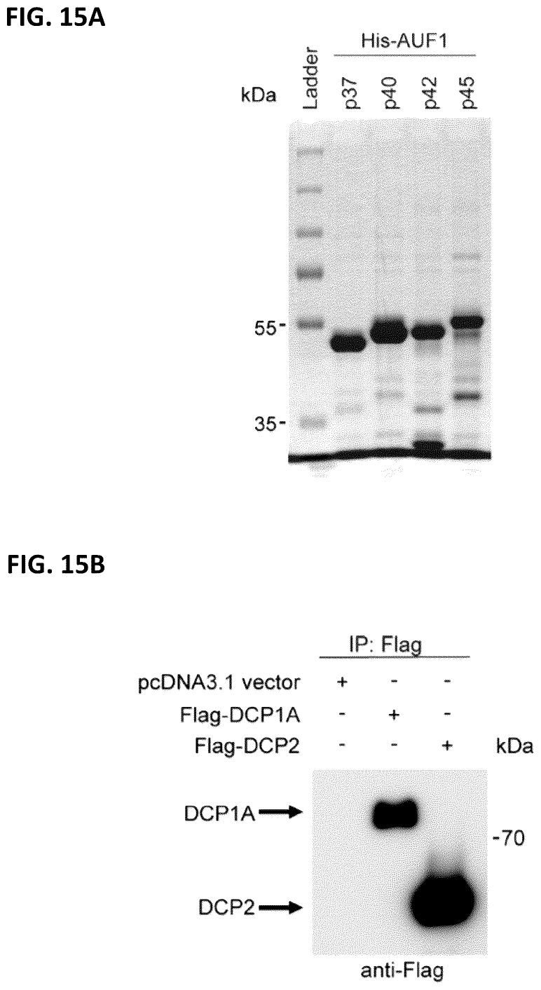

As knockout of AUF1 attenuated the effect of AICAr on LRRK2 levels, it was hypothesized that an AICAr-AICAR-AUF1-LRRK2 axis regulates LRRK2 levels. The interaction between AUF1 and LRRK2 mRNA was first examined. To ask whether AUF1 interacts with LRRK2 mRNA in cells, RNA immunoprecipitation (RIP) assay was performed in cells at endogenous levels and it was found that LRRK2 was significantly enriched in the AUF1 antibody-RIP complex compared with AUF1 knockout cells (FIGS. 5A, 5B), suggesting that endogenous LRRK2 mRNA binds to AUF1 in cells. Further, an RNA electrophoretic mobility shift assay (EMSA) assay was performed to examine whether LRRK2 mRNA directly binds to four AUF1 protein isoforms in vitro (FIGS. 5C, 5D). AUF1 protein has four isoforms: p37 (37 kD), p40 (40 kD), p42 (42 kD), p47 (47 kD) (FIG. 5C). Each isoform is a product from alternative mRNA splicing. Four recombinant AUF1 isoforms were purified from bacteria (FIG. 15A). A 62-nt Native LRRK2 mRNA probe which has enriched ARE elements in the AUF1-LRRK2 specific binding region ((2221-3927 bp) was employed with four purified AUF1 isoforms in the EMSA assay. Indeed, the native LRRK2 mRNA probe directly bound to four AUF1 protein isoforms (FIG. 5D). The 62-nt LRRK2 probe from synthesized LRRK2 in the same region was utilized as a control. Interestingly, the LRRK2 probe from synthesized LRRK2 could not bind to any of AUF1 protein isoforms (FIG. 5D). These results suggest specific binding between native LRRK2 mRNA and AUF1.

The Decapping Enzyme Complex DCP1/2 is Involved in LRRK2 mRNA Delay

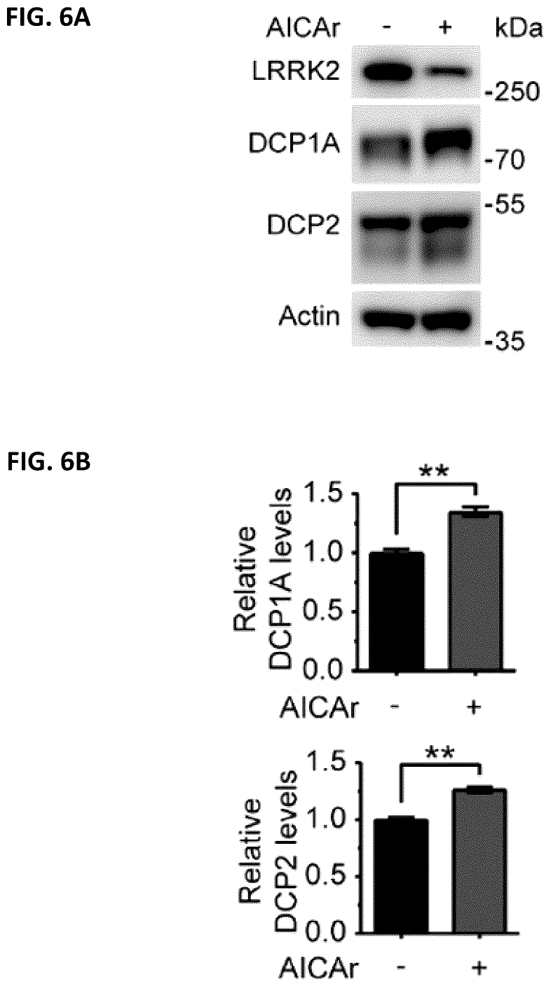

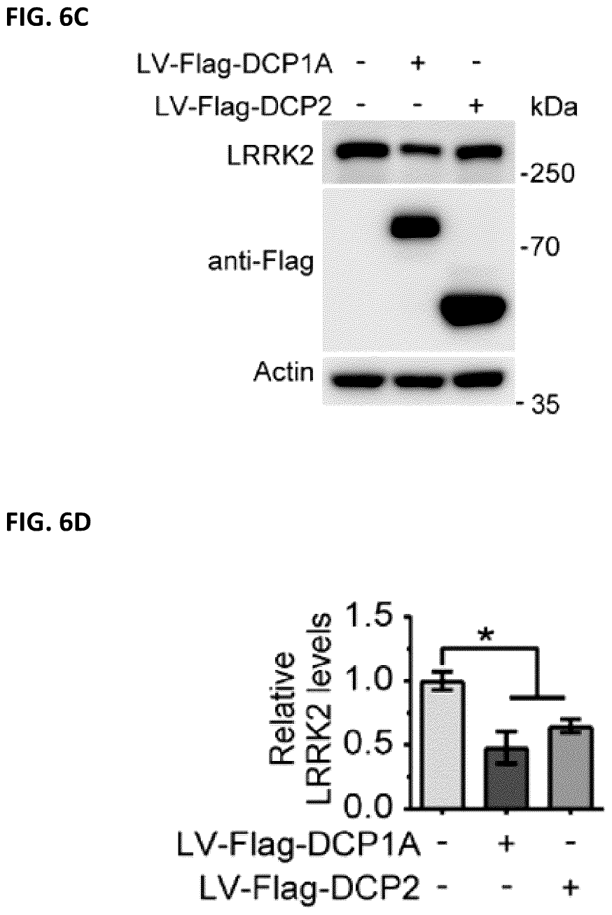

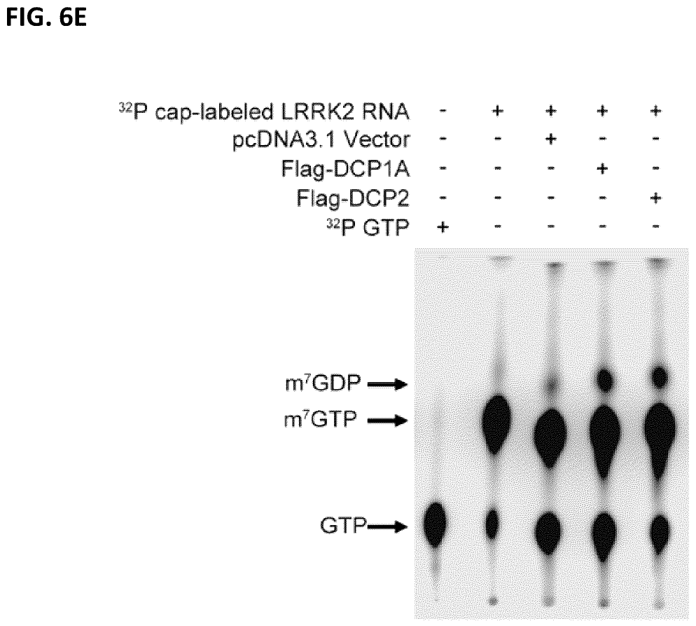

It has been proposed that AUF1 binds to AREs of ARE-rich mRNAs and recruits the decapping enzymes DCP1/2 and other decapping proteins, leading to removal of the 5′ cap of mRNA and therefore mRNA decay.^{34,36,37}. Since it was demonstrated that AUF1 is critical for AICAr regulated LRRK2 levels and AUF1 binds to LRRK2 mRNA directly, it was asked whether the decapping complex is recruited and decaps LRRK2 mRNA. To test this possibility, the endogenous levels of the DCP1A and DCP2 were first examined upon AICAR treatment. Indeed, AICAr treatment significantly increased the endogenous levels of both DCP1A and DCP2 (FIGS. 6A, 6B). It was further investigated whether overexpression of these enzymes has effects on LRRK2 endogenous levels (FIGS. 6C, 6D). This suggests that DCP1A and DCP2 may play an important role in AICAR-regulated LRRK2 mRNA decay. It was further asked whether DCP1/2 complex decaps LRRK2 mRNA and causes LRRK2 decay in an in vitro decapping assay. Recombinant protein DCP1A or 2 (FIG. 15B) was incubated with [α-³²P]cap-labeled LRRK2 RNA. The decapping activity was tested by the level of m⁷GDP relative to total RNA (FIG. 6E). DCP1A and DCP2 decapped LRRK2 mRNA as demonstrated by the increased m⁷GDP level (FIG. 6E). These results together suggest that AUF1 interacts with LRRK2 mRNA directly and DCP1/2 is involved in LRRK2 mRNA decay upon AICAr treatment.

### AICAr Effects on LRRK2 Requires AICAr to be Converted to AICAR/ZMP but is Independent of AMPK.

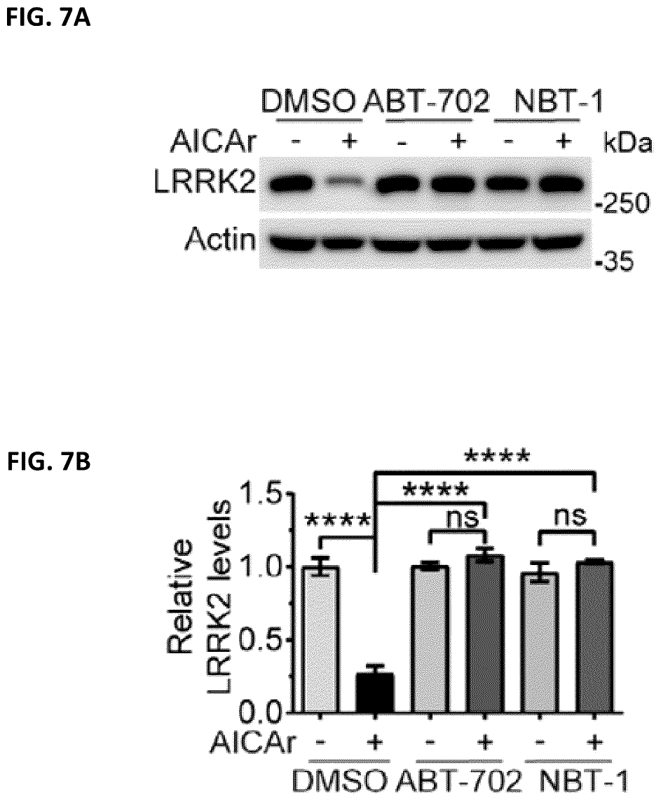

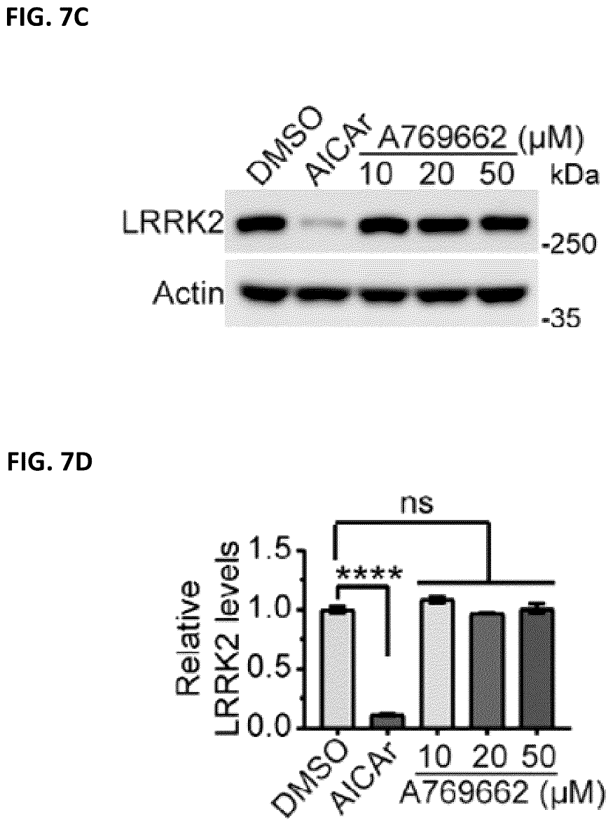

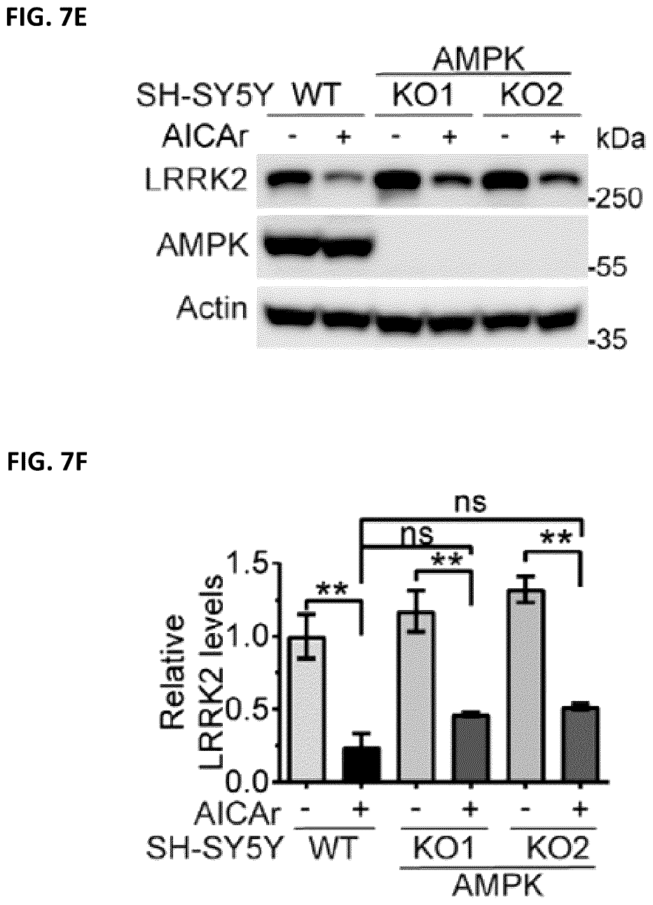

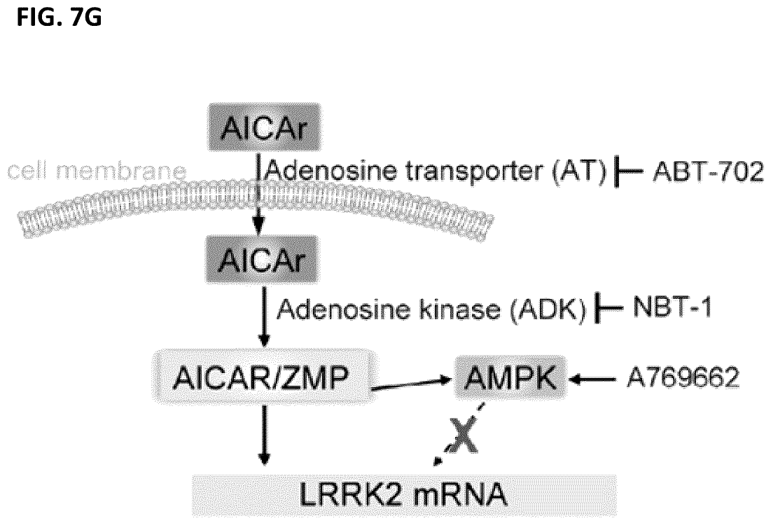

AICAr is a well-known AMP-activated protein kinase (AMPK) activator³⁸. AICAr is transported into cells through adenosine transporter (AT), and then converted into AICAR 5′-monophosphate (ZMP) by adenosine kinase (AK). Subsequently, AICAR/ZMP, an AMP analog, directly activates AMPK^{38,39} (FIG. 7G, model). To determine how AICAr exerts its effect on LRRK2 levels, several types of inhibitors that block the transport or metabolism of AICAr were first employed. NBTI, an AT inhibitor, completely abolished the effect of AICAr on LRRK2 levels (FIGS. 7A, 7B). ABT-702, an AK inhibitor, also completely counteracted the effect of AICAr as evidenced by no changes in LRRK2 levels (FIGS. 7A, 7B). These results clearly show that AICAr must be internalized into cells and converted to AICAR/ZMP to exert its effect on LRRK2. Next, it was asked whether AICAr exerts its effect on LRRK2 through AMPK. To this end, AMPK direct activator A769662 was employed to activate AMPK. However, it was found that A769662 had no obvious effects on LRRK2 levels (FIGS. 7C, 7D). To further confirm this observation, AMPK KO cell line was generated using gRNAs targeting AMPKα1 and AMPKα2 (FIG. 14B) and determined the LRRK2 levels upon AICAr treatment. The effects of AICAr on LRRK2 protein levels are similar in WT and AMPK KO cell line. Together, these data indicate that AICAr exerts its effect on LRRK2 independent of AMPK (FIGS. 7E, 7F, 7G).

### AICAr Rescues LRRK2-Induced Dopaminergic (DA) Neurodegeneration In Vivo in Drosophila.

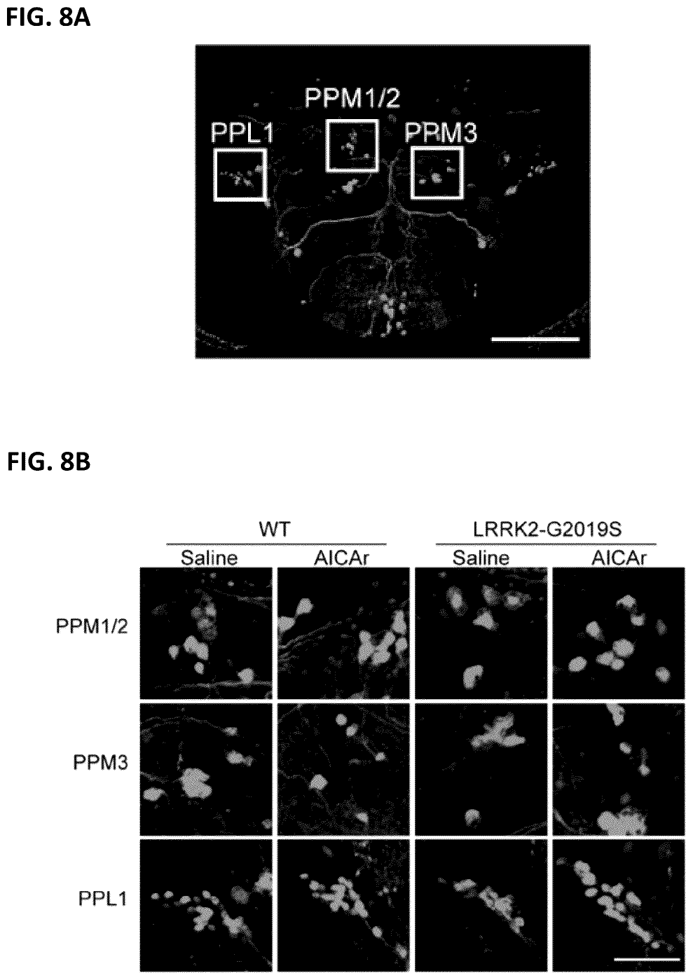

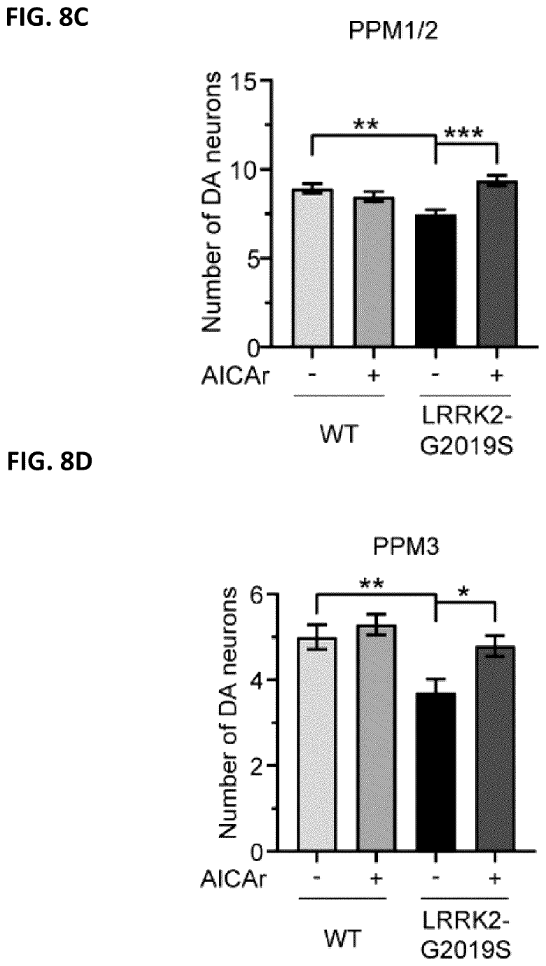

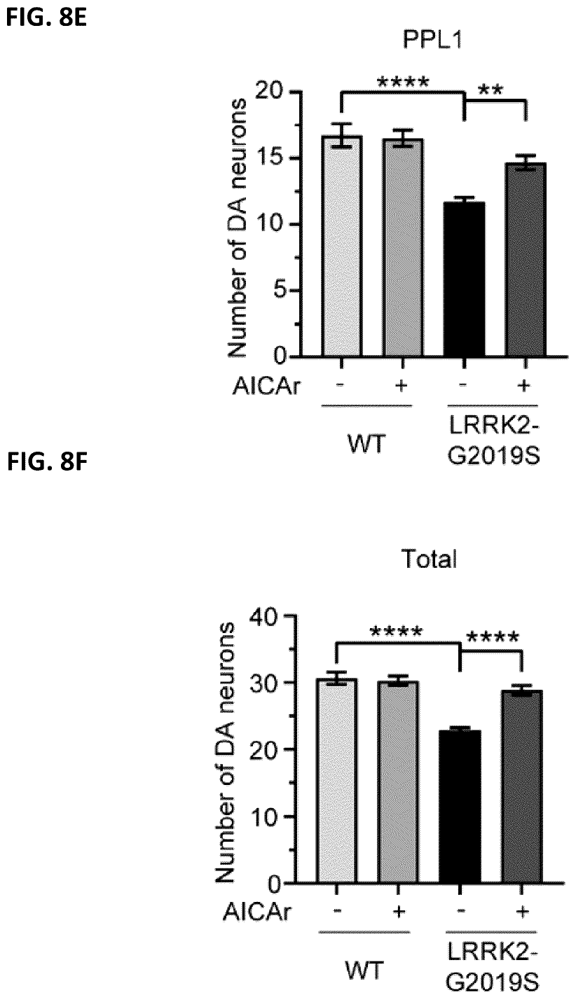

Given the critical role of LRRK2 in DA neurodegeneration, the effects of AICAr on LRRK2-induced DA neurodegeneration were investigated in whole organisms. To this end, LRRK2 Drosophila genetic model in which flies express human LRRK2-G2019S was used. The transgenic flies were crossed with dopa decarboxylase (Ddc)-Gal4>UAS-GFP driver flies to achieve specific expression of the transgene in DA and serotonin neurons. DA neuron number as revealed by GFP fluorescence was monitored in four major DA neuronal clusters in Drosophila adult brain (PPM1/2, PPM3, PPL1) (FIG. 8A and FIG. 8B). Consistent with previous LRRK2 Drosophila models, the present LRRK2 G2019S flies exhibited substantial DA neurodegeneration in the major DA clusters (FIGS. 8C-8E) and total DA neurons (FIG. 8F). Notably, AICAr treatment rescued LRRK2-GS induced DA neurodegeneration as indicated by the increased DA neuron numbers (FIGS. 8C-8F). These results suggest that AICAr suppresses LRRK2-induced DA neurodegeneration.

AICAr Rescues Lipopolysaccharide (LPS)-Induced DA Neurodegeneration and Inflammation in Mice, which Specifically Acts Through LRRK2.

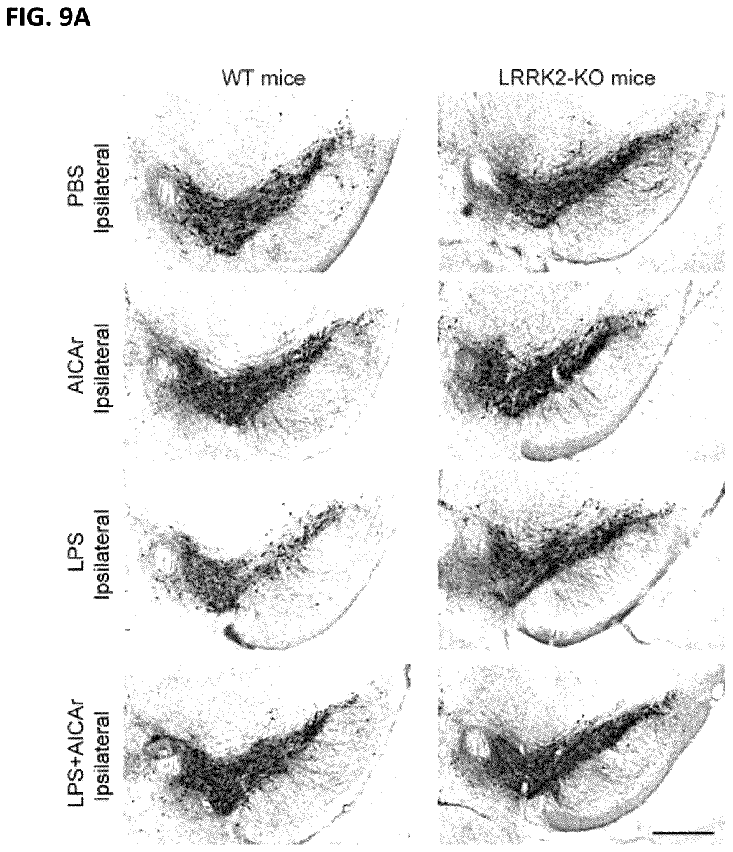

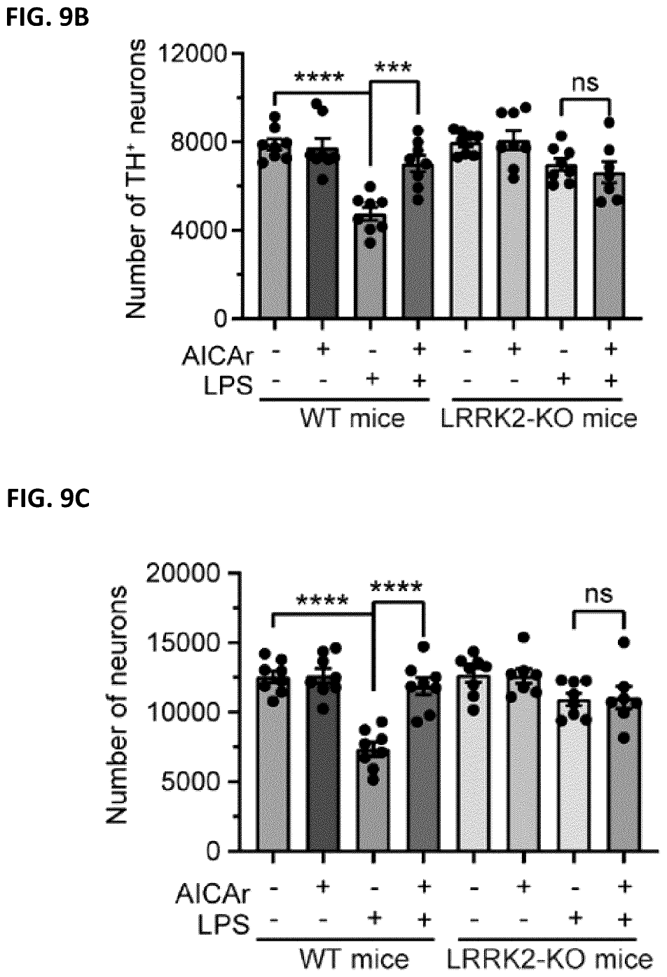

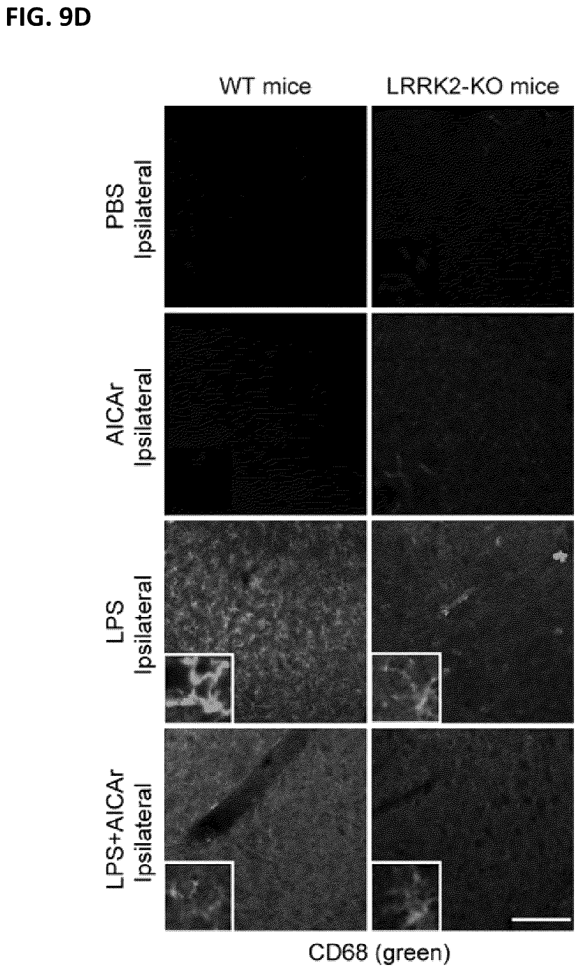

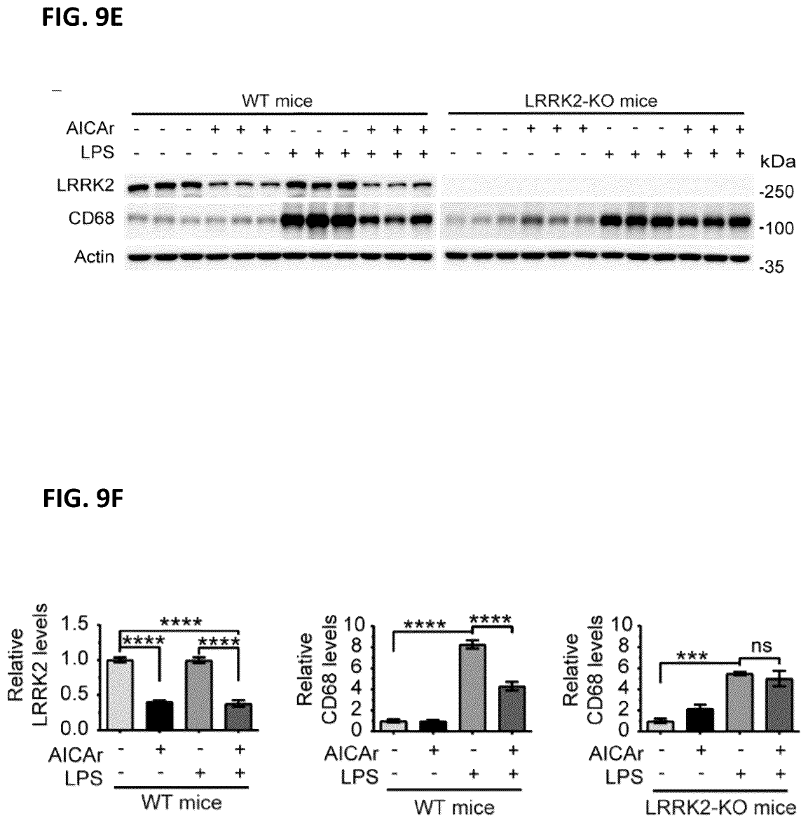

To further study the role of LRRK2 in neurodegeneration and neuroinflammation in mammalian system, the effects of AICAr on neurodegeneration and inflammation was investigated in an LPS mouse model. LPS exposure produces robust and selective DA neurodegeneration in rodents⁴³. LRRK2-deficient is neuroprotection from LPS exposure-induced DA neurodegeneration and inflammation 44. As AICAr markedly suppresses LRRK2 expression levels, it was hypothesized that AICAr suppresses LPS-induced DA neurodegeneration and inflammation, which acts specifically through regulation of LRRK2 levels. To test this hypothesis, LPS was intracranially injected into the substantia nigra pars compacta (SNpc) of WT and LRRK2 KO mice and AICAr was administrated or saline to same stereotaxic coordinates. After 3 weeks of injection, in WT mice with LPS treatment, about 40% TH neuronal loss was observed compared to WT mice injected with PBS (FIGS. 9A, 9B, 9C) and a significant increase of the inflammation marker CD68 levels (FIG. 9D), which is a marker for activated microglia, macrophages and monocytes, was also observed. Strikingly, AICAr injection significantly reduced LPS-induced DA neurodegeneration (FIGS. 9A, 9B, 9C) and CD68 levels (FIG. 9D). To determine whether AICAr acts specifically through LRRK2, LRRK2 KO mice were used. In LRRK2 KO mice, LPS induces about 12% TH neuronal loss which is significantly less than WT mice treated with LPS, suggesting LRRK2 plays an essential role in LPS-induced neurodegeneration. Interestingly, AICAr injection did not significantly rescue LPS-induce neurodegeneration (FIGS. 9A, 9B, 9C) and CD68 levels (FIG. 9D) in LRRK2 KO mice. LPS was further intracranially injected into the striatum, where LRRK2 expression is enriched, in WT and LRRK2 KO mice and AICAr or saline was administrated to the same stereotaxic coordinates. LPS-induced neuroinflammation was examined by Western blot. After 3 weeks of injection, a significant increase of CD68 levels was observed in WT mice with LPS treatment (FIG. 9E and FIG. 9F). Notably, AICAr treatment significantly reduced CD68 levels stimulated by LPS in WT mice but not in LRRK2 KO mice (FIG. 9E and FIG. 9F). A significant decrease of LRRK2 levels was also observed upon AICAr treatment in WT mice (FIG. 9E and FIG. 9F), which is consistent as described before in this study (FIG. 2Q, FIG. 2R). These results suggest AICAr rescues Lipopolysaccharide (LPS)-induced DA neurodegeneration and inflammation in mice, which specifically acts through LRRK2.

### Discussion

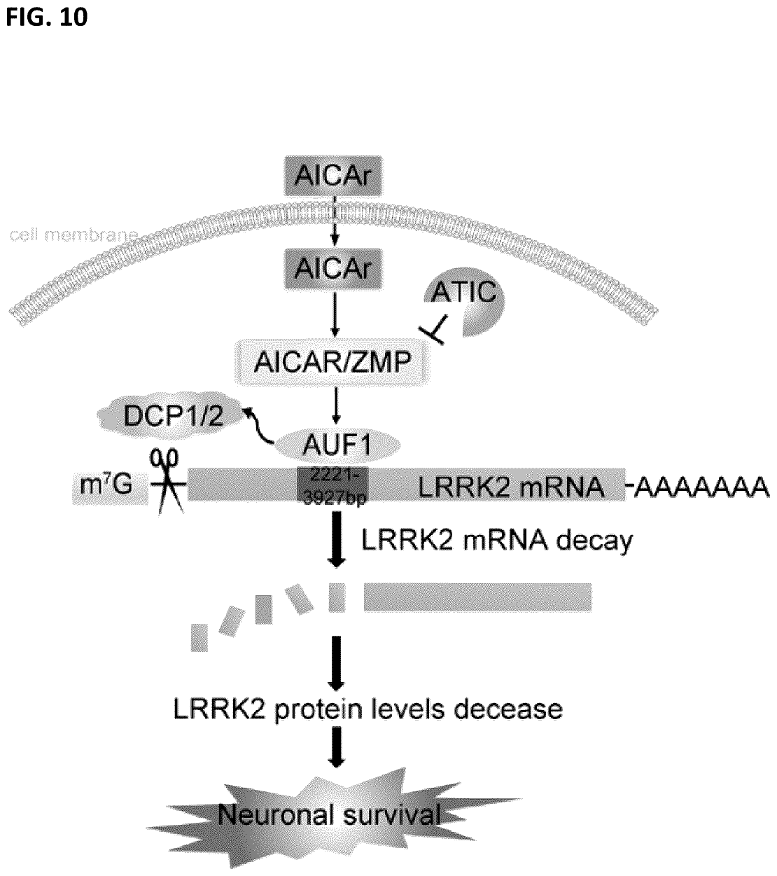

This study, for the first time, determined a novel mechanistic regulation of LRRK2 mRNA level, which acts through an ARE-mediated mRNA decay (AMD). It was discovered that ATIC and AICAr, the precursor of its substrate AICAR/ZMP, dramatically regulate LRRK2 levels and its induced neuronal toxicity. AICAr regulates LRRK2 specifically at mRNA levels through recruiting AUF1 to AU-rich elements (ARE) of LRRK2 mRNA for mRNA decay (FIG. 10 model). Interestingly, the effect of AICAr on LRRK2 requires AICAr to be converted to AICAR/ZMP but is independent of AMPK. The inventors further demonstrated that AICAr administration rescues LRRK2-induced DA neurodegeneration and neuroinflammation in a LRRK2 Drosophila model and a LPS PD model. Thus, this study provides a novel mechanism of ATIC-AICAR-AUF1-LRRK2 axis on LRRK2 regulation, which is implicated in LRRK2-induced DA neurodegeneration and neuroinflammation.

How LRRK2 expression levels are regulated is currently an important yet understudied area in PD pathogenesis. Several studies have focused on the regulation of LRRK2 protein stability. However, the regulation of LRRK2 mRNA decay has not been reported. In eukaryotes, there are two major mRNA decay pathways: nonsense-mediated decay (NMD) and AU-rich element (ARE)-mediated decay (AMD). In AMD, an ARE is comprised of several copies of an AUUUA pentamer or just a U-rich domain only. The presence of AREs is normally in the mRNA 3′-untranslated region (3′-UTR) but is not uncommon to be found in the coding region. Over twenty ARE-binding proteins (AUBPs) have been identified to control AMD. Among those, AUF1 is the most well-known AUBP. Here, the inventors of the present application found that AUF1 binds to a LRRK2 coding region with enriched AREs, while strikingly AUF1 does not bind to a synthesized LRRK2 cDNA with synonymous codons, of which much less AREs are present, and on which AICAr has very minimal effects. More interestingly, when the respective synthesized LRRK2 cDNA were replaced with the AICAr response element identified in native LRRK2 cDNA, the resulting chimeric LRRK2 cDNA response to AICAr treatment reoccurs. This suggests the AREs are critical for LRRK2 mRNA decay. The inventors further analyzed native mouse LRRK2 for AUF1-binding motifs in the AICAr response region (2221-3927 bp) with RBPmap using the same analysis setting as native human LRRK2. The inventors found that native mouse LRRK2 also has several AUF1-binding motifs although less than native human LRRK2. LRRK2 human homolog LRRK1 and other PD genes (GBA, SNCA, PARK7, PRKN, PINK1, VPS35) were also analyzed for AUF1-binding motifs with RBPmap using the same analysis setting as native human LRRK2. Interestingly, none of tested genes contains enriched AUF1-binding motifs in the coding region, except for VPS35 which has a few AUF1-binding motifs and LRRK1 which has only one AUF1-binding motif in the coding region. This explains the phenomenon that no obvious changes of other PD-associated proteins were observed upon AICAr treatment (FIG. 13). Taken together, this strongly suggests that the ATIC-AICAR-AUF1-LRRK2 axis specifically regulates LRRK2 through AMD to mediate PD pathogenesis.

ATIC is the last enzyme in the de novo purine biosynthesis pathway, which plays important roles in various diseases including neurological diseases. Alterations in purine metabolism have strong correlations with PD progression. Particularly, metabolomic profiling in LRRK2 patients revealed significant aberrations of the major molecules in the purine pathway. How purine pathway regulates PD progression or whether the changes in purine pathway are the consequences of PD progression are unknown. The present study provides the first and an attractive mechanistic insight, of which one of the major enzymes ATIC in the purine pathway regulates LRRK2 levels and in turn the neurodegeneration, to connect purine metabolism and PD pathogenesis.

AICAr regulates cellular functions in AMPK-dependent or AMPK-independent manners. Interestingly, both the AMPK direct activator A769662 and the knockout of AMPK in cells do not have significant effects on LRRK2 response to AICAr treatment, indicating that AICAr effects on LRRK2 are independent of AMPK. This could be due to AICAR/ZMP recruiting AUF1 to LRRK2 mRNA or other unidentified AMPK-independent mechanisms, which distinguishes LRRK2 from the other targets of AICAR/ZMP. We also could not exclude the possibility that AUF1 acts as the major RBP for LRRK2 but other RBPs may also exist.

Cell-type or tissue specific mechanisms have been intensively implicated in neurodegenerative diseases. Interestingly, the inventors found significant effects of AICAr on LRRK2 levels in neurons and microglia while no effects in astrocytes, suggesting a cell-specific regulation of LRRK2 levels by AICAr. This indicates that LRRK2 levels may undergo different regulation in astrocytes. Future studies are warranted to dissect the specificity of AICAr-mediated LRRK2 regulation across various cell types/tissues, which may provide new insights to develop AICAr as a specific reagent for regulating LRRK2 level.

While majority of studies have focused on the enzymatic activities of LRRK2 in the field, off-target effects or on-target side effects of LRRK2 enzymatic functions were observed, and increasing studies revealed elevated LRRK2 protein expression in certain PD patient tissues without LRRK2 enzymatic dysfunctions. This study provides the first and a novel regulatory mechanism of LRRK2 function through LRRK2 mRNA decay to reduce LRRK2 levels, which is distinct from the known enzymatic functions of LRRK2.

### Materials and Methods

Animals. LRRK2 GSKI mice were purchased from Taconic (13940)⁴ and LRRK2 KO mice were purchased from JAX Laboratory (16121)⁶. Mice were housed and treated in accordance with the National Institutes of Health (NIH) ‘Guide for the Care and Use of Laboratory Animals’ and Institutional Animal Care and Use Committees of University of Connecticut Health Center. Animals were housed in a 12-hour dark and light cycle with free access to water and food.

Plasmids. The entry clones carrying ATIC, GBA, AUF1, and DCP1A full-length cDNA were obtained from DNASU (clones HsCD00042277, HsCD00515915, HsCD00622694, HsCD00042275). Full-length human ATIC, DCP1A and DCP2 were cloned by PCR into the lentiviral vector pLX-Flag adapted from pLX304 vector (Addgene plasmid, #25890). Full-length human GBA was cloned by Gateway technology into the mammalian expression vector pcDNA3.1-nV5-DEST. MYC-LRRK2-WT was a gift from Dr. Ted Dawson (Addgene plasmid #17609) 7. To generate LRRK2 single mutant, site-directed mutagenesis was carried out using the In-Fusion HD Cloning Plus (Clontech). pENTR221 LRRK2 WT was a gift from Michael J Fox Foundation MJFF (Addgene plasmid, #39529). Full-length human LRRK2, was cloned by Gateway technology into the mammalian expression vector pcDNA3.1-3Flag-DEST, which was generating by replacing a V5 tag with a 3Flag tag from pcDNA3.1-nV5-DEST vector (Invitrogen). Full-length LRRK2 with synonymous codons was cloned into the mammalian expression vector pcDNA3.1 with a 3Flag tag at N-terminus as described previously⁴². pET28a-His6Flag2AUF1(p37), pET28a-His6Flag2AUF1(p40), pET28a-His6Flag2AUF1(p42), and pET28a-His6Flag2AUF1(p45) were gifts from Dr. Yimon Aye (Addgene plasmids, #135939, #135940, #135941, #135942)⁴⁷. Four AUF1 isoforms were cloned by PCR into a lentiviral vector pLX-Flag-GFP adapted from pLX304 vector (Addgene plasmid #25890). Full-length human AUF1 was cloned by Gateway technology into the mammalian expression vector pcDNA3.1-3HA-DEST, which was generating by replacing a V5 tag with a 3HA tag from pcDNA3.1-nV5-DEST vector (Invitrogen).

Antibodies and Reagents. Rabbit monoclonal anti-p-S1292-LRRK2 (ab203181) and rabbit polyclonal anti-GFP (ab290), were obtained from Abcam. Mouse monoclonal anti-LRRK2 (clone N138/6) was from UC Davis/NIH NeuroMab facility. Rabbit monoclonal anti-LRRK2 (D18E12, 13046), rabbit monoclonal anti-AUF1 (D604F, 12382) were obtained from Cell Signalling Technology. Rabbit anti-Tyrosine Hydroxylase polyclonal antibody (NB300-109) and mouse anti-CD68 monoclonal antibody (NB600-985) were obtained from Novus Biologicals. Mouse monoclonal anti-V5 antibody (R96025) was purchased from Thermo Scientific. Mouse monoclonal anti-MYC, anti-V5-HRP, anti-Flag, anti-Flag-HRP, anti-HA, anti-HA-HRP, and anti-ACTIN antibodies were obtained from Sigma-Aldrich. Biotin-SP goat anti-rabbit IgG and, HRP-linked anti-rabbit and anti-mouse IgG antibodies were obtained from Jackson ImmunoResearch Labs. AlexaFluor-488 anti-mouse IgG and AlexaFluor-488 anti-rabbit IgG were obtained from Molecular Probes (Thermo Scientific).

Yeast strains and genetic procedures. Yeast haploid WT strain BY4741 (YSC1048), deletion mutants of yeast ade16 and ade17 and double deletion of ade16 & 17 at genetic background (MATa, his3Δ1, leu20, lys2-0, ura30) were kindly provided by Dr. Bertrand Daignan-Fornier (IBGC, France)⁴⁸. Transformations of yeast were performed using a standard high efficiency lithium acetate procedure⁴⁹. Yeast cells carrying galactose-inducible expression constructs containing LRRK2 fragments¹¹ or empty vector were grown in synthetic complete media lacking uracil (SC-URA) containing glucose (2% dextrose) to repress the GAL) promoter, or in medium containing 2% galactose (to induce the GAL) promoter), to allow induction of expression.

Yeast cell viability assays. WT BY4741 yeast cells and deletion mutants of yeast ATIC homologs carrying galactose-inducible LRRK2 expression constructs were grown overnight at 30° C. in liquid media (SC-URA) containing raffinose to log phase, followed by growth in media containing galactose for a further 6 hrs. Cultures were then normalized for OD 600 nm, 5-fold serially diluted and spotted onto plates containing solid media (SC-URA) with either glucose or galactose as the sole carbon source. Cells were grown at 30° C. for at least 2 days before imaging.

Yeast genome-wide genetic screen. The yeast LRRK2 toxicity modifier screen was performed using haploid-based Synthetic Lethality Analysis (hSLA) modified from previous screenings⁵⁰. Briefly, pYES2-LRRK2-ΔN plasmid was transformed into a deletion haploid collection including about 5000 genes^{31,51}. Haploid deletion mutants carrying LRRK2 plasmid were grown on selectable media containing glucose to suppress LRRK2 expression or on selectable media containing galactose to induce LRRK2 expression. After comparing colony sizes on galactose plates to those on glucose plates, the clones that suppressed or enhanced LRRK2 toxicity were identified. Initial hits from the screen were identified by PCR and sequencing. Each hit was then individually verified by fresh transformations and spotting assays.

Cell culture, transfection, and immunoprecipitation. Human neuroblastoma SH-SY5Y cells and human embryonic kidney (HEK) 293T cells were cultured in DMEM medium supplemented with 10% fetal bovine serum. Transient transfection with MYC-LRRK2-WT, V5-GBA, Flag-DCP1A, or DCP2 was carried out using LipoD293 (Signagen) as per the manufacturer's introductions. After 48 hours, cells were lysed in 2× Laemmli sample buffer (Bio-Rad) and subjected to Western blot, or for immunoprecipitation were washed by PBS (phosphate buffered saline) once, lysed in immunoprecipitation (IP) buffer (1% Triton X-100, 0.5% NP40, 150 mM NaCl, 20 mM HEPES, pH=7.4, 1 mM EGTA, 1 mM EGTA, 1×Complete mini protease inhibitor cocktail (Pierce)) by rotation at 4° C. for 1 hour. Cell lysates were centrifuged at 15,000 rpm for 15 minutes (minutes). Supernatants were incubated with protein-G Dyna beads (Bio-Rad) pre-coated with anti-Flag antibodies following by rotating overnight at 4° C. The Dyna beads were pelleted and stringently washed five times with IP buffer supplemented with 500 mM NaCl. The immunoprecipitated proteins were resuspend in decapping buffer or resolved on SDS/PAGE and subjected to immunoblotting.

Preparations of lentiviruses. The second-generation lentiviral packaging system was employed to produce high-titer lentiviruses. Briefly, HEK 293FT cells were treated with 25 μM chloroquine in Opti-MEM (Gibco) for 2 hours, then pLX-Flag-ATIC, pLX-Flag-DCP1A, pLX-Flag-DCP2, and ATIC-shRNA #6 lentiviral plasmids were transfected into HEK 293FT cells along with viral packaging plasmids (psPAX2 and pMD2.G), and after transfection for 5 hours, cells were treated with 1 μM Na Butyrate. After transfection for 24 and 72 hours, the culture media were refreshed. After transfection for 72 and 96 hours, culture media were collected, centrifuged at 1000 g for 10 minutes and passed through 0.45 μm filters. Viral particles were precipitated by centrifugation at 35,000 g for 2 hours, then resuspended into Opti-MEM and stored at −80° C. as aliquots.

Primary neuronal cultures and viability assay. Primary cortical neuronal cultures were prepared from embryonic day 15-16 CD1 fetal mice (Charles River)¹. Briefly, cortices were dissected and dissociated by Trypsin (Invitrogen). The cells were seeded into 24-well plates pre-coated with poly-L-ornithine (Sigma) and were maintained in Neurobasal medium (Gibco) supplemented with B27 supplement (Gibco) and L-glutamine. The glial cells were inhibited by adding 5-fluoro-20-deoxyuridine (5F2DU, 30 μM, Sigma) at days in vitro (DIV) 4. To assess ATIC in LRRK2-induced toxicity, neurons were first infected with lentiviruses of ATIC-shRNA #6 or control-shRNA at DIV 3, and MYC-LRRK2, DsRed were co-transfected at a plasmid ratio of 10:1 into neurons at DIV 5 using Lipofectamine 2000 reagent (Invitrogen). To assess AICAr in LRRK2-induced toxicity, eGFP plus MYC-LRRK2 WT or G2019S, were co-transfected at a plasmid ratio of 10:1 into neurons at DIV 5 using Lipofectamine 2000 reagent with or without 1 mM AICAr. At 72 hours post-transfection, neurons were fixed with 4% paraformaldehyde (PFA) in PBS. Fixed cells are washed with PBS, permeabilized and blocked for 1 hour with PBS containing 5% goat serum and 0.3% Triton X-100, then detected with rabbit anti-GFP antibody followed by AlexaFluor-488 anti-rabbit IgG antibody. Fluorescent images were collected on a Zeiss Automatic stage microscope with Zen blue software. For each independent experiment, the percent viability of GFP-positive neurons (n>100) was determined and normalized to the control neurons transfected with pEGFP-N1 plus pcDNA3.1 empty vector.

Generation of ATIC, AUF1, and AMPK knockout (KO) SH-SY5Y, and AUF1 KO HEK 293T cell lines by the CRISPR/Cas9 system. The gRNAs targeting the genomic sequence of ATIC and AUF1 (Table 2) were subcloned into plasmid pSpCas9(BB)-2A-Puro(PX459) V2.0 or pSpCas9n(BB)-2A-Puro (PX462) V2.0 (a gift from Dr. Feng Zhang, Addgene, plasmid #62988, #62987) following previous protocol⁵². SH-SY5Y or HEK 293T cells were transfected with specific gRNA plasmids. After 48 hours, cells were selected with 3 ug/ml puromycin (SH-SY5Y cells) or 1.5 ug/ml puromycin (HEK 293T cells) for another 48 hours, and then single cell was plated and maintained in a 96 well plate. Deletion of ATIC, AUF1 and AMPKα1/2 was verified by Western blot with specific antibodies (FIGS. 11A-C). Multiple knockout cell lines were selected.

Generation of AUF1-eGFP stable expression cells. In order to obtain high AUF1 expression in SH-SY5Y cells, AUF(p37)-eGFP, AUF(p40)-eGFP, AUF(p42)-eGFP and AUF(p45)-eGFP were stably expressed in SH-SY5Y cells. Briefly, SH-SY5Y WT or AUF1-KO cells were transfected with pLX-Flag-AUF(p37)-eGFP, pLX-Flag-AUF(p40)-eGFP, pLX-Flag-AUF(p42)-eGFP or pLX-Flag-AUF(p45)-eGFP. After 48 hours, cells were selected with 4 ug/ml blasticidin. Single cell was plated and maintained in a 96 well plate.

Real-Time PCR (qPCR). SH-SY5Y WT or AUF1-KO cells were treated with or without 2 mM AICAr for 24 hours, and total RNA was extracted from 3×10⁵ cells of each treatment using TRIzol™ Reagent (Invitrogen) as per the manufacturer's introductions. Real-Time PCR (qPCR) assay was performed using GoTaq® 2-Step RT-PCR System (Promega) as per the manufacturer's introductions. Briefly, total cDNA was reversely transcribed from RNA pools (1.5 μg RNA in a 10 μl reaction mixture) using random primers. The qPCR reaction was run in CFX96 qPCR detection system (Bio-Rad) with 3 μl cDNA (1:20 dilution) in a 10 μl reaction mixture (Table 3), and set at 95° C. for 2 minutes followed by 40 amplification cycles of 95° C. for 15 seconds, 60° C. for 1 min with a melting curve, 95° C. for 15 seconds, 60° C. for 1 min, a ramp from 60° C. to 95° C. at an 1% rate, and 95° C. for 15 seconds. Melt curves were monitored to assess qPCR amplicons, and critical threshold (Ct) values were collected with an enclosed software. Relative gene expression was analyzed by normalization against Ct values of f-ACTIN (housekeeping gene) and then determined by comparison with expression levels of control sample using the relative expression index (2-ΔΔCt) method.

In vitro RNA transcription. Long RNA probes were transcribed using MEGAscript™ T7 Transcription Kit (Thermo Scientific, AMB13345) and short RNA probes were transcribed using MEGAshortscript™ T7 Transcription Kit (Thermo Scientific, AM1354) as per the manufacturer's introductions. Briefly, LRRK2 fragments were amplified by PCR with a T7 promoter (Table 4). After purification, 200 ng PCR products were added to in vitro RNA transcription reactions. Transcription reactions were incubated at 37° C. overnight. RNA was precipitated and purified by lithium chloride precipitation for long RNA probes or phenol:chloroform extraction and isopropanol precipitation for short RNA probes, aliquoted and stored at −80° C.

RNA-protein pull down assay. RNA-protein pull down assay was performed using Pierce™ Magnetic RNA-Protein Pull-Down Kit (Thermo Scientific, 20164) according to the manufacturer's introductions. Briefly, RNA was first labelled with biotin using Pierce RNA 3′ Desthiobiotinylation Kit (Thermo Scientific, 20163). 100 pmol biotin-labelled RNA was added to streptavidin magnetic beads with gentle agitation at room temperature for 1 hour. After washing, RNA binding magnetic beads were incubated with 200 ug SH-SY5Y cell lysates at 4° C. for 4 hours with gentle agitation. After washing and elution, samples were subjected to SDS-PAGE and coomassie brilliant Blue (CBB) staining. The specific band was cut for mass spectrometric analysis.

Mass spectrometric analysis. Mass spectrometric analysis was performed by the Taplin Biological Mass Spectrometry Facility (Harvard Medical School, MA, USA) and UConn Proteomics & Metabolomics Facility.

RNA immunoprecipitation assay (RIP). SH-SY5Y WT or AUF1-KO cells were treated with or without 1 mM AICAr for 24 hours. Cells were washed by cold PBS once and lysed in radioimmunoprecipitation assay (RIPA) buffer (1% NP40, 0.5% sodium deoxycholate, 0.1% SDS (sodium dodecyl sulfate), 150 mM NaCl, 20 mM HEPES, pH=7.4, 1 mM EGTA, 1× Complete mini protease inhibitor cocktail (Pierce), 1 unit/ul SUPERase.In™ RNase Inhibitor (Invitrogen, AM2694)) by rotation at 4° C. for 1 hour. Cell lysates were centrifuged at 15,000 rpm for 15 minutes. Supernatants were incubated with protein-G Dyna beads (Bio-Rad) pre-coated with anti-AUF1 antibodies following by rotating overnight at 4° C. The Dyna beads were pelleted and washed five times with RIPA buffer. The immunoprecipitated protein and RNA complexes were subjected to RNA extraction using TRIzol™ Reagent (Invitrogen) as per the manufacturer's introductions. Total cDNA was reversely transcribed from immunoprecipitated RNA pools using GoScript™ Reverse Transcriptase with random primers (Promega, A5001) as per the manufacturer's introductions. Binding of LRRK2 RNA to AUF1 was analyzed by PCR using LRRK2 specific primers ((Table 2).

Recombinant protein purification. Recombinant His-AUF1-p37, His-AUF1-p40, His-AUF1-p42, and His-AUF1-p45 were purified in bacteria as previously described⁴⁷. Briefly, pET28a-His6Flag2AUF1(p37), pET28a-His6Flag2AUF1(p40), pET28a-His6Flag2AUF1(p42), and pET28a-His6Flag2AUF1(p45) were transformed into E. coli BL21. Single colony was picked up to start the culture. When cultures reached to optical density (OD) 600 nm=0.5, the expression of His-AUF1-p37, His-AUF1-p40, His-AUF1-p42, and His-AUF1-p45 was induced by 1 mM isopropyl-β-D-thiogalactoside (IPTG) at 37° C. for 4 hours. Cells were collected, washed once with cold PBS, resuspend in lysis buffer (PBS, 5 mM imidazole, 5 mM β-mercaptoethanol (ME), 1× Complete mini protease inhibitor cocktail (Pierce), sonicated for 10 minutes with 8 seconds on/16 seconds off on ice. Lysates were centrifuged at 15000 rpm for 15 minutes and supernatants were incubated with Ni Sepharose (Cytiva, 17531806) overnight at 4° C. with gentle agitation. The sepharose was washed progressively with 20, 50, 100 mM imidazole in wash buffer (PBS, 5 mM β-ME). Recombinant proteins were eluted with elution buffer (PBS, 250 mM imidazole), concentrated, divided into aliquots and stored in 20° C. with 50% glycerol.

RNA electrophoretic mobility shift assay (EMSA). RNA electrophoretic mobility shift assay (EMSA) was performed LightShift™ Chemiluminescent RNA EMSA Kit (Thermo Scientific, 20158) following the manufacturer's instructions. Briefly, short RNA probes were first labelled with biotin using Pierce RNA 3′ Desthiobiotinylation Kit (Thermo Scientific, 20163) following the manufacturer's instructions. After purification of biotin-labelled RNA with phenol:chloroform extraction and isopropanol precipitation, binding reaction was prepared with 2 nM biotin-labelled LRRK2 RNA or control RNA probe and 2 ug recombinant protein at room temperature for 30 minutes, electrophoresed in 6% polyacrylamide gel at 100V for 40 minutes, and then transferred into Nylon membrane (Invitrogen, AM10100) at 400V for 30 minutes on ice. After crosslink at a UV lamp for 5 minutes, the membrane was incubated with stabilized streptavidin-horseradish peroxidase conjugate and imaged on AI600 imager (GE Healthcare).

In vitro decapping assay. In vitro decapping assay was performed as previous described⁵³. Briefly, 62-nt (nucleotides) LRRK2 RNA (Table 4) was first heated at 65° C. to denature secondary structures for 5 minutes and chilled on ice for 5 minutes. The capping reaction was prepared by add 4 ug RNA into a total 40 ul reaction mixture containing (4 μl of 10× capping buffer, 2 ul S-adenosylmethionine (SAM), 2 ul vaccinia capping enzyme (NEB, M2080S), 4 μl (40 ρCi)-[α-³²P] GTP (PerkinElmer), 2 ul SUPERase.In™ RNase Inhibitor (Invitrogen, AM2694)). After incubation at 37° C. for 1 hour. mRNA was purified by phenol:chloroform extraction and isopropanol precipitation. Flag-DCP1A, Flag-DCP2 or pcDNA3.1 vector was transfected into HEK 293T cells (30 ug plasmid per T150 flask for transfection with two T150 flask for each plasmid) using LipoD293 (Signagen) according to manufacturer's introductions. After immunoprecipitation, Flag-DCP1A and Flag-DCP2 were remained in magnetic beads. One tenth of material was used for Western blot. The remaining bead-bound proteins were washed once in decapping buffer (50 mM, Tris-HCl, pH 7.9, 30 mM ammonium sulfate, and 1 mM MgCl2) and resuspended in decapping buffer. The decapping reaction was prepared including beads-bound proteins, 0.1 mM m7G(5′)ppp(5′)G RNA Cap Structure Analog (NEB, S1404S), 0.5 unit/ul RNase inhibitor, 50 ng α-³²P labelled LRRK2 RNA in a total 10 ul volume. The reactions were incubated at 30° C. on a thermomixer at 1500 rpm. After 30 minutes, reactions were stopped by addition of 50 mM EDTA. 5 μl of reaction was loaded on a TLC PEI Cellulose F plate (Millipore, 105725) and separated using 0.75M KH2PO3 (pH=3.4) as a solvent.

Administration of AICAr into LRRK2 Drosophila. Ddc-GAL4 and UAS-GFP fly lines were obtained from Bloomington Stock Center. pUAST-attB-LRRK2 WT and G2019S were microinjected into Drosophila embryos (BestGene Inc.)³⁰. The resulting transgenic flies were crossed with Ddc-GAL4>UAS-GFP, which induces the co-expression of GFP, AP2M1 and LRRK2 in dopamine and serotonin neurons. To study the effects of AICAr on LRRK2-induced dopaminergic neurodegeneration, flies were fed with regular fly food supplemented with 1 mM AICAr immediately at the posteclosion until 9-week-old.

Stereotaxic intracranial injection. Three days before survival surgery, osmotic pumps (ALZET, 1004) were filled with saline, 1 mg/ml or 30 mg/ml AICAr in saline, assembled with brain infusion kits (ALZET, Brain Infusion Kit 2), and incubated in saline at 37° C. 3-month-old mice were anesthetized by isoflurane gas supplemented with 0.5 L/min oxygen. A ˜1.5 cm incision was made midline on the scalp. For mouse striatum injection, one hole was drilled at the following coordinates: anterioposterior=+0.5 mm from bregma; lateral=−2.0 mm from bregma; dorsoventral=−3.5 mm. For mouse substantia nigra pars compacta (SNpc) injection, one hole was drilled at the following coordinates: anterioposterior=−3.2 mm from bregma; lateral=−1.2 mm from bregma; dorsoventral=−4.2 mm. 1 ul LPS (5 ug) or PBS was injected at a rate of 0.2 ul/min using a Hamilton syringe with a 26-gauge blunt needle. After injection, the needle was in place for 5 minutes to minimize backflow. A subcutaneous pocket was made on the animal's back to hold the osmotic pump. The 28-gauge needle from infusion cannulae connected to the osmotic pump with a vinyl catheter tube was inserted into the same hole and then the cannulae was secured to the skull with Loctite 454 (ALZET). The wound was closed with monofilament suture. After one day or 21 days, mice were euthanized and brain tissues around needle were collected and subjected to Western blot or immunohistochemistry.

Immunohistochemistry. At 21st day after injection, mice were anesthetized and perfused with 20 ml ice-cold PBS and then 20 ml ice-cold 4% PFA/PBS. Brains were removed, fixed with 4% PFA at 4° C. overnight, and then immersed in 30% sucrose solution for 24 hours for cryoprotection. Once saturated in sucrose, brains were frozen on dry ice and sliced with a freezing microtome at 40 um. After antigen retrieval with 10 mM sodium citrate (pH 6.0) and 0.05% Tween-20 at 37° C. for 30 minutes, brain sections were washed in PBS for three times, permeabilized with 0.3% Triton X-100 in PBS, blocked with 10% goat serum in PBS, incubated with anti-tyrosine hydroxylase (TH) antibody overnight at 4° C., and then detected with Biotin-SP goat anti-rabbit IgG antibody at room temperature for 2 hours. After washing three time with PBS, the sections were incubated with avidin/biotinylated enzyme complex in PBS (Vector Laboratories, PK-6100) at room temperature for 45 minutes, wash with PBS and stained with 3,3′-Diaminobenzidine (Sigma, D4418). Brain sections with SNpc were counterstained with Niss1 (0.09% Thionin) after TH staining as previously described m, dehydrated in 100% ethanol and cleared in xylene (Sigma) followed by mounting with Cytoseal™ Mountant (Epredia, 83124).

Stereological cell counting. An unbiased stereological method was used to count TH-positive neurons in SNpc in a Zeiss Axiovert 200 microscope equipped with a color camera (MBF bioscience) and stereo investigator software (MBF bioscience). Briefly, counting regions were delineated using a 5× objective according to the mouse brain stereotaxic coordinates s. Counting frame was 40×40 μm with a 200×200 μm grid size. TH positive and Niss1-positive dopaminergic neurons (DA) were counting in a 63× oil-immersion objective at 20 μm thickness. Total cells were counted from 8 serial sections per animal.

Statistical analysis. Statistical analysis was performed with Prism 6.0 software (GraphPad). One-way ANOVA followed by a Tukey's post hoc test was used for data analysis of multiple comparisons and Student's t tests (unpaired, two-tailed) was used for two comparisons as described in the figure legends. Data represent mean±SEM, and p≤0.05 was considered statistically significant. *p<0.05, **p<0.01, ***p<0.001, ****p<0.0001.

TABLE 2
Primers for generating CRISPR/Cas9 sgRNA
to knock out ATIC, AUF1,
AMPK in SH-SY5Y or HEK 293FT cells.

| Primer name | PRIMER SEQUENCE (5′ → 3′) | SEQ ID NO: |
|---|---|---|
| ATIC-sgRNA#1 | GTGCCTGCAGCCATGGCTCC | 1 |
| ATIC-sgRNA#2 | GACCAGATTCAAACCAAGAG | 2 |
| ATIC-sgRNA#3 | GGTTTCTTGCAAATTCCACA | 3 |
| ATIC-sgRNA#4 | AGGGACTGCAAAAGCTCTCA | 4 |
| AUF1-sgRNA#1 | GCGTCTGGAGGCACCGAAGG | 5 |
| AUF1-sgRNA#2 | AATCTTCGCCCCCTCCGACT | 6 |
| AUF1-sgRNA#3 | CGGCTCGGCGGGCGAGCAGG | 7 |
| AUF1-sgRNA#4 | CCCCTGTGTCGCCGCCACCA | 8 |
| AMPKα1- | GCGTGTCACCCAGAATGTAG | 9 |
| sgRNA#1 | | |
| AMPKα1- | ACTCAGTTCCTGGAGAAAGA | 10 |
| sgRNA#2 | | |
| AMPKα1- | GAGGATGCCTGAAAAGCTTG | 11 |
| sgRNA#3 | | |
| AMPKα1- | TGAGGTTCTGAATTTCTCTG | 12 |
| sgRNA#4 | | |
| AMPKα1- | GAATTTTTAAGAACAAGTTG | 13 |
| sgRNA#5 | | |
| AMPKα2- | AGAAGCAGAAGCACGACGGG | 14 |
| sgRNA#1 | | |
| AMPKα2- | CTACGTGCTGGGCGACACGC | 15 |
| sgRNA#2 | | |
| AMPKα2- | TTTGACTACATCTGTAAGCA | 16 |
| sgRNA#3 | | |
| AMPKα2- | GTAATGGAATATGTGTCTGG | 17 |
| sgRNA#4 | | |
| AMPKα2- | GCAGCACCTGAAGTCATCTC | 18 |
| sgRNA#5 | | |
| AMPKα2- | GAATTTCTGAGAACTAGTTG | 19 |
| sgRNA#6 | | |

TABLE 3
Primers for qRT-PCR.

| Gene name | | Primer sequence (5′ → 3′) | SEQ ID NO: | Product size (bp) |
|---|---|---|---|---|
| LRRK2 | Forward | GAGGCGCTTCGAGCTATTT | 20 | 175 |
| | Reverse | CTGAATCCCAGGATTTCCAA | 21 | |
| GBA | Forward | GTCCCAAGCCTTTGAGTAGGG | 22 | 379 |
| | Reverse | GGTGACAGGGCAAGGATGTT | 23 | |
| ACTIN | Forward | CACCAACTGGGACGACAT | 24 | 189 |
| | Reverse | ACAGCCTGGATAGCAACG | 25 | |

TABLE 4
Primers for LRRK2 RNA probes.

| Primer name | | Primer sequence (5′ → 3′) | SEQ ID NO: |
|---|---|---|---|
| T7-LRRK2 probe | Forward | TAATACGACTCACTATAGGGGGATCTTCT | 26 |
| (2221-3927) | | TTAATTTGTCAG | |
| (RNA-protein pull- | Reverse | CCTTATGATGTCTTTGGCTTTAC | 27 |
| down assay) | | | |
| T7-LRRK2 probe | Forward | TAATACGACTCACTATAGGGAATGCAAG | 28 |
| (6490-7581 bp) | | CATTTGGCTGGGC | |
| (RNA-protein pull- | Reverse | CTCAACAGATGTTCGTCTCAT | 29 |
| down assay) | | | |
| T7-Native LRRK2 | Forward | TAATACGACTCACTATAGGGAAATCTAAT | 30 |
| probe (EMSA) | | TCAATTAGTG | |
| | Reverse | GTGAGCAACGCTGTAATACG | 31 |
| T7-Synthesize | Forward | TAATACGACTCACTATAGGGAAGTCTAAC | 32 |
| LRRK2 probe | | TCTATCTCC | |
| (EMSA) | Reverse | GAGAGCAACGCTGGAGGAC | 33 |

### REFERENCES

1 Cookson, M. R. The role of leucine-rich repeat kinase 2 (LRRK2) in Parkinson's disease. Nat Rev Neurosci 11, 791-797, doi:nrn2935 [pii]10.1038/nrn2935 (2010).
2 Mata, I. F., Wedemeyer, W. J., Farrer, M. J., Taylor, J. P. & Gallo, K. A. LRRK2 in Parkinson's disease: protein domains and functional insights. Trends Neurosci 29, 286-293, doi:S0166-2236(06)00069-5 [pii]10.1016/j.tins.2006.03.006 (2006).
3 Pirooznia, S. K., Rosenthal, L. S., Dawson, V. L. & Dawson, T. M. Parkinson Disease: Translating Insights from Molecular Mechanisms to Neuroprotection. Pharmacol Rev 73, 33-97, doi:10.1124/pharmrev.120.000189 (2021).
4 Lees, A. J., Hardy, J. & Revesz, T. Parkinson's disease. Lancet 373, 2055-2066, doi:10.1016/S0140-6736(09)60492-X (2009).
5 Cookson, M. R. LRRK2 Pathways Leading to Neurodegeneration. Curr Neurol Neurosci Rep 15, 42, doi:10.1007/si1910-015-0564-y (2015).
6 Smith, W. W. et al. Kinase activity of mutant LRRK2 mediates neuronal toxicity. Nat Neurosci 9, 1231-1233, doi:nn1776 [pii]10.1038/nn1776 (2006).
7 West, A. B. et al. Parkinson's disease-associated mutations in leucine-rich repeat kinase 2 augment kinase activity. Proc Nat Acad Sci USA 102, 16842-16847, doi:0507360102 [pii]10.1073/pnas.0507360102 (2005).
8 Greggio, E. et al. Kinase activity is required for the toxic effects of mutant LRRK2/dardarin. Neurobiol Dis 23, 329-341, doi:S0969-9961(06)00071-4 [pii]10.1016/j.nbd.2006.04.001 (2006).
9 Steger, M. et al. Phosphoproteomics reveals that Parkinson's disease kinase LRRK2 regulates a subset of Rab GTPases. Elife 5, doi:10.7554/eLife.12813 (2016).
10 Martin, I. et al. Ribosomal protein s15 phosphorylation mediates LRRK2 neurodegeneration in Parkinson's disease. Cell 157, 472-485, doi:S0092-8674(14)00212-8 [pii]10.1016/j.cell.2014.01.064 (2014).
11 Xiong, Y. et al. GTPase activity plays a key role in the pathobiology of LRRK2. PLoS Genet 6, e1000902, doi:10.1371/journal.pgen.1000902 (2010).
12 Cookson, M. R. Mechanisms of Mutant LRRK2 Neurodegeneration. Adv Neurobiol 14, 227-239, doi:10.1007/978-3-319-49969-7_12 (2017).
13 Rudenko, I. N. et al. The G2385R variant of leucine-rich repeat kinase 2 associated with Parkinson's disease is a partial loss-of-function mutation. Biochem J 446, 99-111, doi:10.1042/BJ20120637 (2012).
14 West, A. B. et al. Parkinson's disease-associated mutations in LRRK2 link enhanced GTP-binding and kinase activities to neuronal toxicity. Hum Mol Genet 16, 223-232, doi:dd1471 [pii]10.1093/hmg/dd1471 (2007).
15 Nalls, M. A. et al. Identification of novel risk loci, causal insights, and heritable risk for Parkinson's disease: a meta-analysis of genome-wide association studies. Lancet Neurol 18, 1091-1102, doi:10.1016/S1474-4422(19)30320-5 (2019).
16 Trabzuni, D. et al. Fine-mapping, gene expression and splicing analysis of the disease associated LRRK2 locus. PLoS One 8, e70724, doi:10.1371/journal.pone.0070724 (2013).
17 Heckman, M. G. et al. Fine-mapping of the non-coding variation driving the Caucasian LRRK2 GWAS signal in Parkinson's disease. Parkinsonism Relat Disord 83, 22-30, doi:10.1016/j.parkreldis.2020.12.016 (2021).
18 Lake, J. et al. Coding and Noncoding Variation in LRRK2 and Parkinson's Disease Risk. Mov Disord, doi:10.1002/mds.28787 (2021).
19 Dzamko, N. et al. LRRK2 levels and phosphorylation in Parkinson's disease brain and cases with restricted Lewy bodies. Mov Disord 32, 423-432, doi:10.1002/mds.26892 (2017).
20 Cho, H. J. et al. MicroRNA-205 regulates the expression of Parkinson's disease-related leucine-rich repeat kinase 2 protein. Hum Mol Genet 22, 608-620, doi:10.1093/hmg/dds470 (2013).
21 Guerreiro, P. S. et al. LRRK2 interactions with alpha-synuclein in Parkinson's disease brains and in cell models. J Mol Med (Berl) 91, 513-522, doi:10.1007/s00109-012-0984-y (2013).
22 Skibinski, G., Nakamura, K., Cookson, M. R. & Finkbeiner, S. Mutant LRRK2 toxicity in neurons depends on LRRK2 levels and synuclein but not kinase activity or inclusion bodies. J Neurosci 34, 418-433, doi:10.1523/JNEUROSCI.2712-13.2014 (2014).
23 Zhao, H. T. et al. LRRK2 Antisense Oligonucleotides Ameliorate alpha-Synuclein Inclusion Formation in a Parkinson's Disease Mouse Model. Mol Ther Nucleic Acids 8, 508-519, doi:10.1016/j.omtn.2017.08.002 (2017).
24 Korecka, J. A. et al. Splice-Switching Antisense Oligonucleotides Reduce LRRK2 Kinase Activity in Human LRRK2 Transgenic Mice. Mol Ther Nucleic Acids 21, 623-635, doi:10.1016/j.omtn.2020.06.027 (2020).
25 Ko, H. S. et al. CHIP regulates leucine-rich repeat kinase-2 ubiquitination, degradation, and toxicity. Proc Natl Acad Sci USA 106, 2897-2902, doi:10.1073/pnas.0810123106 (2009).
26 Kargbo, R. B. Degradation of LRRK2 in the Treatment of Parkinson's Disease. ACS Med Chem Lett 11, 2070-2071, doi:10.1021/acsmedchemlett.0c00453 (2020).
27 Herzig, M. C. et al. LRRK2 protein levels are determined by kinase function and are crucial for kidney and lung homeostasis in mice. Hum Mol Genet 20, 4209-4223, doi:10.1093/hmg/ddr348 (2011).
28 Biosa, A. et al. GTPase activity regulates kinase activity and cellular phenotypes of Parkinson's disease-associated LRRK2. Hum Mol Genet 22, 1140-1156, doi:10.1093/hmg/dds522 (2013).
29 Lobbestael, E. et al. Pharmacological LRRK2 kinase inhibition induces LRRK2 protein destabilization and proteasomal degradation. Sci Rep 6, 33897, doi:10.1038/srep33897 (2016).
30 Liu, Q., Bautista-Gomez, J., Higgins, D. A., Yu, J. & Xiong, Y. Dysregulation of the AP2M1 phosphorylation cycle by LRRK2 impairs endocytosis and leads to dopaminergic neurodegeneration. Sci Signal 14, doi:10.1126/scisignal.abg3555 (2021).
31 Pan, X. et al. dSLAM analysis of genome-wide genetic interactions in Saccharomyces cerevisiae. Methods 41, 206-221, doi:10.1016/j.ymeth.2006.07.033 (2007).
32 Jorgensen, N. D. et al. The WD40 domain is required for LRRK2 neurotoxicity. PLoS One 4, e8463, doi:10.1371/joumal.pone.0008463 (2009).
33 Smith, W. W. et al. Leucine-rich repeat kinase 2 (LRRK2) interacts with parkin, and mutant LRRK2 induces neuronal degeneration. Proc Natl Acad Sci USA 102, 18676-18681, doi:0508052102 [pii]10.1073/pnas.0508052102 (2005).
34 Gratacos, F. M. & Brewer, G. The role of AUF1 in regulated mRNA decay. Wiley Interdiscip Rev RNA 1, 457-473, doi:10.1002/wrna.26 (2010).
35 Paz, I., Kosti, I., Ares, M., Jr., Cline, M. & Mandel-Gutfreund, Y. RBPmap: a web server for mapping binding sites of RNA-binding proteins. Nucleic Acids Res 42, W361-367, doi:10.1093/nar/gku406 (2014).
36 Clark, A., Dean, J., Tudor, C. & Saklatvala, J. Post-transcriptional gene regulation by MAP kinases via AU-rich elements. Front Biosci (Landmark Ed) 14, 847-871, doi:10.2741/3282 (2009).
37 Ling, S. H., Qamra, R. & Song, H. Structural and functional insights into eukaryotic mRNA decapping. Wiley Interdiscip Rev RNA 2, 193-208, doi:10.1002/wrna.44 (2011).
38 Corton, J. M., Gillespie, J. G., Hawley, S. A. & Hardie, D. G. 5-aminoimidazole-4-carboxamide ribonucleoside. A specific method for activating AMP-activated protein kinase in intact cells? Eur J Biochem 229, 558-565, doi:10.1111/j.1432-1033.1995.tb20498.x (1995).
39 Hardie, D. G. Minireview: the AMP-activated protein kinase cascade: the key sensor of cellular energy status. Endocrinology 144, 5179-5183, doi:10.1210/en.2003-0982 (2003).
40 Friggi-Grelin, F. et al. Targeted gene expression in Drosophila dopaminergic cells using regulatory sequences from tyrosine hydroxylase. J Neurobiol 54, 618-627, doi:10.1002/neu.10185 (2003).
41 Liu, Z. et al. A Drosophila model for LRRK2-linked parkinsonism. Proc Natl Acad Sci USA 105, 2693-2698, doi:0708452105 [pii]10.1073/pnas.0708452105 (2008).
42 Xiong, Y., Yuan, C., Chen, R., Dawson, T. M. & Dawson, V. L. ArfGAP1 is a GTPase activating protein for LRRK2: reciprocal regulation of ArfGAP1 by LRRK2. J Neurosci 32, 3877-3886, doi:32/11/3877 [pii]10.1523/JNEUROSCI.4566-11.2012 (2012).
43 Liu, M. & Bing, G. Lipopolysaccharide animal models for Parkinson's disease. Parkinsons Dis 2011, 327089, doi:10.4061/2011/327089 (2011).
44 Daher, J. P., Volpicelli-Daley, L. A., Blackburn, J. P., Moehle, M. S. & West, A. B. Abrogation of alpha-synuclein-mediated dopaminergic neurodegeneration in LRRK2-deficient rats. Proc Natl Acad Sci USA 111, 9289-9294, doi:10.1073/pnas.1403215111 (2014).
45 Matikainen-Ankney, B. A. et al. Altered Development of Synapse Structure and Function in Striatum Caused by Parkinson's Disease-Linked LRRK2-G2019S Mutation. J Neurosci 36, 7128-7141, doi:10.1523/JNEUROSCI.3314-15.2016 (2016).
46 Tong, Y. et al. Loss of leucine-rich repeat kinase 2 causes impairment of protein degradation pathways, accumulation of alpha-synuclein, and apoptotic cell death in aged mice. Proc Natl Acad Sci USA 107, 9879-9884, doi:10.1073/pnas.1004676107 (2010).
47 Poganik, J. R. et al. Post-transcriptional regulation of Nrf2-mRNA by the mRNA-binding proteins HuR and AUF1. FASEB J 33, 14636-14652, doi:10.1096/fj.201901930R (2019).
48 Rebora, K., Desmoucelles, C., Borne, F., Pinson, B. & Daignan-Fornier, B. Yeast AMP pathway genes respond to adenine through regulated synthesis of a metabolic intermediate. Mol Cell Biol 21, 7901-7912, doi:10.1128/MCB.21.23.7901-7912.2001 (2001).
49 Gietz, R. D. & Woods, R. A. Transformation of yeast by lithium acetate/single-stranded carrier DNA/polyethylene glycol method. Methods Enzymol 350, 87-96, doi:10.1016/s0076-6879(02)50957-5 (2002).
50 Meluh, P. B. et al. Analysis of genetic interactions on a genome-wide scale in budding yeast: diploid-based synthetic lethality analysis by microarray. Methods Mol Biol 416, 221-247, doi:10.1007/978-1-59745-321-9_15 (2008).
51 Pan, X. et al. A robust toolkit for functional profiling of the yeast genome. Mol Cell 16, 487-496, doi:10.1016/j.molcel.2004.09.035 (2004).
52 Ran, F. A. et al. Genome engineering using the CRISPR-Cas9 system. Nat Protoc 8, 2281-2308, doi:10.1038/nprot.2013.143 (2013).
53 Rzeczkowski, K. et al. c-Jun N-terminal kinase phosphorylates DCPla to control formation of P bodies. J Cell Biol 194, 581-596, doi:10.1083/jcb.201006089 (2011).
54 Xiong, Y. et al. Robust kinase- and age-dependent dopaminergic and norepinephrine neurodegeneration in LRRK2 G2019S transgenic mice. Proc Natl Acad Sci USA 115, 1635-1640, doi:10.1073/pnas.1712648115 (2018).
55 Paxinos, G. & Franklin, K. B. J. The mouse brain in stereotaxic coordinates. Compact 2nd edn, (Elsevier Academic Press, 2004).

### EQUIVALENTS

Those skilled in the art will recognize, or be able to ascertain using no more than routine experimentation, many equivalents to the specific embodiments and methods described herein. Such equivalents are intended to be encompassed by the scope of the following claims.

1. A method of treating a Leucine rich repeat kinase 2 (LRRK2)-associated disorder or condition in a subject in need thereof, comprising administering to the subject an effective amount of an agent that decreases the expression of LRRK2, wherein the agent induces LRRK2 mRNA decay, thereby treating the LRRK2-associated disorder or condition in the subject.

2. A method of reducing or preventing neuronal cell death and/or reducing neurodegeneration and/or neuroinflammation in a subject in need thereof, comprising administering to the subject an effective amount of an agent that decreases the expression of LRRK2, wherein the agent induces LRRK2 mRNA decay, thereby reducing or preventing neuronal cell death and/or reducing neurodegeneration and/or neuroinflammation in the subject.

3. The method of claim 2, wherein the neuronal death is LRRK2-mediated neuronal cell death.

4. (canceled)

5. (canceled)

6. The method of claim 1, wherein the LRRK2-associated disorder or condition is a neurodegenerative disease, an inflammatory disease, or cancer.

7. The method of claim 6,
(a) wherein the neurodegenerative disease is Parkinson's disease or Alzheimer's disease;
(b) wherein the inflammatory disease is selected from the group consisting of Crohn's disease, inflammatory bowel disease, ulcerative colitis, leprosy, amyotrophic lateral sclerosis, rheumatoid arthritis, and ankylosing spondylitis; and/or
(c) wherein the cancer is selected from the group consisting of kidney cancer, breast cancer, prostate cancer, ovarian cancer, cervical cancer, skin cancer, pancreatic cancer, colorectal cancer, renal cancer, liver cancer, brain cancer, lung cancer, lymphoma, leukemia, multiple myeloma, and any combination thereof.

8-11. (canceled)

12. The method of claim 1, wherein the subject is a human subject.

13. The method of claim 1,
(a) wherein the agent is an inhibitor of 5-aminoimidazole-4-carboxamide ribonucleotide formyltransferase (ATIC);
optionally, wherein the ATIC inhibitor is selected from the group consisting of a small molecule, an antagonist antibody of ATIC, or antigen-binding fragment thereof, an antisense agent targeting ATIC, a double stranded RNA agent targeting ATIC, an RNA-guided nuclease targeting ATIC, an ATIC fusion protein, and an ATIC inhibitory peptide;
(b) wherein the agent is 5-aminoimidazole-4-carboxamide ribonucleotide (AICAR/ZMP) or 5-aminoimidazole-4-carboxamide riboside (AICAr);
(c) wherein the agent increases the expression and/or activity of AU-rich element RNA binding protein 1 (AUF);
optionally wherein the agent is selected from the group consisting of a small molecule activator of AUF1, an agonist antibody of AUF1, or antigen-binding fragment thereof, an AUF1 protein, a nucleic acid encoding the AUF1 protein, or a protein and a nucleic acid that activates the transcription and/or translation of AUF1;
(d) wherein the agent increases the expression and/or activity of mRNA decapping enzyme 1 (DCP1) and/or mRNA decapping enzyme 2 (DCP2);
Optionally, wherein the agent is selected from the group consisting of a small molecule activator of DCP1 and/or DCP2, an agonist antibody of DCP1 and/or DCP2, or antigen-binding fragment thereof, a DCP1 and/or DCP2 protein, a nucleic acid encoding the DCP1 and/or DCP2 protein, or a protein and a nucleic acid that activates the transcription and/or translation of DCP1 and/or DCP2; and/or
(e) wherein the agent does not modulate the enzymatic activity of LRRK2.

14-20. (canceled)

21. The method of claim 1, further comprising administering to the subject an additional therapeutic agent;
optionally, wherein the additional therapeutic agent comprises levodopa, carbidopa, a dopamine agonist, a monoamine oxidase B (MAO B) inhibitor, a catechol O-methyltransferase (COMT) inhibitor, an anticholinergic, or an adenosine receptor antagonist.

22. (canceled)

23. A method of reducing LRRK2 expression in a cell, comprising contacting the cell with an agent that induces LRRK2 mRNA decay, thereby reducing LRRK2 expression in the cell.

24. The method of claim 23, wherein the cell is a neuron, a microglia, or a fibroblast.

25. The method of claim 23 wherein the contacting occurs in vitro.

26. The method of claim 23 wherein the cell is within a subject.

27. The method of claim 26, wherein the subject is a human subject; and/or wherein the subject has an LRRK2-associated disorder or condition.

28. (canceled)

29. The method of claim 27, wherein the LRRK2-associated disorder or condition is a neurodegenerative disease, an inflammatory disease, or cancer.

30. The method of claim 29,
(a) wherein the neurodegenerative disease is Parkinson's disease or Alzheimer's disease;
(b) wherein the inflammatory disease is selected from the group consisting of Crohn's disease, inflammatory bowel disease, ulcerative colitis, leprosy, amyotrophic lateral sclerosis, rheumatoid arthritis, and ankylosing spondylitis; and/or
(c) wherein the cancer is selected from the group consisting of kidney cancer, breast cancer, prostate cancer, ovarian cancer, cervical cancer, skin cancer, pancreatic cancer, colorectal cancer, renal cancer, liver cancer, brain cancer, lung cancer, lymphoma, leukemia, multiple myeloma, and any combination thereof.

31-34. (canceled)

35. The method of claim 23,
(a) wherein the agent is an inhibitor of 5-aminoimidazole-4-carboxamide ribonucleotide formyltransferase (ATIC);
optionally, wherein the ATIC inhibitor is selected from the group consisting of a small molecule, an antagonist antibody of ATIC, or antigen-binding fragment thereof, an antisense agent targeting ATIC, a double stranded RNA agent targeting ATIC, an RNA-guided nuclease targeting ATIC, an ATIC fusion protein, and an ATIC inhibitory peptide;
(b) wherein the agent is 5-aminoimidazole-4-carboxamide ribonucleotide (AICAR/ZMP) or 5-aminoimidazole-4-carboxamide riboside (AICAr);
(c) wherein the agent increases the expression and/or activity of AU-rich element RNA binding protein 1 (AUF);
optionally, wherein the agent is selected from the group consisting of a small molecule activator of AUF1, an agonist antibody of AUF1, or antigen-binding fragment thereof, an AUF1 protein, a nucleic acid encoding the AUF1 protein, or a protein and a nucleic acid that activates the transcription and/or translation of AUF1;
(d) wherein the agent increases the expression and/or activity of mRNA decapping enzyme 1 (DCP1) and/or mRNA decapping enzyme 2 (DCP2);
optionally, wherein the agent is selected from the group consisting of a small molecule activator of DCP1 and/or DCP2, an agonist antibody of DCP1 and/or DCP2, or antigen-binding fragment thereof, a DCP1 and/or DCP2 protein, a nucleic acid encoding the DCP1 and/or DCP2 protein, or a protein and a nucleic acid that activates the transcription and/or translation of DCP1 and/or DCP2; and/or
(e) wherein the agent does not modulate the enzymatic activity of LRRK2.

36-44. (canceled)


SEQUENCE LISTING (33 sequences)

SEQ ID NO: 1 - DNA, 20 bp, synthetic construct
  source 1..20: mol_type = other DNA
gtgcctgcagccatggctcc

SEQ ID NO: 2 - DNA, 20 bp, synthetic construct
  source 1..20: mol_type = other DNA
gaccagattcaaaccaagag

SEQ ID NO: 3 - DNA, 20 bp, synthetic construct
  source 1..20: mol_type = other DNA
ggtttcttgcaaattccaca

SEQ ID NO: 4 - DNA, 20 bp, synthetic construct
  source 1..20: mol_type = other DNA
agggactgcaaaagctctca

SEQ ID NO: 5 - DNA, 20 bp, synthetic construct
  source 1..20: mol_type = other DNA
gcgtctggaggcaccgaagg

SEQ ID NO: 6 - DNA, 20 bp, synthetic construct
  source 1..20: mol_type = other DNA
aatcttcgccccctccgact

SEQ ID NO: 7 - DNA, 20 bp, synthetic construct
  source 1..20: mol_type = other DNA
cggctcggcgggcgagcagg

SEQ ID NO: 8 - DNA, 20 bp, synthetic construct
  source 1..20: mol_type = other DNA
cccctgtgtcgccgccacca

SEQ ID NO: 9 - DNA, 20 bp, synthetic construct
  source 1..20: mol_type = other DNA
gcgtgtcacccagaatgtag

SEQ ID NO: 10 - DNA, 20 bp, synthetic construct
  source 1..20: mol_type = other DNA
actcagttcctggagaaaga

SEQ ID NO: 11 - DNA, 20 bp, synthetic construct
  source 1..20: mol_type = other DNA
gaggatgcctgaaaagcttg

SEQ ID NO: 12 - DNA, 20 bp, synthetic construct
  source 1..20: mol_type = other DNA
tgaggttctgaatttctctg

SEQ ID NO: 13 - DNA, 20 bp, synthetic construct
  source 1..20: mol_type = other DNA
gaatttttaagaacaagttg

SEQ ID NO: 14 - DNA, 20 bp, synthetic construct
  source 1..20: mol_type = other DNA
agaagcagaagcacgacggg

SEQ ID NO: 15 - DNA, 20 bp, synthetic construct
  source 1..20: mol_type = other DNA
ctacgtgctgggcgacacgc

SEQ ID NO: 16 - DNA, 20 bp, synthetic construct
  source 1..20: mol_type = other DNA
tttgactacatctgtaagca

SEQ ID NO: 17 - DNA, 20 bp, synthetic construct
  source 1..20: mol_type = other DNA
gtaatggaatatgtgtctgg

SEQ ID NO: 18 - DNA, 20 bp, synthetic construct
  source 1..20: mol_type = other DNA
gcagcacctgaagtcatctc

SEQ ID NO: 19 - DNA, 20 bp, synthetic construct
  source 1..20: mol_type = other DNA
gaatttctgagaactagttg

SEQ ID NO: 20 - DNA, 19 bp, synthetic construct
  source 1..19: mol_type = other DNA
gaggcgcttcgagctattt

SEQ ID NO: 21 - DNA, 20 bp, synthetic construct
  source 1..20: mol_type = other DNA
ctgaatcccaggatttccaa

SEQ ID NO: 22 - DNA, 21 bp, synthetic construct
  source 1..21: mol_type = other DNA
gtcccaagcctttgagtaggg

SEQ ID NO: 23 - DNA, 20 bp, synthetic construct
  source 1..20: mol_type = other DNA
ggtgacagggcaaggatgtt

SEQ ID NO: 24 - DNA, 18 bp, synthetic construct
  source 1..18: mol_type = other DNA
caccaactgggacgacat

SEQ ID NO: 25 - DNA, 18 bp, synthetic construct
  source 1..18: mol_type = other DNA
acagcctggatagcaacg

SEQ ID NO: 26 - DNA, 41 bp, synthetic construct
  source 1..41: mol_type = other DNA
taatacgactcactatagggggatcttctttaatttgtcag

SEQ ID NO: 27 - DNA, 23 bp, synthetic construct
  source 1..23: mol_type = other DNA
ccttatgatgtctttggctttac

SEQ ID NO: 28 - DNA, 41 bp, synthetic construct
  source 1..41: mol_type = other DNA
taatacgactcactatagggaatgcaagcatttggctgggc

SEQ ID NO: 29 - DNA, 21 bp, synthetic construct
  source 1..21: mol_type = other DNA
ctcaacagatgttcgtctcat

SEQ ID NO: 30 - DNA, 39 bp, synthetic construct
  source 1..39: mol_type = other DNA
taatacgactcactatagggaaatctaattcaattagtg

SEQ ID NO: 31 - DNA, 20 bp, synthetic construct
  source 1..20: mol_type = other DNA
gtgagcaacgctgtaatacg

SEQ ID NO: 32 - DNA, 38 bp, synthetic construct
  source 1..38: mol_type = other DNA
taatacgactcactatagggaagtctaactctatctcc

SEQ ID NO: 33 - DNA, 19 bp, synthetic construct
  source 1..19: mol_type = other DNA
gagagcaacgctggaggac